(19) United States
(12) Patent Application Publication
(10) Pub. No.: US 2024/0382586 A1
(43) Pub. Date: Nov. 21, 2024

(54) ANTIBODIES AGAINST SARS-COV-2 AND USES THEREOF IN THE MEDICAL FIELD

(71) Applicant: TAKIS S.R.L., Rome (IT)

(72) Inventors: Giuseppe ROSCILLI, Rome (IT); Luigi AURISICCHIO, Rome (IT); Emanuele MARRA, Rome (IT); Alessia MUZI, Rome (IT); Emanuela D'ACUNTO, Rome (IT)

(21) Appl. No.: 18/692,600

(22) PCT Filed: Sep. 15, 2022
(86) PCT No.: PCT/IT2022/050250

§ 371 Date: Mar. 15, 2024

(30) Foreign Application Priority Data
Sep. 16, 2021 (IT) 102021000023816

(51) Int. Cl. A61K 39/42; A61K 39/00; A61P 31/14; C07K 16/10; G01N 33/569

(52) U.S. Cl. CPC A61K 39/42; A61P 31/14; C07K 16/1003; G01N 33/56983; A61K 2039/507; C07K 2317/24; C07K 2317/565; C07K 2317/76; G01N 2333/165

(57) ABSTRACT

The present invention relates to antibodies against SARS-CoV-2 and uses thereof in the medical field for the prevention and treatment of SARS-CoV-2 infection and COVID-19 disease.

The present invention relates to antibodies against SARS-CoV-2 and uses thereof in the medical field.

In particular, the invention relates to antibodies against SARS-CoV-2 and uses thereof in the medical field for the prevention and treatment of SARS-CoV-2 infection and COVID-19 disease. It is well known that the coronavirus disease of 2019 (COVID-19) was declared a pandemic in March 2020 by the World Health Organization (1), with acute respiratory clinical manifestations and symptoms, pathological inflammation, and multi-organ dysfunctions. In just a few months, from December 2019, COVID-19 spread across the world with over 151,803,822 cases and over 3,186,538 deaths confirmed as of 2 May 2021, 03:09 pm (WHO website). This situation requires, with utmost, urgency, the development of preventive agents and safe, effective therapies against infection by the causal agent, SARS-CoV-2.

The widespread commitment on the part of the scientific community to develop vaccines to confront this emergency is unprecedented and, as early as November 2020, there were several of them in an advanced stage of experimentation (Stage Ill) (Pfizer/BioNtech, Moderna), as well as many other parallel vaccines in an initial or intermediate stage of development. Notwithstanding the great effort made until now, it is conceivable that COVID-19 will continue to spread globally also in the coming years, with more or less cyclical waves until the circulation of the virus is effectively limited by vaccination or as a result of immunity due to natural infection of the entire population. With such prospects, it is fundamental to develop, in parallel to vaccines, other therapeutic instruments for confronting the next waves of SARS-CoV-2 infections with extreme rapidity.

Among the numerous available therapeutic options, human monoclonal antibodies (mAbs) are the ones that can best meet interests and the narrow timeframes of demand.

In recent years mAbs have demonstrated to be very effective and better tolerated and can be more immediately administered than other types of treatments. In particular, antibodies developed to recognise viral surface proteins have been very useful against infectious diseases such as HIV, Ebola and Middle East respiratory syndrome (MERS) (2, 3, 4).

By virtue of their nature, monoclonal antibodies can provide rapid protection against infections. In fact, once administered, the antibodies promptly enter the bloodstream and offer an immediate protection for several weeks or months. On the other hand, it takes vaccines several weeks to take effect, though they usually provide more long-term protection. Therefore, thanks to their peculiarity, monoclonal antibodies and vaccines could be used together in a complementary manner to contain the pandemic.

Furthermore, mAbs have the potential both to treat infected patients and to prevent infection in healthy individuals. Therefore, antibodies can have prophylactic applications, particularly in relation to some sections of the population, such as the elderly, small children, and immunocompromised persons, who cannot receive a vaccine, or for whom vaccines do not always work with the same efficiency.

For the development of monoclonal antibodies that effectively neutralise SARS-CoV-2 infection, it is of fundamental importance to understand the way how the virus interacts with its target, angiotensin-converting enzyme 2 (hACE2), a protein crucial for the entry of the virus into a cell (5). Viral entry depends on the spike (S) glycoprotein, one of the structural proteins that decorate the surface of SARS-CoV-2. The protein is subdivided into two different subunits, S1 and S2: the former is responsible for binding to ACE2 through the receptor-binding domain (RBD), whilst the latter, the S2 subunit, mediates the fusion of the viral cell membrane to that of the host cell. Given the evident role of “initialising” the viral infection, the S protein has rapidly become the main molecular target to be neutralised with antibodies and the focus of the therapeutic design of vaccines (6).

During the outbreak of the first severe acute respiratory syndrome caused by a coronavirus (SARS-CoV) and the Middle East respiratory syndrome caused by coronavirus (MERS-CoV), plasma derived from convalescent patients was used as an efficient treatment option to reduce the viral load and reduce mortality in severe cases requiring hospitalisation (7, 8). Similarly, in the current COVID-19 pandemic a small number of patients treated with plasma of convalescent patients have shown an evident clinical improvement and a decrease in the viral load (9). However, the administration of purified monoclonal antibodies with neutralising capacity could undoubtedly be a more fruitful and effective treatment strategy, as demonstrated by studies on patients infected by the Ebola virus (10,11).

Preclinical studies on treatments with SARS-CoV-2 infection neutralising antibodies in different animal models, such as mice, hamsters and Rhesus monkeys, have shown promising results with marked reductions of the viral loads in the upper and lower respiratory tracts (12,13,14,15,16,17,18).

Most anti-SARS-CoV-2 antibodies characterised to date have been isolated from individual memory B cells derived from convalescent patients or from animals that are transgenic for the variable regions of human immunoglobulins and immunised against the virus.

However, the antibodies derived from human B cells can show with greater probability a high number of low-affinity mAbs due to a series of factors, such as the viral load or the stage of progression of the infection in the patient from whom they were derived.

To date there have been about 200 research and development programmes focused on monoclonal antibodies against COVID-19; about 80 of them are in phase 1/11/111 of clinical experimentation, 60 in a preclinical phase and 66 in an initial phase of experimentation, with the involvement of about 29 countries and 291 companies and institutions (19) (https://chineseantibody.org/covid-19-track/).

Regeneron Pharmaceuticals Inc (Regeneron), using both B cells derived from convalescent patients and immunised animals, has isolated antibodies that bind in distinct epitopes to the monomeric RBD of the S protein with high affinity, with a dissociation constant ranging from 0.56 to 45.2 nM (20). These antibodies have been tested in assays with viruses and pseudoviruses and show potent neutralisation activity in both (21).

Regeneron also developed two of their best antibodies as a cocktail treatment, REGN-COV2 (REGN10933/casirivimab+REGN10987/imdevimab) and in November 2020 obtained emergency use authorisation (EUA) of the latter from the U.S. Food and Drug Administration (FDA) for the treatment of adult and paediatric COVID-19 patients with intermediate to moderate symptoms. Similarly, the company Eli Lilly, in partnership with AbCellera, using a sample of blood drawn from one of the first U.S. patients who recovered from COVID-19, developed LY-CoV555 (also called LY3819253 or bamlanivimab), likewise authorised in November 2020 for administration as a single intravenous dose in patients with intermediate to moderate symptoms. X-ray crystallography and structural determination by cryo-EM suggest that bamlanivimab binds the S protein RBD in a position overlapping the ACE2 binding site (22) with a K_{D} of 0.071 nM, and blocks S protein-ACE2 interaction with an IC₅₀ value of 0.025 μg/mL (23).

The collaboration between GSK and Vir Biotechnology, on the other hand, has led to the development of VIR-7831/GSK4182136 (24) or Sotrovimab, a monoclonal antibody currently in phase III of clinical experimentation within the framework of the COMET-ICE project; it has demonstrated an ability to neutralise the virus SARS-CoV-2 in vitro by binding to a highly conserved epitope on SARS-CoV-2, shared with SARS-CoV-1.

Lastly, the mAbs currently in phase III of clinical experimentation, such as the antibody CT-P59 of the company Celltrion, include AZD7442, developed by Astrazeneca, which consists in a combination of two monoclonal antibodies (AZD8895/Tixagevimab+AZD1061/Cilgavimab) which have been demonstrated to block the binding of the SARS-CoV-2 virus to host cells and provide protection against infections in cellular and animal models of disease (25).

COVID-19 also causes a hyperinflammatory state that involves several cells and mediators, such as different types of interleukins, tumour necrosis factor, and granulocyte-macrophage colony-stimulating factor and complement (C5, C5a). Considering this deregulation, the existing drugs targeting these mediators have been repurposed for the treatment of COVID-19 (26), with the aim of potentially alleviating the inflammatory symptoms correlated to the infection, rather than acting directly on the virus. For example, the IL-6 inhibitors levilimab, tocilizumab, sarilumab, olokizumab and siltuximab are being tested against COVID-19 (26,27).

The molecular heterogeneity and evolution of SARS-CoV-2 have raised concerns about the extent and effectiveness of protection with specific types of vaccine and the possible escape of the virus from the selective pressure exerted by the immune system. Recent studies have in fact revealed that SARS-CoV-2 has undergone mutations capable of substantially changing its pathogenicity, providing the first concrete evidence that the mutation could influence the severity of the virus that has caused the disease or the damage in the host. The development of drugs and vaccines must thus necessarily take account of the impact of these accumulating mutations.

Various monoclonal antibodies are presently approved for clinical use: Bamlanivimab and Etesevimab (LY-Co555, LY-CoV016) administered in combination, Bebtelovimab (LY-CoV1404), Casirivimab (REGN10933) and Imdevimab (REGN10987), Sotrovimab (VIR-7831), Ticagevimab and Cilgavimab (AZD8895, AZD1061). These antibodies are all monoclonals used for the treatment of COVID-19 infection of medium to moderate severity, both in adults and in children. Despite their initial approval for the treatment of COVID-19, as the mutations occurring in the virus increase and the consequent advent of variants, including the Omicron variant (BA.1), many of these antibodies have proven to have reduced or no neutralising activity against SARS-CoV-2. Updates as of June 2022 confirm only the antibody Bebtelovimab and the combination of antibodies Tixagevimab/Cilgavimab as still authorised for the circulating variants, albeit with variations in the dose administered as regards the latter combination due precisely to the diminished neutralisation vis-á-vis the BA.1 and BA1.1 variants.

In the light of the foregoing, it appears clear that there is need to provide new products, such as monoclonal antibodies, for the prevention and treatment of SARS-CoV-2 infection and COVID-19 disease, which are alternatives to the known products or overcome the disadvantages thereof.

The solution according to the present invention fits into this context; it aims to provide new monoclonal antibodies which, on their own or in combination, can be used for the prevention or treatment of SARS-CoV-2 infection and the COVID-19 disease caused by it.

In particular, according to the present invention, an extensive, variegated library of monoclonal antibodies of medium and high affinity has been generated, from which four mouse monoclonal antibodies have been selected; on the basis of the latter, corresponding humanised antibodies have been prepared which can be advantageously used to prevent SARS-CoV-2 infection or for the treatment of COVID-19.

According to the present invention, the aforesaid antibodies can be used on their own or in combination, with one another or with other monoclonal antibodies, in such a way as to act synergistically on distinct epitopes of the SARS-CoV-2 RBD and combat the development of mutants resistant to treatment. As shown by the experimental data described further below, to obtain the antibodies according to the invention, BALB/C mice and C57BL/6 mice were selected as the primary source of monoclonal antibodies; they were immunised by means of an advantageous immunisation method which provides for the alternating administration of a DNA sequence encoding the full-length spike protein and the RBD protein domain. These animals produce antibody isotopes similar to human ones, including IgA, IgD, IgE, IgG and IgM. The choice to use immunised mice to obtain the antibodies according to the present invention was dictated by the need to obtain an antibody library that was as vast and heterogeneous as possible, wherein the antibodies preferentially had high affinity for the viral target, an objective achievable exclusively through controlled, reproducible hyperimmunisation of the animal.

According to the present invention, among the monoclonal antibodies obtained, the antibodies 3-12B12-F4, 9-7A4-C2, 9-2H7-D7 and 9-8F2-B11 were selected, as they showed to perform particularly well in all the experimental tests conducted and illustrated further below. In particular, the aforementioned antibodies were capable of:
binding, in an equally efficient manner, all the VOCs (variants of concern) or VOIs (variants of interest) that have RBD mutations;
competing for the binding of RBD to ACE2;
having a neutralising action in an assay with pseudoviruses for the D614G, British, South African, Brazilian, Delta and Omicron variants.
inhibiting the SARS-Cov-2 virus with high effectiveness in both the D614G and N501Y variants.

In particular, one of the antibodies of the present invention, the antibody 3-12B12-F4, shows to be neutralising against all the tested variants, including the Omicron variant. This characteristic makes it an excellent candidate, on its own or in combination with the other monoclonal antibodies according to the present invention, for clinical use with the aim of covering as wide a range as possible of viral variants of SARS-CoV-2, present or future.

According to the present invention, the antibodies 9-7A4-C2, 9-2H7-D7 and 9-8F2-B11 were subjected to humanisation. In particular, the mouse antibody was mutagenised to generate a chimeric antibody by substituting the mouse Fc constant region with the human one. Subsequently, the variable regions were humanised by means of single amino acid substitutions in the hypervariable CDRs of the antibody. This process advantageously enables the immunogenicity of the monoclonal antibody to be drastically reduced in order to make it better exploitable for clinical purposes.

According to the present invention, furthermore, a method was developed to actively and effectively induce an immune response against RBD, which comprises the use of a combination of a nucleotide sequence and an amino acid sequence. In particular, according to the present invention use was made of genetic vaccination by means of the plasmid vector pNEB-Ad6-Covid FL, containing optimised spike cDNA (SEQ ID NO:35). This vector was administered intramuscularly and then an electrical field was applied by means of an electroporation apparatus, with the aim of increasing the expression level of the antigen and generate an antibody and/or cell-mediated immune response against the spike protein in the host animal. This genetic vaccination was alternated with classic vaccination by intraperitoneal administration of the recombinant RBD-hFc protein (SEQ ID NO:40), mixed with the Sigma Adjuvant System. Through this combination the production of antibodies against the spike protein in its natural configuration was advantageously induced and a greater antibody response was specifically induced for the RBD portion of the spike protein, which is important for the interaction of the virus with the human ACE2 receptor.

It is therefore a specific object of the present invention relates to a humanised antibody derived from (or obtained starting from) a mouse antibody or the mouse antibody itself, said mouse antibody being selected from:
an antibody (3-12B112-F4) comprising
the variable region of the heavy chain (VH) EVQLQQSGAELVRPGALVKLSCKASGFNIKDYYMHWVKQRPEQGLEWI GWIDPENGNSIYDPKFQGKASITADTSFNTVNLHLSSLTSEDTAVYYCAP YYYDSTYVGTMDYWGQGTSVTVSS (SEQ ID NO:51), and
the variable region of the light chain (VL) QIVLTQSPAIMSASPGEKVTMTCSASSSVGYMYWYQQKPGSSPRLLIYD TSNLASGVPVRFGGSGSGTSYSLTISRMEAEDAATYYCQQWSSYPPRT FGGGTKLEIK (SEQ ID NO:52);
an antibody (9-8F2-B11) comprising
the variable region of the heavy chain (VH) EVQLQQSGAELVKPGASVKLSCTASGFNIKETYVHWVKQRPEQGLEWI GRIDPAIGDSEYDPKFQGKATVTADTSSNTAYLQLSRLTSEDTAVYYCAR TWGPFFDFWGQGTTLTVSS (SEQ ID NO:1), and
the variable region of the light chain (VL) DVVMTQTPLSLPVSLGDQASISCRSSQSLVHSHGNTFLHWYLQKPGQS PKVLIYKVSSRFSGVPDRFSGSGAGTDFTLKISRVEAEDLGVYFCSQSTH VPYTFGGGTKLEIK (SEQ ID NO:2);
an antibody (9-7A4-C2) comprising
the variable region of the heavy chain (VH) EVQLQQSGPELVKPGASVKISCKTSGYTFTEYTMYWVKQSHGKS LEWIGGINPNNGDSSYIQKFKGKATLTVDESSSTAYMELRSLTSEDSAVY YCARDGYPNSYAMDYWGQGTSVTVSS (SEQ ID NO:3), and
the variable region of the light chain (VL)
DIVMSQSPSSLAVSVGDKVTMSCKSSQSLLYSTNQKNYLAWYQ QKPGQSPKLLIYWASTRESGVPDRFTGSGSGTDFTLTIGSVRPEDLAVY YCQQYYSYPWTFGGGTKLEIK (SEQ ID NO:4); and
an antibody (9-2H7-D7) comprising
the variable region of the heavy chain (VH) EVQLQQSGPELVKPGASVTISCKTSGYTFTEYTMYWVKQSHGKSLEWIG GINPYIGDTSYNQNFKGKATLTVDKSSSTAYMELRSLTSEDSAVYYCAR DGYPDYSAMDFWGQGTSVTVSS (SEQ ID NO:5) and
the variable region of the light chain (VL) DIVMSQSPSSLAVSVGEKVTMNCKSSQNLLYSSNQKNYLAWYQQKPG QSPKLLIYWASTRESGVPDRFTGSGSGTDFTLTISSVKAEDLAVYSCQQ YYTYPWTFGGGTKLEIK (SEQ ID NO:6); wherein the CDRs are highlighted in bold.

According to the present invention, the humanised antibody can comprise a VH region comprising a first CDR region GFNIKETY (SEQ ID NO:7), a second CDR region IDPAIGDS (SEQ ID NO:8), and a third CDR region of sequence ARTWGPFFDF (SEQ ID NO:9); and a VL region comprising a first CDR region QSLVHSHGNTF (SEQ ID NO:10), a second CDR region of sequence KVS, and a third CDR region of sequence SQSTHVPYT (SEQ ID NO:11).

According to the present invention, the term “antibody” means a whole monoclonal antibody or a fragment thereof capable of binding the antigen, such as, for example, Fab, Fab′, Fab′-SH, Fv, scFv, (Fab′)2, a double antibody or diabody (dAb), single-domain antibody (sdAb), bispecific antibody, CAR (chimeric antigen receptor) or BiTE (bispecific T-cell engager).

According to a particular embodiment, the humanised antibody according to the present invention can be selected from:
an antibody (humanised 9-8F2-B11) comprising
the VH region
QVQLVQSGAEVKKPGASVKVSCKASGFNIKETYVHWVRQAPGQ GLEWMGRIDPAIGDSEYAQKFQGRVTMTRDTSISTAYMELSRLRSDDTA VYYCARTWGPFFDFWGQGTLVTVSS (SEQ ID NO:12); and
the VL region
DIVMTQTPLSLSVTPGQPASISCKSSQSLLHSHGNTFLHWYLQKP GQSPQVLIYKVSSRFSGVPDRFSGSGSGTDFTLKISRVEAEDVGVYYCS QSTHVPYTFGQGTKLEIK (SEQ ID NO:13);
an antibody (humanised 9-7A4-C2) comprising
the VH region
EVQLQQSGAEVKKPGASVKVSCKTSGYTFTEYTMYWVRQAPGQ RLEWMGGINPNNGDSSYIQKFQGRVTITRDTSASTAYMELSSLRSEDTA VYYCARDGYPNSYAMDYWGQGTLVTVSS (SEQ ID NO:14) and
the VL region
DIVMTQSPDSLAVSLGDRATINCKSSQSLLYSTNQKNYLAWYQQ KPGQPPKLLIYWASTRESGVPDRFSGSGSGTDFTLTISSLQAEDVAVYY CQQYYSYPWTFGQGTKLEIK (SEQ ID NO:15); or
an antibody (humanised 9-2H7-D7) comprising
the VH region
QVQLQQSGAEVKKPGASVKISCKTSGYTFTEYTMYWVKQSHGK SLEWMGGINPYIGDTSYNQNFKGRVTMTRDKSTSTAYMELSSLTSEDSA VYYCARDGYPDYSAMDFWGQGTSVTVSS (SEQ ID NO:16) and
the VL region
DIVMTQSPSSLAVSVGERVTINCKSSQNLLYSSNNKNYLAWYQQ KPGQSPKLLIYWASTRESGVPDRFSGSGSGTDFTLTISSVQAEDVAVYS CQQYYTYPWTFGGGTKLEIK (SEQ ID NO:17),
   wherein the CDRs are highlighted in bold.

The present invention further relates to a nucleotide sequence that encodes for an antibody as defined above.

According to the present invention, the nucleotide sequence encoding for SEQ ID NO:51 can be:

GAGGTGCAGCTGCAGCAGAGCGGCGCCGAGCTGGTGAGGCCCGGC GCCCTGGTGAAGCTGAGCTGCAAGGCCAGCGGCTTCAACATCAAGG ACTACTACATGCACTGGGTGAAGCAGAGGCCCGAGCAGGGCCTGGA GTGGATCGGCTGGATCGACCCCGAGAACGGCAACAGCATCTACGA CCCCAAGTTCCAGGGCAAGGCCAGCATCACCGCCGACACCAGCTTC AACACCGTGAACCTGCACCTGAGCAGCCTGACCAGCGAGGACACCG CCGTGTACTACTGCGCCCCCTACTACTACGACAGCACCTACGTGGG CACCATGGACTACTGGGGCCAGGGCACCAGCGTGACCGTGAGCAG C (SEQ ID NO:53);
and the nucleotide sequence encoding for SEQ ID NO:52 can be:
   CAGATCGTGCTGACCCAGAGCCCCGCCATCATGAGCGCCAGCCCCG GCGAGAAGGTGACCATGACCTGCAGCGCCAGCAGCAGCGTGGGCT ACATGTACTGGTACCAGCAGAAGCCCGGCAGCAGCCCCAGGCTGCT GATCTACGACACCAGCAACCTGGCCAGCGGCGTGCCCGTGAGGTTC GGCGGCAGCGGCAGCGGCACCAGCTACAGCCTGACCATCAGCAGG ATGGAGGCCGAGGACGCCGCCACCTACTACTGCCAGCAGTGGAGC AGCTACCCCCCCAGGACCTTCGGCGGCGGCACCAAGCTGGAGATCA AG (SEQ ID NO:54);
the nucleotide sequence encoding for SEQ ID NO:1 can be:

GAGGTGCAGCTGCAGCAGAGCGGCGCCGAGCTGGTGAAGCCCGGC GCCAGCGTGAAGCTGAGCTGCACCGCCAGCGGCTTCAACATCAAGG AGACCTACGTGCACTGGGTGAAGCAGAGGCCCGAGCAGGGCCTGG AGTGGATCGGCAGGATCGACCCCGCCATCGGCGACAGCGAGTACG ACCCCAAGTTCCAGGGCAAGGCCACCGTGACCGCCGACACCAGCAG CAACACCGCCTACCTGCAGCTGAGCAGGCTGACCAGCGAGGACACC GCCGTGTACTACTGCGCCAGGACCTGGGGCCCCTTCTTCGACTTCT GGGGCCAGGGCACCACCCTGACCGTGAGCAGC (SEQ ID NO:18);
the nucleotide sequence encoding for SEQ ID NO:2 can be:
   GACGTGGTGATGACCCAGACCCCCCTGAGCCTGCCCGTGAGCCTGG GCGACCAGGCCAGCATCAGCTGCAGGAGCAGCCAGAGCCTGGTGC ACAGCCACGGCAACACCTTCCTGCACTGGTACCTGCAGAAGCCCGG CCAGAGCCCCAAGGTGCTGATCTACAAGGTGAGCAGCAGGTTCAGC GGCGTGCCCGACAGGTTCAGCGGCAGCGGCGCCGGCACCGACTTC ACCCTGAAGATCAGCAGGGTGGAGGCCGAGGACCTGGGCGTGTACT TCTGCAGCCAGAGCACCCACGTGCCCTACACCTTCGGCGGCGGCA CCAAGCTGGAGATCAAG (SEQ ID NO:19);
the nucleotide sequence encoding for SEQ ID NO:3 is
GAGGTGCAGCTGCAGCAGTCTGGACCAGAGCTGGTGAAGCCT GGAGCATCCGTGAAGATCTCTTGCAAGACCAGCGGCTATACCTTCAC AGAGTACACAATGTATTGGGTGAAGCAGTCCCACGGCAAGTCTCTG GAGTGGATCGGCGGCATCAACCCAAACAATGGCGATAGCTCCTATA TCCAGAAGTTTAAGGGCAAGGCCACCCTGACAGTGGACGAGTCTAG CTCCACCGCCTACATGGAGCTGCGGTCCCTGACATCTGAGGACAGC GCCGTGTACTATTGTGCCAGAGATGGCTACCCCAATAGCTATGCCA TGGACTACTGGGGCCAGGGCACCTCCGTGACAGTGTCTAGC (SEQ ID NO:20) and
the nucleotide sequence encoding for SEQ ID NO:4 is
GACATCGTGATGTCCCAGTCTCCTAGCTCCCTGGCCGTGAGC GTGGGCGATAAGGTGACCATGTCCTGCAAGTCTAGCCAGTCTCTGCT GTACAGCACAAACCAGAAGAATTACCTGGCCTGGTATCAGCAGAAG CCCGGCCAGAGCCCTAAGCTGCTGATCTATTGGGCATCCACCAGGG AGTCTGGAGTGCCAGACAGATTCACCGGCAGCGGCTCCGGAACAGA CTTCACCCTGACAATCGGCTCTGTGCGGCCAGAGGACCTGGCCGTG TACTATTGTCAGCAGTACTATTCCTACCCCTGGACCTTCGGCGGCGG CACAAAGCTGGAGATCAAG (SEQ ID NO:21);
the nucleotide sequence encoding for SEQ ID NO:5 is
GAAGTGCAACTACAACAAAGTGGTCCAGAACTGGTGAAGCCTG GCGCTTCTGTGACCATCTCCTGCAAGACCTCTGGCTACACCTTTACC GAGTACACCATGTACTGGGTCAAGCAGTCTCACGGAAAATCTCTGGA GTGGATCGGCGGCATCAACCCCTACATCGGCGACACCTCCTACAAC CAGAACTTCAAGGGCAAGGCCACCCTGACCGTGGACAAGTCCTCCT CTACCGCCTACATGGAACTGCGGAGCCTGACATCCGAGGATTCTGCT GTGTACTACTGTGCCAGAGATGGATATCCTGACTACTCCGCCATGG ACTTCTGGGGCCAGGGCACCTCCGTGACAGTGTCCAGC (SEQ ID NO:22) and
the nucleotide sequence encoding for SEQ ID NO:6 is GATATCGTGATGAGTCAAAGTCCAAGTAGTCTCGCCGTGTCCGTGGG AGAGAAGGTGACCATGAACTGCAAGTCCTCTCAGAATCTGCTGTACT CCTCCAACCAGAAGAACTACCTGGCCTGGTACCAGCAAAAACCTGG CCAGTCTCCTAAGCTGCTGATCTACTGGGCTTCTACCAGAGAGTCTG GCGTGCCCGATCGGTTTACCGGCTCCGGCTCTGGCACCGACTTCAC ACTGACCATCTCCAGCGTCAAGGCCGAGGACCTGGCTGTGTACAGC TGTCAGCAGTACTACACCTATCCTTGGACCTTCGGCGGCGGAACAA AGCTGGAAATCAAG (SEQ ID NO:23); wherein the CDR regions are highlighted in bold.

According to the present invention, the nucleotide sequence encoding for SEQ ID NO:7 is GGCTTCAACATCAAGGAGACCTAC (SEQ ID NO:24), the nucleotide sequence encoding for SEQ ID NO: 8 is ATCGACCCCGCCATCGGCGACAGC (SEQ ID NO:25), the nucleotide sequence encoding for SEQ ID NO:9 is GCCAGGACCTGGGGCCCCTTCTTCGACTTC (SEQ ID NO:26), the nucleotide sequence encoding for SEQ ID NO:10 is CAGAGCCTGGTGCACAGCCACGGCAACACCTTC (SEQ ID NO:27), the nucleotide sequence encoding for KVS is AAGGTGAGC and the nucleotide sequence encoding for SEQ ID NO:11 is AGCCAGAGCACCCACGTGCCCTACACC (SEQ ID NO:28).

According to the present invention, the nucleotide sequence encoding for SEQ ID NO:12 is CAGGTGCAGCTGGTGCAGAGCGGCGCCGAGGTGAAGAAGCCCGGC GCCAGCGTGAAGGTGAGCTGCAAGGCCAGCGGCTTCAACATCAAGG AGACCTACGTGCACTGGGTGAGGCAGGCCCCCGGCCAGGGCCTGG AGTGGATGGGCAGGATCGACCCCGCCATCGGCGACAGCGAGTACG CCCAGAAGTTCCAGGGCAGGGTGACCATGACCAGGGACACCAGCAT CAGCACCGCCTACATGGAGCTGAGCAGGCTGAGGAGCGACGACACC GCCGTGTACTACTGCGCCAGGACCTGGGGCCCCTTCTTCGACTTCT GGGGCCAGGGCACCCTGGTGACCGTGAGCAGC (SEQ ID NO:29);
the nucleotide sequence encoding for SEQ ID NO:13 is
GACATCGTGATGACCCAGACCCCCCTGAGCCTGAGCGTGACC CCCGGCCAGCCCGCCAGCATCAGCTGCAAGAGCAGCCAGAGCCTG CTGCACAGCCACGGCAACACCTTCCTGCACTGGTACCTGCAGAAGC CCGGCCAGAGCCCCCAGGTGCTGATCTACAAGGTGAGCAGCAGGTT CAGCGGCGTGCCCGACAGGTTCAGCGGCAGCGGCAGCGGCACCGA CTTCACCCTGAAGATCAGCAGGGTGGAGGCCGAGGACGTGGGCGTG TACTACTGCAGCCAGAGCACCCACGTGCCCTACACCTTCGGCCAGG GCACCAAGCTGGAGATCAAG (SEQ ID NO:30);
the nucleotide sequence encoding for SEQ ID NO:14 is
GAGGTGCAGCTGCAGCAGAGCGGCGCCGAGGTGAAGAAGCC CGGCGCCAGCGTGAAGGTGAGCTGCAAGACCAGCGGCTACACCTTC ACCGAGTACACCATGTACTGGGTGAGGCAGGCCCCCGGCCAGAGG CTGGAGTGGATGGGCGGCATCAACCCCAACAACGGCGACAGCAGC TACATCCAGAAGTTCCAGGGCAGGGTGACCATCACCAGGGACACCA GCGCCAGCACCGCCTACATGGAGCTGAGCAGCCTGAGGAGCGAGG ACACCGCCGTGTACTACTGCGCCAGGGACGGCTACCCCAACAGCTA CGCCATGGACTACTGGGGCCAGGGCACCCTGGTGACCGTGAGCAG C (SEQ ID NO:31);
the nucleotide sequence encoding for SEQ ID NO:15 is
GACATCGTGATGACCCAGAGCCCCGACAGCCTGGCCGTGAGC CTGGGCGACAGGGCCACCATCAACTGCAAGAGCAGCCAGAGCCTGC TGTACAGCACCAACCAGAAGAACTACCTGGCCTGGTACCAGCAGAA GCCCGGCCAGCCCCCCAAGCTGCTGATCTACTGGGCCAGCACCAGG GAGAGCGGCGTGCCCGACAGGTTCAGCGGCAGCGGCAGCGGCACC GACTTCACCCTGACCATCAGCAGCCTGCAGGCCGAGGACGTGGCCG TGTACTACTGCCAGCAGTACTACAGCTACCCCTGGACCTTCGGCCA GGGCACCAAGCTGGAGATCAAG (SEQ ID NO:32);
the nucleotide sequence encoding for SEQ ID NO:16 is
CAGGTGCAGCTGCAGCAGAGCGGCGCCGAGGTGAAGAAGCC CGGCGCCAGCGTGAAGATCAGCTGCAAGACCAGCGGCTACACCTTC ACCGAGTACACCATGTACTGGGTGAAGCAGAGCCACGGCAAGAGCC TGGAGTGGATGGGCGGCATCAACCCCTACATCGGCGACACCAGCTA CAACCAGAACTTCAAGGGCAGGGTGACCATGACCAGGGACAAGAGC ACCAGCACCGCCTACATGGAGCTGAGCAGCCTGACCAGCGAGGACA GCGCCGTGTACTACTGCGCCAGGGACGGCTACCCCGACTACAGCG CCATGGACTTCTGGGGCCAGGGCACCAGCGTGACCGTGAGCAGC (SEQ ID NO:33) and
the nucleotide sequence encoding for SEQ ID NO:17 is
GACATCGTGATGACCCAGAGCCCCAGCAGCCTGGCCGTGAGC GTGGGCGAGAGGGTGACCATCAACTGCAAGAGCAGCCAGAACCTGC TGTACAGCAGCAACAACAAGAACTACCTGGCCTGGTACCAGCAGAA GCCCGGCCAGAGCCCCAAGCTGCTGATCTACTGGGCCAGCACCAGG GAGAGCGGCGTGCCCGACAGGTTCAGCGGCAGCGGCAGCGGCACC GACTTCACCCTGACCATCAGCAGCGTGCAGGCCGAGGACGTGGCCG TGTACAGCTGCCAGCAGTACTACACCTACCCCTGGACCTTCGGCGG CGGCACCAAGCTGGAGATCAAG (SEQ ID NO:34); wherein the CDR regions are highlighted in bold.

The present invention also relates to an expression vector comprising a nucleotide sequence as defined above.

According to the present invention, the vector can be selected in the group consisting of a plasmid, for example bacterial plasmids, an RNA, an RNA that replicates, amplicons obtained by PCR, a viral vector such as, for example, adenovirus, poxvirus, vaccinia virus, fowlpox, herpes virus, adeno-associated virus (AAV), alphavirus, lentivirus, lambda phage, lymphocytic choriomeningitis virus, Listeria sp, Salmonella sp.

The present invention further relates to a cell comprising an expression vector as defined above.

The present invention also relates to a pharmaceutical composition comprising one or more antibodies, preferably one or more humanised antibodies, as defined above, one or more nucleotide sequences as defined above, one or more vectors as defined above, or one or more cells as defined above, together with one or more pharmaceutically acceptable excipients and/or adjuvants. Therefore, when the antibody is a humanised antibody, said nucleotide sequence encodes for said humanised antibody and said vector or cell comprises said nucleotide sequence encoding for said humanised antibody.

In particular, an antibody, a nucleotide sequence, a vector or a cell according to the present invention can be included in a pharmaceutical or diagnostic composition. The composition can comprise pharmaceutically acceptable vehicles and excipients, such as, for example, diluents, adjuvants, buffer agents, emulsifiers, humectants, solubilisation agents or other substances that enable or facilitate its administration, nebulisation, distribution in the body and delivery to the site of action of the antibody, or reduce its toxicity, increase its bioavailability, or favour the compliance of the individual to whom it is administered. For the choice of a suitable excipient for the applications envisaged herein, one may refer to the Handbook of Pharmaceutical Excipients, 5th Edition, R. C. Rowe; P. J. Seskey and S. C. Owen, Pharmaceutical Press, London, Chicago. The pharmaceutical composition can be in the form of a solution, suspension, emulsion, tablet, capsule, microcapsule, liposome, powder, extended-release formulation or lyophilisate.

According to the present invention, when the pharmaceutical composition of the present invention comprises more antibodies, nucleotide sequences that encoding for said more antibodies, vectors that comprise said nucleotide sequences or cells that comprise said vectors, they can consist in the following alternative combinations:
a combination of four antibodies, wherein a first humanised antibody 3-12B12-F4 is obtained from the mouse antibody having the VH sequence SEQ ID NO:51 and the VL sequence SEQ ID NO:52, the second humanised antibody 9-8F2-B11 comprises the VH sequence SEQ ID NO:12 and the VL sequence SEQ ID NO:13, the third humanised antibody 9-7A4-C2 comprises the VH sequence SEQ ID NO:14 and the VL sequence SEQ ID NO:15, and the fourth humanised antibody 9-2H7-D7 comprises the VH sequence SEQ ID NO:16 and the VL sequence SEQ ID NO:17;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-8F2-B11 comprises VH the sequence SEQ ID NO:12 and the VL sequence SEQ ID NO:13, and a third humanised antibody 9-7A4-C2 comprises the VH sequence SEQ ID NO:14 and the VL sequence SEQ ID NO:15;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-8F2-B11 comprises the VH sequence SEQ ID NO:12 and the VL sequence SEQ ID NO:13, and a third humanised antibody 9-2H7-D7 comprises the VH sequence SEQ ID NO:16 and the VL sequence SEQ ID NO:17;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-7A4-C2 comprises the VH sequence SEQ ID NO:14 and the VL sequence SEQ ID NO:15, and a third humanised antibody 9-2H7-D7 comprises the VH sequence SEQ ID NO:16 and the VL sequence SEQ ID NO:17;
a combination of three antibodies, wherein a first humanised antibody 9-8F2-B11 comprises the VH sequence SEQ ID NO:12 and the VL sequence SEQ ID NO:13, a second humanised antibody 9-7A4-C2 comprises the VH sequence SEQ ID NO:14 and the VL sequence SEQ ID NO:15, and a third humanised antibody 9-2H7-D7 comprises the VH sequence SEQ ID NO:16 and the VL sequence SEQ ID NO:17;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-8F2-B11 comprises the VH sequence SEQ ID NO:12 and the VL sequence SEQ ID NO:13;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-7A4-C2 comprises the VH sequence SEQ ID NO:14 and the VL sequence SEQ ID NO:15;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-2H7-D7 comprises the VH sequence SEQ ID NO:16 and the VL sequence SEQ ID NO:17;
a combination of two antibodies, wherein a first humanised antibody 9-8F2-B111 comprises the VH sequence SEQ ID NO:12 and the VL sequence SEQ ID NO:13, and a second humanised antibody 9-7A4-C2 comprises the VH sequence SEQ ID NO:14 and the VL sequence SEQ ID NO:15;
a combination of two antibodies, wherein a first humanised antibody 9-8F2-B111 comprises the VH sequence SEQ ID NO:12 and the VL sequence SEQ ID NO:13, and a second humanised antibody 9-2H7-D7 comprises the VH sequence SEQ ID NO:16 and the VL sequence SEQ ID NO:17;
a combination of two antibodies, wherein a first humanised antibody 9-7A4-C2 comprises the VH sequence SEQ ID NO:14 and the VL sequence SEQ ID NO:15, and a second humanised antibody 9-2H7-D7 comprises the VH sequence SEQ ID NO:16 and the VL sequence SEQ ID NO:17.

As mentioned above, the CDR regions of each VH and VL are highlighted in bold. The composition according to the present invention can thus comprise one of the antibodies according to the invention, a nucleotide sequence encoding for said antibody, a vector that comprises the nucleotide sequence or a cell comprising said vector or combinations of said antibodies, combinations of the sequences encoding for said antibodies, combinations of vectors that comprise said nucleotide sequences or of cells that comprise said vectors. The combination can be selected from the following combinations:
a combination of four antibodies, wherein a first humanised antibody 3-12B12-F4 is obtained from the mouse antibody having the VH sequence SEQ ID NO:51 and the VL sequence SEQ ID NO:52, the second humanised antibody 9-8F2-B11 comprises the VH sequence SEQ ID NO:12 and the VL sequence SEQ ID NO:13, the third humanised antibody 9-7A4-C2 comprises the VH sequence SEQ ID NO:14 and the VL sequence SEQ ID NO:15, and the fourth humanised antibody 9-2H7-D7 comprises the VH sequence SEQ ID NO:16 and the VL sequence SEQ ID NO:17;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-8F2-B11 comprises the VH sequence SEQ ID NO:12 and the VL sequence SEQ ID NO:13, and a third humanised antibody 9-7A4-C2 comprises the VH sequence SEQ ID NO:14 and the VL sequence SEQ ID NO:15;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-8F2-B11 comprises the VH sequence SEQ ID NO:12 and the VL sequence SEQ ID NO:13, and a third humanised antibody 9-2H7-D7 comprises the VH sequence SEQ ID NO:16 and the VL sequence SEQ ID NO:17;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-7A4-C2 comprises the VH sequence SEQ ID NO:14 and the VL sequence SEQ ID NO:15, and a third humanised antibody 9-2H7-D7 comprises the VH sequence SEQ ID NO:16 and the VL sequence SEQ ID NO:17;
a combination of three antibodies, wherein a first humanised antibody 9-8F2-B11 comprises the VH sequence SEQ ID NO:12 and the VL sequence SEQ ID NO:13, a second humanised antibody 9-7A4-C2 comprises the VH sequence SEQ ID NO:14 and the VL sequence SEQ ID NO:15, and a third humanised antibody 9-2H7-D7 comprises the VH sequence SEQ ID NO:16 and the VL sequence SEQ ID NO:17;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-8F2-B11 comprises the VH sequence SEQ ID NO:12 and the VL sequence SEQ ID NO:13;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-7A4-C2 comprises the VH sequence SEQ ID NO:14 and the VL sequence SEQ ID NO:15;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-2H7-D7 comprises the VH sequence SEQ ID NO:16 and the VL sequence SEQ ID NO:17;
a combination of two antibodies, wherein a first humanised antibody 9-8F2-B111 comprises the VH sequence SEQ ID NO:12 and the VL sequence SEQ ID NO:13, and a second humanised antibody 9-7A4-C2 comprises the VH sequence SEQ ID NO:14 and the VL sequence SEQ ID NO:15;
a combination of two antibodies, wherein a first humanised antibody 9-8F2-B111 comprises the VH sequence SEQ ID NO:12 and the VL sequence SEQ ID NO:13, and a second humanised antibody 9-2H7-D7 comprises the VH sequence SEQ ID NO:16 and the VL sequence SEQ ID NO:17;
a combination of two antibodies, wherein a first humanised antibody 9-7A4-C2 comprises the VH sequence SEQ ID NO:14 and the VL sequence SEQ ID NO:15, and a second humanised antibody 9-2H7-D7 comprises the VH sequence SEQ ID NO:16 and the VL sequence SEQ ID NO:17.

Alternatively, the combinations can comprise nucleotide sequences encoding for the sequences just described, the vectors comprising said nucleotide sequences or the cells that comprise said vectors.

The present invention further relates to an antibody as defined above, a nucleotide sequence as defined above, a vector as defined above, a cell as defined above or a pharmaceutical composition as defined above, for use in the medical field.

Furthermore, the present invention relates to an antibody as defined above, a nucleotide sequence as defined above, a vector as defined above, a cell as defined above or a pharmaceutical composition as defined above, for use in the prevention and treatment of SARS-CoV-2 infection and COVID-19 disease.

According to the present invention, said antibody, nucleotide sequence, vector, cell or pharmaceutical composition, for the above-mentioned use, can be administered by oral, sublingual, nasal, parenteral, intravenous, subcutaneous, intramuscular, intradermal, or intrathecal route.

The subject matter of the present invention further relates to an in vitro diagnostic method for detecting SARS-CoV-2 by using an antibody as defined above.

The present invention further relates to a combination of two, three, or four
mouse or humanised antibodies as defined above,
nucleotide sequences encoding for said mouse or humanised antibodies as defined above,
vectors that comprise said nucleotide sequences as defined above or
cells that comprise said vectors as defined above,
for separate or sequential use in the prevention and treatment of SARS-CoV-2 infection and COVID-19 disease.

Therefore, the combinations according to the present invention, for the use specified above, can comprise or consist, for example, in the following combinations:
a combination of four antibodies, wherein a first humanised antibody 3-12B12-F4 is obtained from the mouse antibody having the VH sequence SEQ ID NO:51 and the VL sequence SEQ ID NO:52, the second humanised antibody 9-8F2-B11 comprises the VH sequence SEQ ID NO:12 and the VL sequence SEQ ID NO:13, the third humanised antibody 9-7A4-C2 comprises the VH sequence SEQ ID NO:14 and the VL sequence SEQ ID NO:15, and the fourth humanised antibody 9-2H7-D7 comprises the VH sequence SEQ ID NO:16 and the VL sequence SEQ ID NO:17;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-8F2-B11 comprises the VH sequence SEQ ID NO:12 and the VL sequence SEQ ID NO:13, and a third humanised antibody 9-7A4-C2 comprises the VH sequence SEQ ID NO:14 and the VL sequence SEQ ID NO:15;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-8F2-B11 comprises the VH sequence SEQ ID NO:12 and the VL sequence SEQ ID NO:13, and a third humanised antibody 9-2H7-D7 comprises the VH sequence SEQ ID NO:16 and the VL sequence SEQ ID NO:17;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-7A4-C2 comprises the VH sequence SEQ ID NO:14 and the VL sequence SEQ ID NO:15, and a third humanised antibody 9-2H7-D7 comprises the VH sequence SEQ ID NO:16 and the VL sequence SEQ ID NO:17;
a combination of three antibodies, wherein a first humanised antibody 9-8F2-B11 comprises the VH sequence SEQ ID NO:12 and the VL sequence SEQ ID NO:13, a second humanised antibody 9-7A4-C2 comprises the VH sequence SEQ ID NO:14 and the VL sequence SEQ ID NO:15, and a third humanised antibody 9-2H7-D7 comprises the VH sequence SEQ ID NO:16 and the VL sequence SEQ ID NO:17;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-8F2-B11 comprises the VH sequence SEQ ID NO:12 and the VL sequence SEQ ID NO:13;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-7A4-C2 comprises the VH sequence SEQ ID NO:14 and the VL sequence SEQ ID NO:15;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-2H7-D7 comprises the VH sequence SEQ ID NO:16 and the VL sequence SEQ ID NO:17;
a combination of two antibodies, wherein a first humanised antibody 9-8F2-B111 comprises the VH sequence SEQ ID NO:12 and the VL sequence SEQ ID NO:13, and a second humanised antibody 9-7A4-C2 comprises the VH sequence SEQ ID NO:14 and the VL sequence SEQ ID NO:15;
a combination of two antibodies, wherein a first humanised antibody 9-8F2-B111 comprises the VH sequence SEQ ID NO:12 and the VL sequence SEQ ID NO:13, and a second humanised antibody 9-2H7-D7 comprises the VH sequence SEQ ID NO:16 and the VL sequence SEQ ID NO:17;
a combination of two antibodies, wherein a first humanised antibody 9-7A4-C2 comprises the VH sequence SEQ ID NO:14 and the VL sequence SEQ ID NO:15, and a second humanised antibody 9-2H7-D7 comprises the VH sequence SEQ ID NO:16 and the VL sequence SEQ ID NO:17.

“Separate use” means the administration, at the same time, of the compounds of the combination according to the invention in pharmaceutically distinct forms.

“Sequential use” means the successive administration of the compounds of the combination according to the invention, each in a distinct pharmaceutical form.

According to the present invention, the components of the combination of the invention for the above-mentioned use can be administered by the oral, sublingual, nasal, parenteral, intravenous, subcutaneous, intramuscular, intradermal, or intrathecal route.

The present invention further relates to the use of an antibody as defined in claim 1 for the preparation of a humanised antibody.

As mentioned above, the antibodies 3-12B12-F4, 9-8F2-B11, 9-2H7-D7 and 9-7A4-C2 according to the present invention can be used in the humanised version thereof, i.e. they can comprise sequences or amino acid residues of human and non-human origin. For example, the humanised antibody can comprise one or both variable domains in which all the hypervariable regions (CDRs) correspond to those of a non-human antibody, whereas the framework regions correspond to those of a human antibody. The humanised antibody can comprise at least a portion of a constant region of human derivation. In general, “humanised” form of an antibody refers to an antibody that was subjected to a humanisation process. The methods for humanising an antibody are known in the art—see for example Almagro J. C. and Fransson J., (2008) Frontiers in Bioscience 13: 1619-1633—and are based on methods such as “CDR-grafting”, “Resurfacing, “Superhumanization” and “Human String Content Optimization”.

According to the present invention, furthermore, the regions of the heavy chain of an antibody according to the invention can also be modified to reduce binding to the human Fc-gamma receptor to eliminate a possible “ADE” (antibody-dependent enhancement) effect, such as, for example, the mutation L234A, L235A (28) or further stimulation of the immune system by ADCC (antibody-dependent cellular cytotoxicity), as in the case, for example, of mutations S298A, E333A, K334A (29) or CDC (complement-dependent cytotoxicity), as in the case, for example, of the mutations S267E, H268F, S324T (30). Furthermore, the constant region of the heavy chain can be modified to increase affinity for the human neonatal Fc receptor in order to increase the half-life, as in the case, for example, of the mutation N434A_(31).

As mentioned above, the antibodies according to the present invention can be used as fragments selected from Fab, Fab′, Fab′-SH, Fv, scFv, (Fab′)2, diabodies (dAb) and single-domain antibodies (sdAb) and combined to generate bispecific antibodies. Furthermore, the scFv fragments can be used to generate CAR-T or combined with antibodies and fragments that bind the human CD3 to generate BiTEs (bispecific T-cell engagers).

Furthermore, the antibodies according to the invention can be used to generate a conjugated molecule containing an antibody according to the present invention covalently bound to a compound selected from an isotopic or fluorescent marker, a toxin, an enzyme, or a drug.

The antibodies according to the present invention, besides being able to be administered in purified protein form, can be delivered through an expression vector containing a nucleic acid encoding the antibody, or parts or fragments thereof, for example by means of lipid nanoparticles or another method of expression vector delivery or by electroporation.

The present invention further relates to a combination of a nucleotide sequence comprising or consisting of SEQ ID NO:35 (coding for the SARS-CoV-2 spike protein), or a nucleotide sequence having a sequence identity to SEQ ID NO:35 of at least 96%, at least 97%, at least 98% or at least 99%, or a vector comprising said nucleotide sequence or a cell comprising said vector, with an amino acid sequence comprising or consisting of SEQ ID NO:36 (i.e. the RBD of the SARS-CoV-2 spike protein), or an amino acid sequence having a sequence identity to SEQ ID NO:36 of at least 96%, at least 97%, at least 98% or at least 99%, for simultaneous, separate or sequential use in the generation (preparation) of antibodies against SARS-CoV-2 in a human or animal body, i.e. in the immunisation or vaccination of said human or animal body.

“Simultaneous use” means the administration of the two compounds of the combination according to the invention in a single identical pharmaceutical composition.

“Separate use” means the administration, at the same time, of the two compounds of the combination according to the invention in pharmaceutically distinct forms.

“Sequential use” means the successive administration of the two compounds of the combination according to the invention, each in a distinct pharmaceutical form.

According to the present invention, said amino acid sequence of the above-mentioned combination for the use specified above can further comprise one or more leader sequences, such as, for example, the leader sequence of the tissue plasminogen activator (TPA), of IgK, of growth hormone, of serum albumin or of alkaline phosphatase and/or one or more immunomodulatory amino acid sequences, such as, for example, the crystallisable fragment (Fc), Toxoplasma Gondii profilin-like protein (PFTG) or a functional fragment derived therefrom, the B subunit of Escherichia Coli heat-labile toxin (LTB) or the tetanus toxin (TT).

For example, the amino acid sequence can be IgK-RBD-Fc. The amino acid sequence can also comprise one or more linker sequences. The use of leader sequences and immunomodulatory sequences provides the technical effect of improving the antibody titre. In particular, the leader sequence has the function of transporting antigens outside the cells of the organism transfected with the vector or plasmid, for example by electroporation. The leader sequence increases protein secretion, whereas the immunomodulatory sequences stimulate the immune system to produce antibodies.

In reference to the combination of the present invention for the use specified above, said amino acid sequence can further comprise the IgK leader sequence, or the IgK chain signal peptide, of sequence SEQ ID NO:37 and the Fc domain of sequence SEQ ID NO:38. The IgK leader sequence, or IgK chain signal peptide, of sequence SEQ ID NO:37, is located at the N-terminus of said sequence SEQ ID NO:36, whilst the Fc domain of sequence SEQ ID NO:38 can be located at the C-terminus of said sequence SEQ ID NO:36, or else it can also be inserted between IgK and RBD, thus in the C-terminal portion.

According to the present invention, said nucleotide sequence comprising or consisting of SEQ ID NO:35, or said vector or said cell, of the combination of the invention for the use specified above, can be administered before said amino acid sequence comprising or consisting of SEQ ID NO:36.

Again in reference to the combination of the invention for the use specified above, according to one embodiment, a first dose of said nucleotide sequence comprising or consisting of SEQ ID NO:35 or of said vector or of said cell is administered, subsequently a first dose of said amino acid sequence comprising or consisting of SEQ ID NO:36 is administered, subsequently a second dose of said nucleotide sequence comprising or consisting of SEQ ID NO:35 or of said vector or of said cell is administered, and subsequently a second dose of said amino acid sequence comprising or consisting of SEQ ID NO:36 is administered, each dose preferably being administered a week after the previously administered dose.

According to the present invention, said nucleotide sequence, or said vector or said cell, and said amino acid sequence, can be administered through oral, sublingual, nasal, parenteral, or intravenous route.

The present invention also relates to a method for the production of antibodies against SARS-CoV-2 in a human or animal body, i.e. for the immunisation or vaccination of a human or animal body, said method comprising administering simultaneously, separately or sequentially
a nucleotide sequence comprising or consisting of SEQ ID NO:35 (encoding the SARS-CoV-2 spike protein), or a nucleotide sequence having a sequence identity to SEQ ID NO:35 of at least 96%, at least 97%, at least 98% or at least 99%, or an expression vector comprising said nucleotide sequence or cell comprising said vector, and
an amino acid sequence comprising or consisting of SEQ ID NO:36 (i.e. the RBD of the SARS-CoV-2 spike protein), or an amino acid sequence having a sequence identity to SEQ ID NO:36 of at least 96%, at least 97%, at least 98% or at least 99%.

“Administering simultaneously” means the administration of the two compounds according to the invention in a single and identical pharmaceutical composition.

“Administering separately” means the administration, at the same time, of the two compounds according to the invention in pharmaceutically distinct forms.

“Administering sequentially” means the successive administration of the two compounds according to the invention, each in a distinct pharmaceutical form.

Said amino acid sequence can further comprise one or more leader sequences, such as, for example, the leader sequence of the tissue plasminogen activator (TPA), of IgK, of growth hormone, of serum albumin or of alkaline phosphatase and/or one or more immunomodulatory amino acid sequences, such as, for example, the crystallisable fragment (Fc), Toxoplasma Gondii profilin-like protein (PFTG) or a functional fragment derived therefrom, the B subunit of Escherichia Coli heat-labile toxin (LTB) or the tetanus toxin (TT).

For example, the amino acid sequence can be IgK-RBD-Fc. The amino acid sequence can also comprise one or more linker sequences.

In particular, according to a preferred embodiment, said amino acid sequence can further comprise the IgK leader sequence, or the IgK chain signal peptide, of sequence SEQ ID NO:37, located at the N-terminus of said sequence SEQ ID NO:36, and the Fc domain of sequence SEQ ID NO:38 located at the C-terminus of said sequence SEQ ID NO:36.

According to the method of the invention, the nucleotide sequence comprising or consisting of SEQ ID NO:35 can be administered before the amino acid sequence comprising or consisting of SEQ ID NO:36. In particular, according to one embodiment of the method of the invention, a first dose of said nucleotide sequence comprising or consisting of SEQ ID NO:35 or of said vector or of said cell is administered, subsequently a first dose of said amino acid sequence comprising or consisting of SEQ ID NO:36 is administered, subsequently a second dose of said nucleotide sequence comprising or consisting of SEQ ID NO:35 or of said vector or of said cell is administered, and subsequently a second dose of said amino acid sequence comprising or consisting of SEQ ID NO:36 is administered, each dose preferably being administered a week after the previously administered dose.

The present invention will now be described, by way of illustration but not limitation, according to a preferred embodiment thereof, with specific reference to the examples and the figures of the appended drawings, in which:

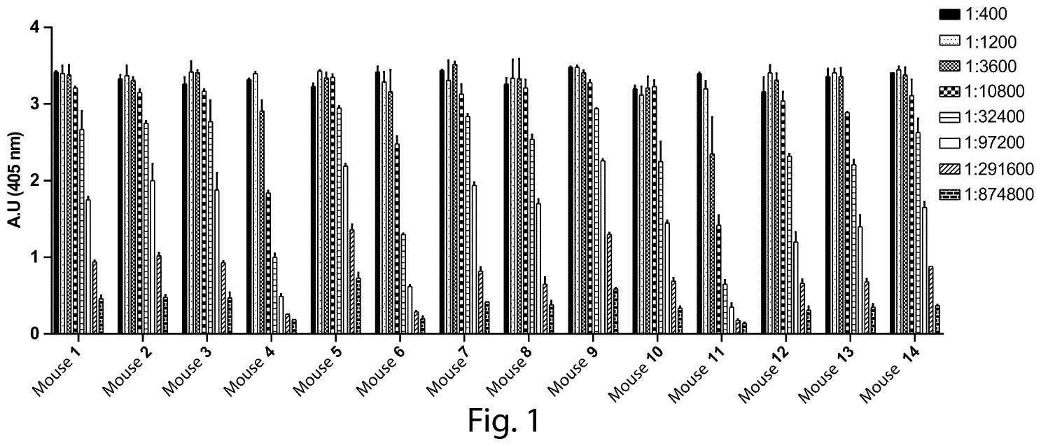

FIG. 1 shows the antibody titre; in particular, the graph shows the mean values±SD of absorbance (405 nm), obtained by ELISA for the RBD WT protein, of the sera of animals immunised with scalar dilutions starting from 1:400;

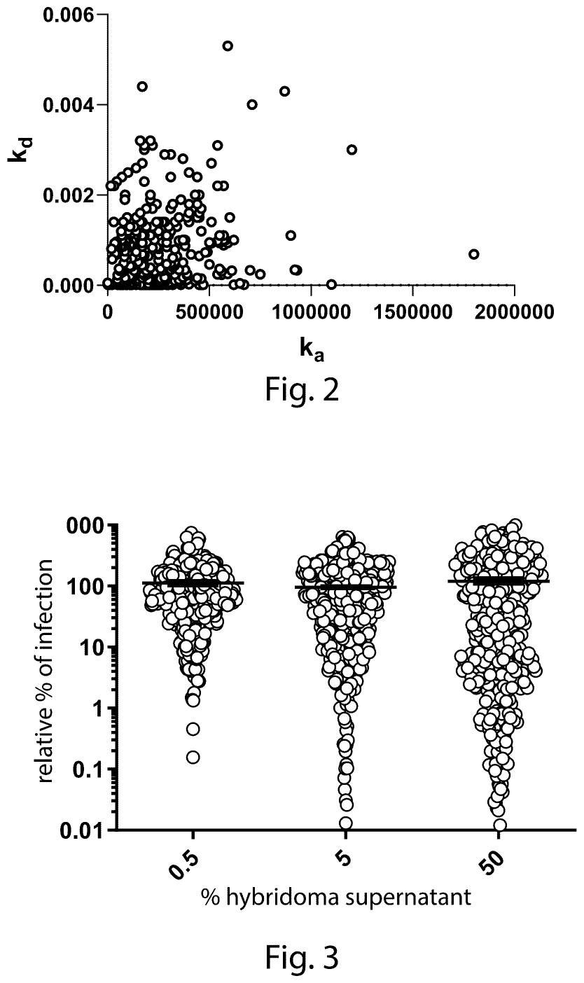

FIG. 2 shows the distribution of the association (Ka) and dissociation (Kd) constants; in particular, the graph shows the values of the association (ka) and dissociation (kd) constants obtained for the hybridomas analysed and determined by surface plasmon resonance using a Carterra LSA instrument;

FIG. 3 shows the pseudovirus assay; in particular, the graph shows the values, in relative percentage of infection, associated with the selected antibodies; all samples were analysed as a single replicate in three different dilutions (1:2, 1:20, 1:200);

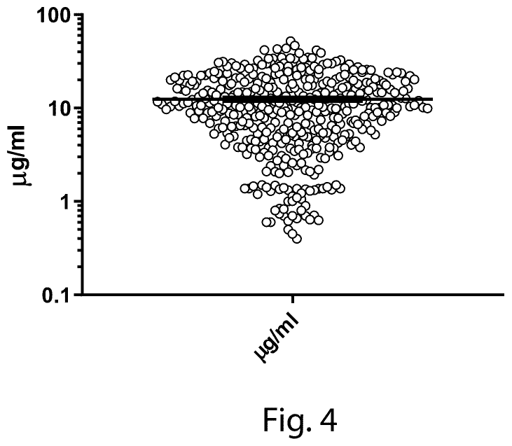

FIG. 4 shows the quantification of IgG; in particular, the graph shows the distribution of the quantification of total IgG in the samples analysed for the pseudovirus assay (the median is highlighted in black);

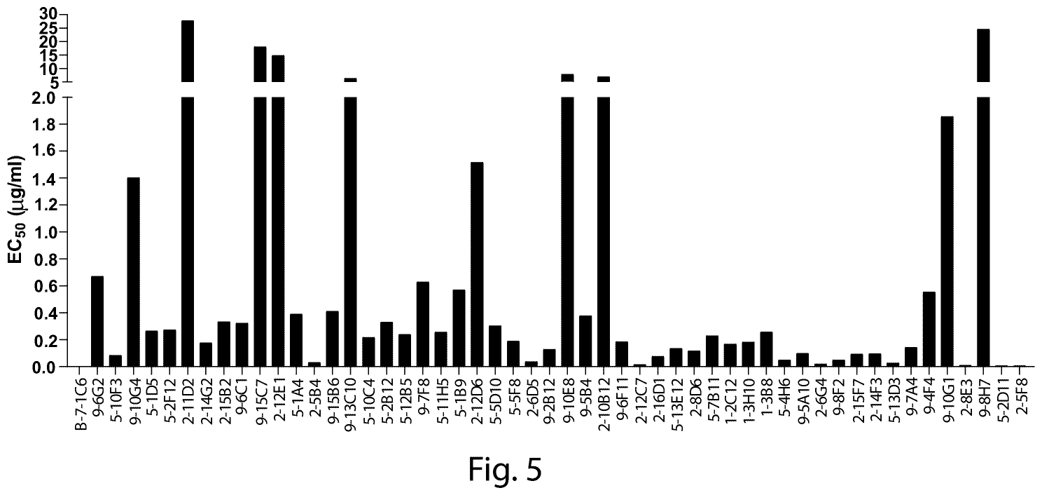

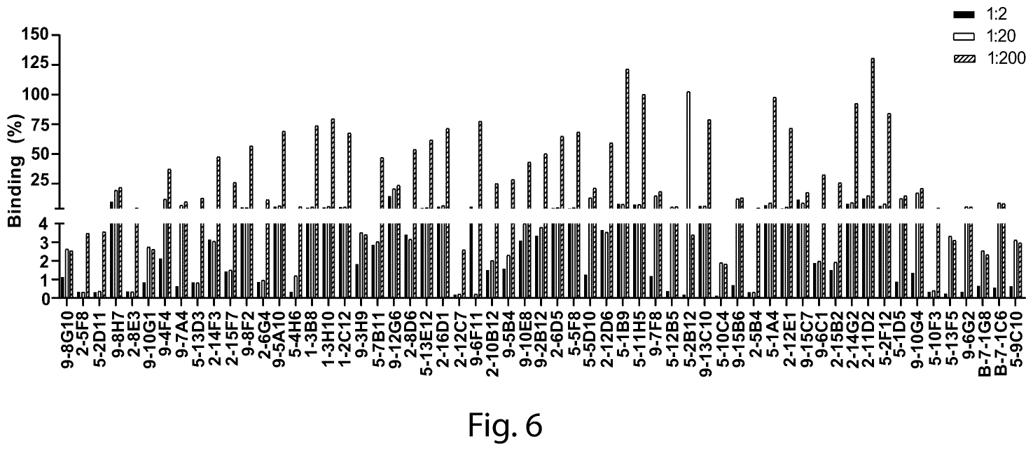

FIG. 5 shows the competitive ELISA results; in particular, the graph shows the EC50 values calculated for every antibody tested by means of competitive ELISA against human ACE2;

FIG. 6 shows the results of the competition assay by FACS; in particular, the graph shows the antibody binding percentages in the competition assay by FACS with human ACE2 in three scalar dilutions starting from 1:2; the capacity of the antibodies to compete with the binding between RBD-hFc and the ACE2 protein was assessed in Vero E6 cells;

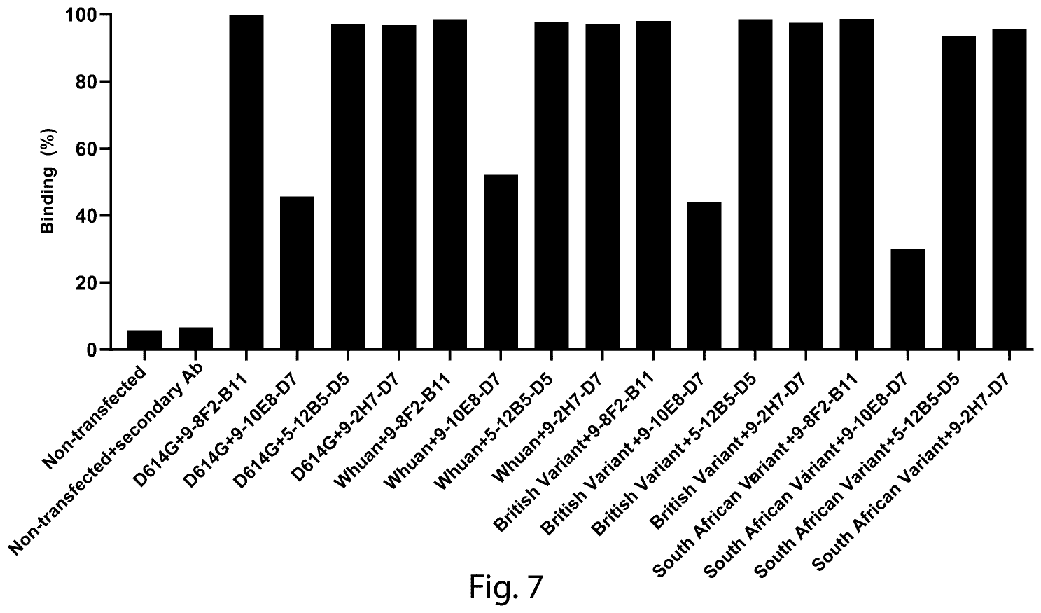

FIG. 7 shows the results of the FACS assay on RBD variants; in particular, the graph shows the purified antibody binding percentages in the FACS assay at a final concentration of 1 μg/ml; the HEK-293 cells were transfected with an empty vector (EV) as a negative control or pNebAd6 SARS-Cov-2 spike-FL or VOC;

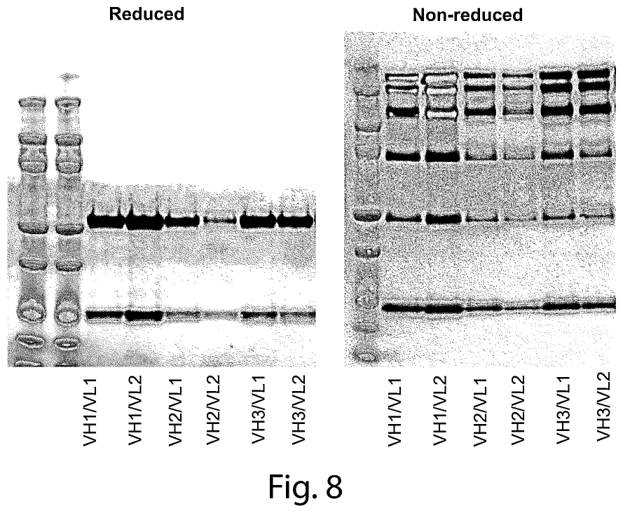

FIG. 8 shows the western blot of the chimeras of 9-8F2-B11; in particular, the figure shows the western blot of the culture medium deriving from transient transfections of ExpiCHO cells for the identification of VH/VL combinations for the chimeric antibody 9-8F2-B11; detection was performed with a secondary antibody specific for the human Fc portion;

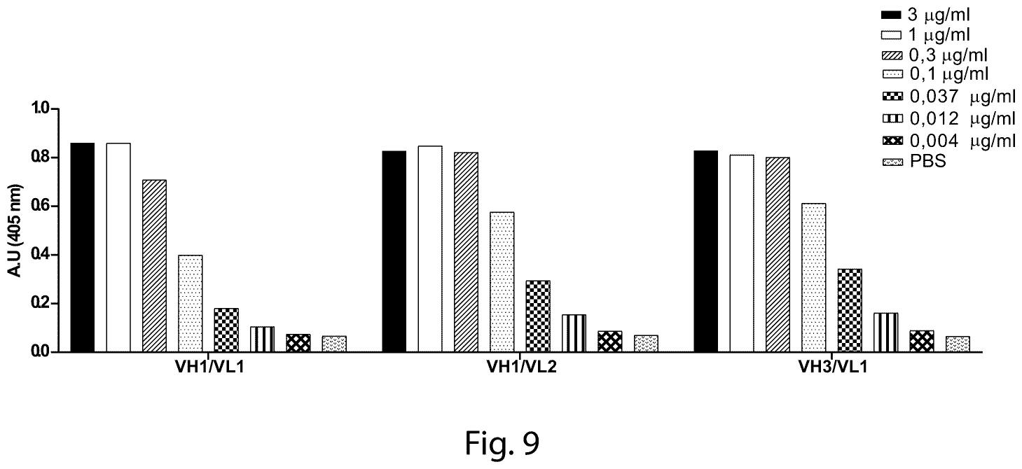

FIG. 9 shows the ELISA results for the chimeras of 9-8F2-B111; in particular, the histogram shows the absorbance values (405 nm) of the VH/VL combinations of the chimeric antibody 9-8F2-B11 tested for binding to RBD WT;

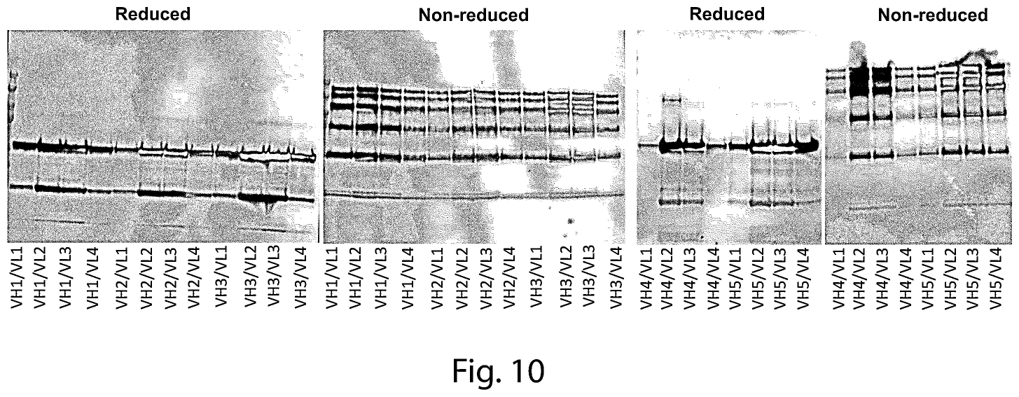

FIG. 10 shows the western blot of the humanised antibodies of 9-8F2-B11; in particular, the figure shows the western blot of the culture medium deriving from transient transfections of ExpiCHO cells for the identification of VH/VL combinations for the humanised antibody of 9-8F2-B111; detection was performed with a secondary antibody specific for the human Fc portion;

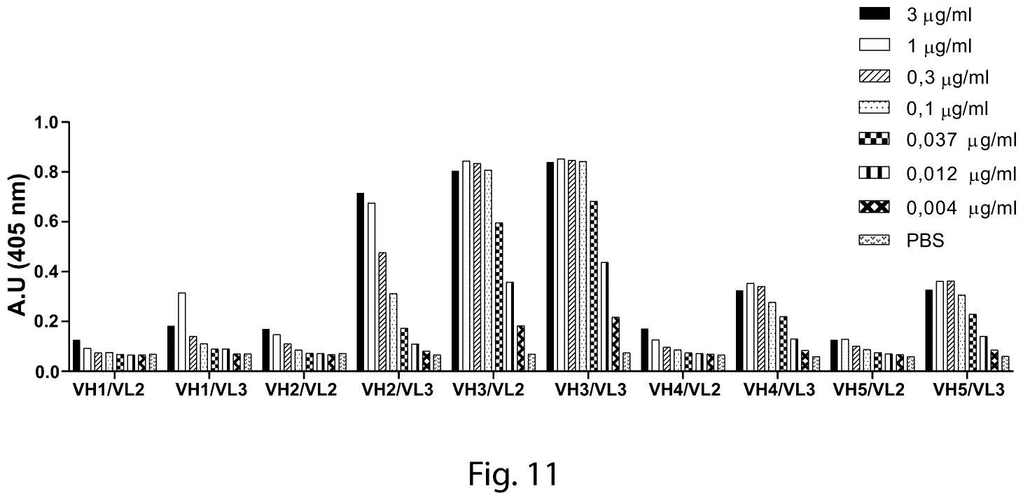

FIG. 11 shows the ELISA results for the humanised antibodies of 9-8F2-B11; in particular, the histogram shows the absorbance values (405 nm) of the VH/VL combinations of the humanised antibodies of 9-8F2-B111 tested for binding to RBD WT;

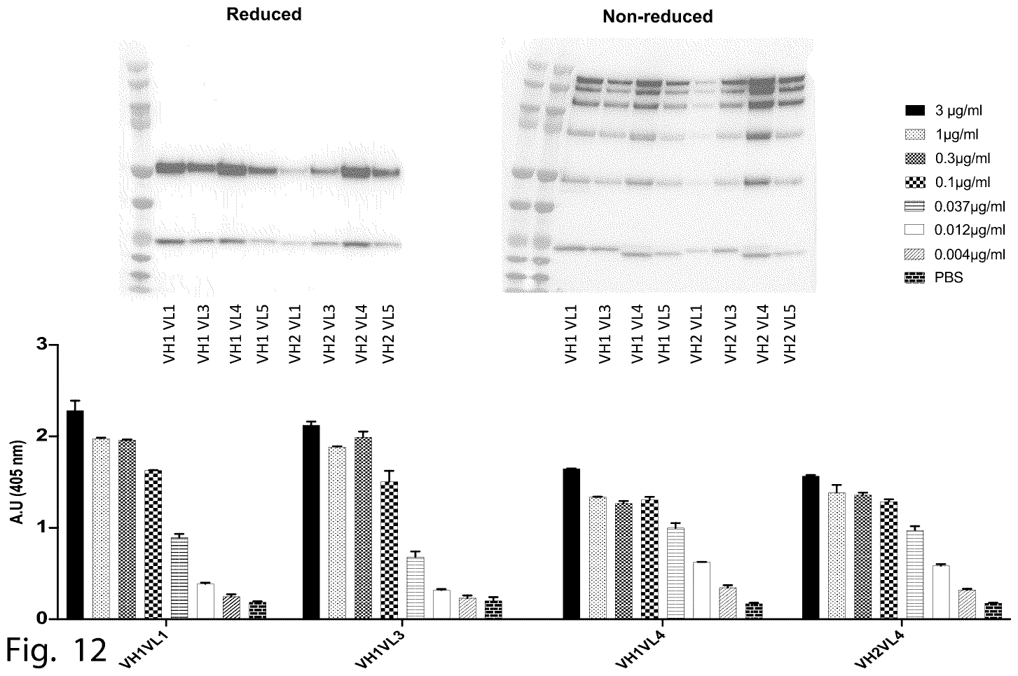

FIG. 12 shows the western blot of the chimeras of 9-7A4-C2 at the top; in particular, the figure shows the western blot of the culture medium deriving from transient transfections of ExpiCHO cells for the identification of VH/VL combinations for the chimeric antibody 9-7A4-C2; detection was performed with a secondary antibody specific for the human Fc portion; the ELISA results for the humanised antibodies of 9-7A4-C2 are shown at the bottom; in particular, the histogram shows the absorbance values (405 nm) of the VH/VL combinations of the antibody 9-7A4-C2 tested for binding to RBD WT

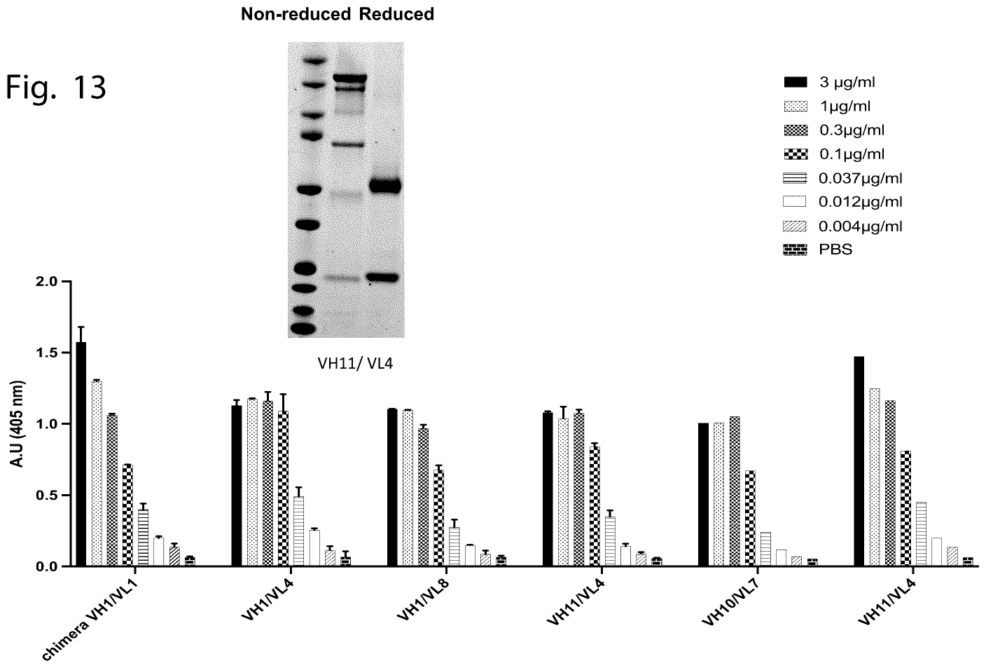

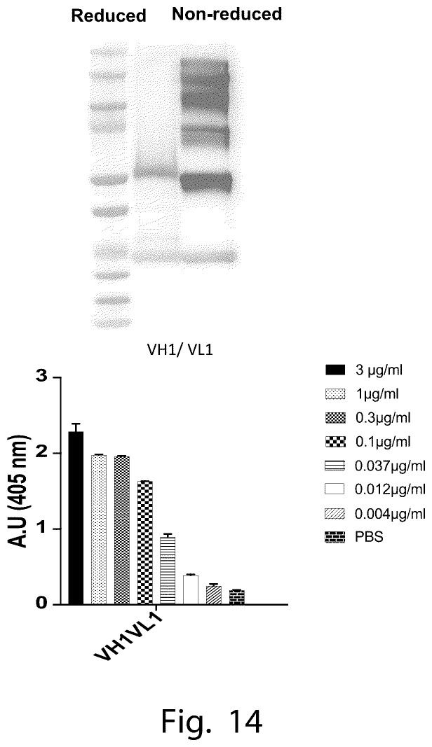

FIG. 13 shows the western blot of the humanised antibodies of 9-7A4-C2 at the top; in particular, the figure shows the western blot of the purified antibody deriving from transient transfections of ExpiCHO cells for the identification of VH/VL combinations for the chimeric antibody 9-7A4-C2; the ELISA results for the humanised antibodies of the 9-7A4-C2 are shown at the bottom; in particular, the histogram shows the absorbance values (405 nm) of the VH/VL combinations of the antibody 9-7A4-C2 tested for binding to RBD WT

FIG. 14 shows the western blot of the chimeras of 9-2H7-D7 at the top; in particular, the figure shows the western blot of the culture medium deriving from transient transfections of ExpiCHO cells for the identification of VH/VL combinations for the chimeric antibody 9-2H7-D7; detection was performed with a secondary antibody specific for the human Fc portion; the ELISA results for the humanised antibodies of 9-2H7-D7 are shown at the bottom; in particular, the histogram shows the absorbance values (405 nm) of the VH/VL combinations of the antibody 9-2H7-D7 tested for binding to RBD WT

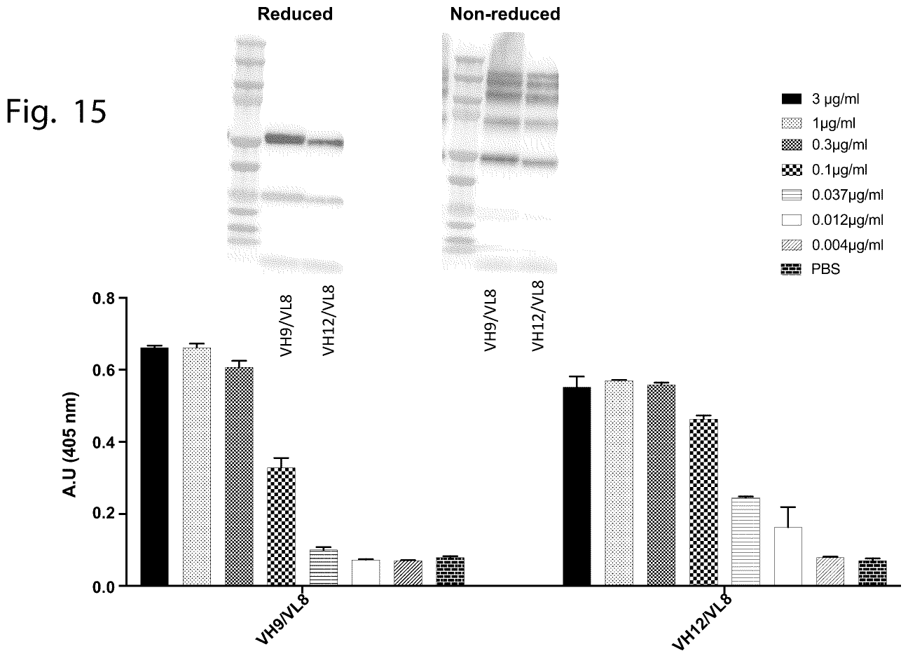

FIG. 15 shows the western blot of the humanised 9-2H7-D7 at the top; in particular, the figure shows the western blot of the purified antibody derived from transient transfections of ExpiCHO cells for the identification of VH/VL combinations for the chimeric antibody 9-2H7-D7; the ELISA results for the humanised antibodies of 9-2H7-D7 are shown at the bottom; in particular, the histogram shows the absorbance values (405 nm) of the VH/VL combinations of the antibody 9-2H7-D7 tested for binding to RBD WT

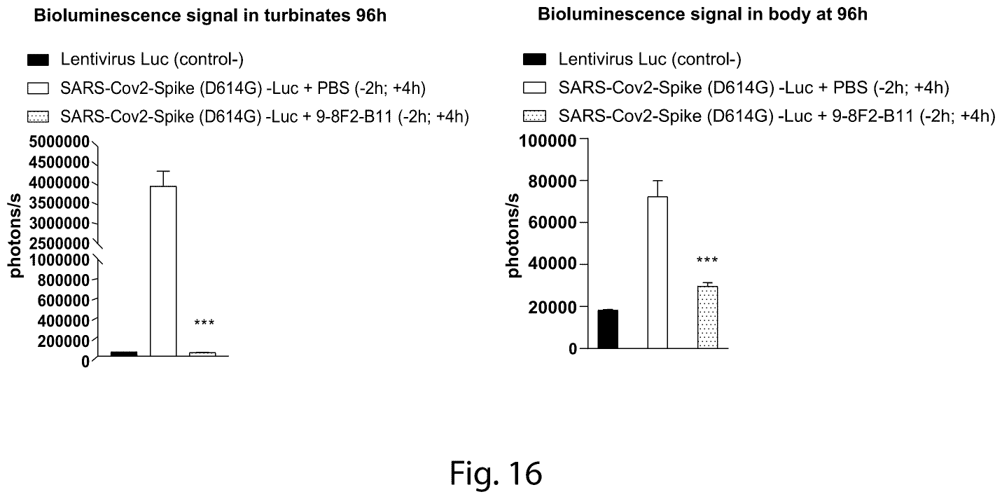

FIG. 16 shows the results of the in vivo pseudovirus assay; in particular, the graphs show the mean values±SD (photon/s) of the bioluminescence detected in the K18-hACE2 mice both in the turbinates (graph on the left) and in the whole body of the animal (graph on the right) at 96 h after transfection;

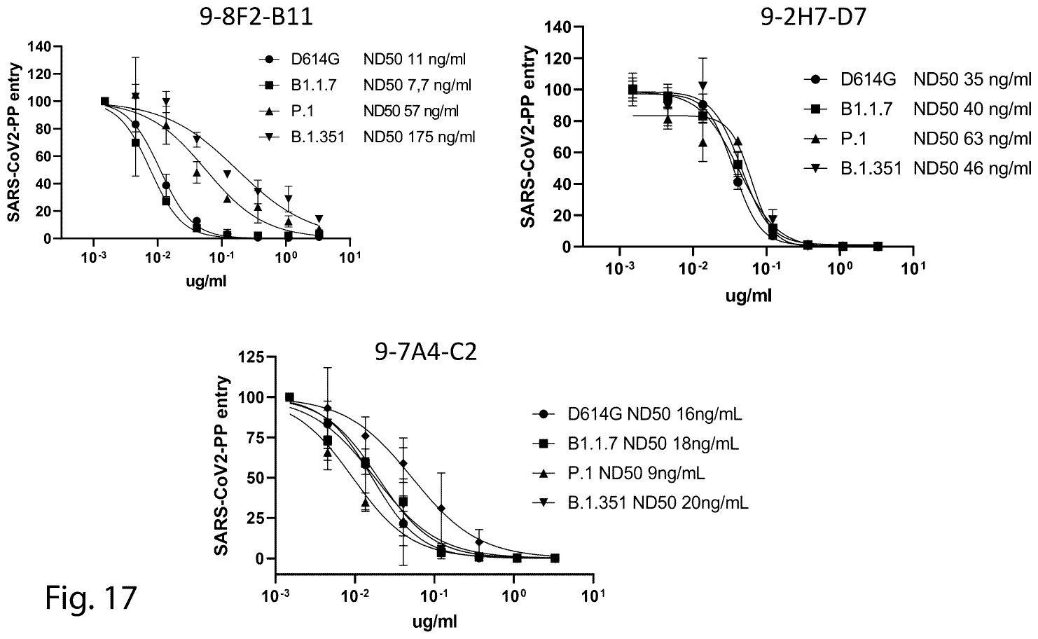

FIG. 17 shows the neutralisation curve with respective IC50s (ng/ml) calculated respectively for the purified mouse antibodies 9-8F2-B11, 92H7-D7 and 9-7A4-C2, against the pseudovirus variants D614G, B1.1.7 (British), P.1 (Brazilian), B.1.351 (African).

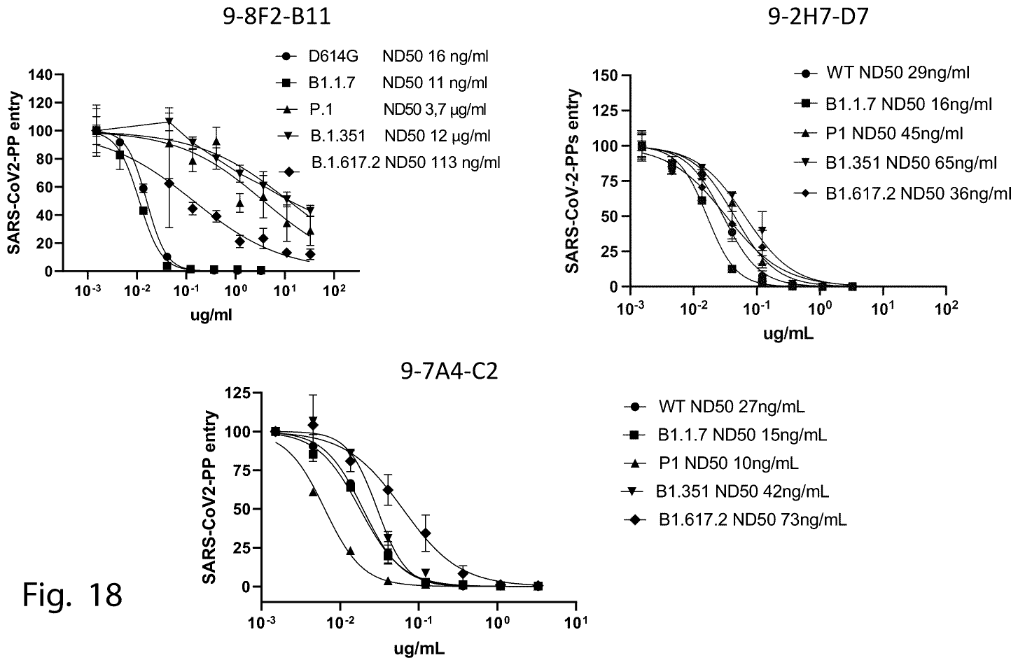

FIG. 18 shows the neutralisation curve with respective IC50s (ng/ml) calculated respectively for the purified humanised antibodies 9-8F2-B11, 92H7-D7 and 9-7A4-C2, against the pseudovirus variants D614G, B1.1.7 (British), P.1 (Brazilian), B.1.351 (African) and delta.

### EXAMPLE 1. IDENTIFICATION AND EVALUATION OF THE EFFECTIVENESS OF THE ANTIBODIES ACCORDING TO THE PRESENT INVENTION

### Materials and Methods

### Measurement of Antibody Titre Against RBD Protein.

In order to identify the animals with the highest antibody titre to be used for the generation of hybridomas, a titration of the IgG antibodies against the RBD portion of the S protein was performed by means of the ELISA technique on day 27 after the first treatment (FIG. 1).

The ELISA plates are functionalised by coating with the RBD-6×His protein at a concentration of 1 μg/ml and incubated for about 18 hours at 4° C. Subsequently, the plates are blocked with 3% BSA/0.05% Tween-20/PBS for 1 hour at room temperature and then the excess solution is eliminated by overturning. The sera of the immunised mice are then added, starting from a dilution of 1/400 and serially diluting 1:3 until reaching a dilution of 1/874800, in duplicate, and the plates are incubated for 2 hours at room temperature. After double washing with 0.05% Tween-20/PBS, secondary anti-mouse IgG, Human Ads-alkaline phosphatase conjugated antibody is added, and the plates are incubated for 1 hour at room temperature. After double washing with 0.05% Tween-20/PBS, the binding of the secondary antibody is detected by adding the substrate for alkaline phosphatase and measuring the absorbance at 405 nm by means of an ELISA reader after 1 hour of incubation.

### Quantification of Antibody Concentration by Means of Bio-Layer Interferometry.

A rapid quantification of the monoclonal antibody present in the culture medium of the hybridoma was performed by bio-layer interferometry (BLI) using the Octet RED96 system (FORTEBIO). The analyses were performed using anti-mouse Fc biosensors (Anti-mouse IgG Fc Capture Biosensors, cat. No. 18-5088, FORTEBIO) at a temperature of 30° C. with shaking at 1000 rpm in 96-well plates (96-well microplates, black, 655209, Greiner Bio-One). The samples were diluted 1:10 in a diluent buffer (sample diluent 18-1104, FORTEBIO) to reduce the interference of the medium and a total volume of 200 μl of solution was loaded per well. At this point, the biosensor tips were immersed for 10 minutes in the running buffer prepared by diluting the medium of the hybridomas 1:10 in the diluent buffer. A calibration curve was prepared using a purified reference antibody diluted in running buffer (concentration range: 0.195-25 μg/mL). The quantifications were performed by taking into consideration the initial values (from 0 to 180 s) of the binding responses. The sample concentration was calculated from the standard curve using Octet Software V11.1. To evaluate the quality of the calibration curve, the residual (%) of each calibrator was estimated as lower than 18% and the r2 of the curve used to determine the binding rate was selected as >0.98. (FIG. 4)

### Production of RBD-Fc and RBD-6×his WT Proteins and Mutants

The RBD-Fc and RBD-6×His proteins were produced by transient transfection of Expi293F high density cells (Expi293™ Expression System Kit, A14635) with ExpiFectamine 293 cationic lipid transfection reagent (Thermo Fisher) according to the manufacturer's instructions. The supernatant containing the proteins was collected after a week and subjected to clarification by centrifugation and filtration for the subsequent purification steps. The protein RBD-Fc was purified by means of the AktaPure affinity chromatography system with a protein A column (TOYOSCREEN AF-RPROTEIN A HC-650F; Tosoh Bioscience). Briefly, the column was equilibrated with a binding buffer (Phosphate Buffer 0.1M Ph8) and loaded with the supernatant diluted 1:1 with the same buffer. After washing of the column, the protein was recovered by acid elution in citrate buffer 0.1M pH3, neutralised in Tris-HCl pH9 and subjected to dialysis in PBS1× with a slide-A-lyzer (Thermo Fisher) according to the directions in the product datasheet.

The RBD-6×HIS protein and the mutants were purified by immobilised metal affinity chromatography of His Tag residues using the AktaPure system with HisPur™ Ni-NTA Chromatography Cartridges (Thermo Fisher) according to the manufacturer's instructions. Briefly, the column was equilibrated in PBS1×/Imidazole 5 mM and loaded with the supernatant diluted 1:1 with the same buffer. After washing, the protein was eluted with PBS1×/Imidazole 0.3M, pH 7.4 and dialysed in PBS1× with a slide-A-lyzer (Thermo Fisher) according to the directions in the product datasheet. After being recovered by dialysis, the RBD-Fc and RBD-6×His proteins were quantified by spectrophotometry with absorbance at 280 nm.

The purity of the proteins was evaluated by SDS-PAGE and western blot analysis, conducted under both reduced and non-reduced conditions and with standard methods.

### Pseudovirus Neutralisation Assay

For the pseudovirus assay, a set of antibodies selected based on their affinity for SARS-CoV-2 recombinant RBD were analysed (values comprised between 10 pM and 100 pM, several nM values). The antibodies were analysed as a single replicate in three different dilutions (1:2, 1:20, 1:200) (FIG. 3) or scalar concentrations in order to calculate the EC50 thereof using the vesicular stomatitis pseudovirus (VSV)(Fluc_eGFP)-CoV2-PP with Fluc as the reporter. For the three antibodies selected as preferred candidates—9-8F2-B11, 9-2H7-D7 and 9-7A4-C2—an initial concentration of 3.3 μg/ml of the purified antibody was used for scalar dilutions to calculate the EC50 using the vesicular stomatitis pseudovirus (VSV)(Fluc_eGFP)-CoV2-PP with Fluc as the reporter. One day before the assay, 10,000 Caco-2 cells (ECACC catalogue no.09042001) were seeded in 96-well plates (180 μl/96 w); the day after cell seeding, the hybridoma samples were diluted in PBS and incubated with pseudoparticles (1:1 mixture) for 1 hour at 37 degrees and the Fluc reporter was measured 16-20 hours after transduction. The plates were centrifuged for 1 minute at a speed of 270 g; the supernatant was removed, and then followed washing with PBS, 100 μl per well. After a further centrifugation and removal of the supernatant, 40 μl of lysis buffer were added for the luciferase assay, with subsequent incubation of 5 minutes. 30 μl of the volume were subsequently transferred to a blank plate; 50 μl of Luciferase Reagent were added to this volume and total luminescence was measured. The EC50 values were normalised for the concentration of antibodies in the supernatant (FIG. 3, Table 3). For the purified antibodies 9-8F2-B11, 9-2H7-D7, 9-7A4-C2, the procedure carried out provides for the same steps as described above (FIGS. 17 and 18).

### ELISA Competition Assay

In order to evaluate the capacity of the antibodies to compete with binding between ACE2 and RBD, a second assay based on the ELISA technique was used. In accordance with the SPR data, the majority of the antibodies proved to be competitive, and the results are summarised in FIG. 5. The plates were coated with 50 ul per well of the RBD-6His protein diluted in PBS to a final concentration of 1.42 μg/ml. The coating step was carried out overnight at a temperature of 4 degrees. The plates were subsequently washed three times with PBS/T and then blocked with 100 ul of blocking solution (3% BSA) for a period of one hour at room temperature. Then followed the competition phase, in which the hybridoma supernatant and ACE2-hFc were incubated overnight at 4 degrees, at a concentration of 2 μg/ml, in 1% BSA. The plates were washed three times with PBS/T and were subsequently incubated with anti-Human AP secondary antibody (Jackson 209-055-098) at a final dilution of 1:5000. The plates were subsequently developed with a development reagent at room temperature for 1 h; then followed a reading at a wavelength of 405 nm (FIG. 5).

### FACS Competition Assay

The capacity of the antibodies to compete with binding between RBD-hFc and the ACE2 protein expressed by Vero E6 cells (VERO C1008C1008 [Vero 76, clone E6, Vero E6] (ATCC® CRL-1586™) was further evaluated by flow cytometry and the results are summarised in FIG. 6, where one may observe an almost complete inhibition of the binding of RBD-hFc to the ACE2 protein when the supernatants are used at the lowest dilution (1:2). Briefly, the monoclonal antibodies were diluted to different dilutions (1:2, 1:20, 1:200) in RBD-hFc (0.45 μg/ml) in FC buffer and incubated for 1 hour at 4° C. 200,000 cells/well (96-well plate) were then stained with RBD-hFc or preincubated serum/RBD-hFc for 25 minutes at 4° C. The cells were then washed with FC buffer and stained with anti-human IgG Alexa Fluor 488-conjugated secondary antibodies for 25 minutes at 4° C. Finally, the cells were washed in FC buffer and resuspended in FC buffer before being run on a CytoFlex flow cytometer (Beckman Coulter). The analysis was performed using CytExpert software (Beckman Coulter).

### Production and Purification of Mouse Antibodies

The clones of interest, after confirmation of positivity, were gradually adapted after thawing from a medium containing 10% FBS to a CD Hybrid medium (Gibco), cultured and amplified under static conditions until reaching a final volume of about 400 ml. The supernatant containing the monoclonal antibodies produced by the hybridomas was collected and subjected to clarification by centrifugation and filtration for the subsequent purification steps. The monoclonal antibodies were purified by means of the AktaPure affinity chromatography system with a protein A column (TOYOSCREEN AF-RPROTEIN A HC-650F; Tosoh Bioscience). Briefly, the column was equilibrated with binding buffer (Phosphate Buffer 0.1M Ph8) and loaded with the supernatant diluted 1:1 with the same buffer. After washing of the column, the protein was recovered by acid elution in 0.1M buffer citrate pH3, neutralised in Tris-HCl pH9 and subjected to dialysis in PBS1× with a slide-A-lyzer (Thermo Fisher) according to the directions in the product datasheet.

### Results

### Genetic Vaccination

To increase the probability of obtaining anti-RBD antibodies that were neutralising from a functional viewpoint, a genetic vaccination approach based on electroporation in skeletal muscle was adopted (32). This technology allows the antigen of interest to be appropriately modified using molecular engineering techniques which enable the endogenous expression thereof in muscle and in cells showing the antigen. Wild-type spike cDNA was used for immunisation; a plasmid vector containing a modified version with optimised codons which expresses the complete form of the SARS-CoV-2 spike protein was injected into BALB/C mice (Cat: BALB/cOlaHsd—Envigo). It was hypothesised that immunisation with this variant might increase the probability of obtaining neutralising antibodies, as it was exposed on the cell membrane.

The immunisation protocol consisted in 2 injections of 50 μg of pTK1A-Covid-FL into the animal's quadriceps, alternated with 2 intraperitoneal injections of 10 μg of RBD-hFc protein, spaced apart from one another by 2 weeks. The mice were thus immunised on days 0 and 14 by genetic immunisation using the pTK1A-Covid-FL plasmid, whereas they were immunised with the purified RBD-hFc protein on days 7 and 21 after the start of treatment.

The pTK1A-Covid-FL plasmid comprised the promoter and intron A of human cytomegalovirus (CMV), a polylinker site for the cloning and bovine growth hormone (bGH) as the polyA signal for the termination of transcription. Furthermore, the pTK1A-Covid-FL plasmid comprised the following optimised sequence coding for the full-length Spike protein:

(SEQ ID NO: 35)
atgtttgtcttcctggtcctgctgcccctggtctcctctcagtgc
gtgaacctgactactagaactcagctgcctcccgcttacaccaat
tccttcacccggggcgtgtactatcctgacaaggtgtttagaagc
tccgtgctgcactctacacaggatctgtttctgccattctttagc
aacgtgacctggttccacgccatccacgtgagcggcaccaatggc
acaaagcggttcgacaatcccgtgctgccttttaacgatggcgtg
tacttcgcctctaccgagaagagcaacatcatcagaggctggatc
tttggcaccacactggactccaagacacagtctctgctgatcgtg
aacaatgccaccaacgtggtcatcaaggtgtgcgagttccagttt
tgtaatgatcccttcctgggcgtgtactatcacaagaacaataag
agctggatggagtccgagtttagagtgtattctagcgccaacaat
tgcacatttgagtacgtgtcccagcctttcctgatggacctggag
ggcaagcagggcaatttcaagaacctgagggagttcgtgtttaag
aatatcgatggctacttcaaaatctactctaagcacacccccatc
aacctggtgcgcgacctgcctcagggcttcagcgccctggagcca
ctggtggatctgcctatcggcatcaacatcacccggtttcagaca
ctgctggccctgcacagaagctacctgacacccggcgactcctct
agcggatggaccgcaggagcagcagcctactatgtgggctatctg
cagcctaggaccttcctgctgaagtacaacgagaatggcaccatc
acagacgccgtggattgcgccctggatcctctgagcgagacaaag
tgtacactgaagtcctttaccgtggagaagggcatctatcagaca
tccaatttcagggtgcagccaaccgagtctatcgtgcgctttcct
aatatcacaaacctgtgcccatttggcgaggtgttcaacgcaacc
aggttcgcaagcgtgtacgcatggaataggaagcgcatctctaac
tgcgtggccgactatagcgtgctgtacaactccgcctctttcagc
acctttaagtgctatggcgtgtcccccacaaagctgaatgacctg
tgctttaccaacgtgtacgccgattctttcgtgatcaggggcgac
gaggtgcgccagatcgcacctggacagacaggcaagatcgccgac
tacaattataagctgccagacgatttcaccggctgcgtgatcgcc
tggaacagcaacaatctggattccaaagtgggcggcaactacaat
tatctgtaccggctgtttagaaagagcaatctgaagcccttcgag
agggacatctctacagaaatctaccaggccggcagcaccccttgc
aatggcgtggagggctttaactgttatttcccactgcagtcctac
ggcttccagcccacaaacggcgtgggctatcagccttaccgcgtg
gtggtgctgagctttgagctgctgcacgcaccagcaacagtgtgc
ggacccaagaagtccaccaatctggtgaagaacaagtgcgtgaac
ttcaacttcaacggcctgaccggaacaggcgtgctgaccgagtcc
aacaagaagttcctgccatttcagcagttcggcagggacatcgca
gataccacagacgccgtgcgcgacccacagaccctggagatcctg
gatatcacaccctgctctttcggcggcgtgagcgtgatcacacca
ggaaccaatacaagcaaccaggtggccgtgctgtatcaggacgtg
aattgtaccgaggtgcctgtggccatccacgccgatcagctgacc
ccaacatggcgggtgtacagcaccggctccaacgtgttccagaca
agagcaggatgtctgatcggagcagagcacgtgaacaattcctat
gagtgcgacatcccaatcggcgccggcatctgtgcctcttaccag
acccagacaaactctccaaggagagcacggagcgtggcatcccag
tctatcatcgcctataccatgtccctgggcgccgagaattctgtg
gcctactctaacaatagcatcgccatcccaaccaacttcacaatc
tctgtgaccacagagatcctgcccgtgtccatgaccaagacatct
gtggactgcacaatgtatatctgtggcgattctaccgagtgcagc
aacctgctgctgcagtacggcagcttttgtacccagctgaataga
gccctgacaggcatcgccgtggagcaggataagaacacacaggag
gtgttcgcccaggtgaagcaaatctacaagaccccccctatcaag
gactttggcggcttcaatttttcccagatcctgcctgatccatcc
aagccttctaagcggagctttatcgaggacctgctgttcaacaag
gtgaccctggccgatgccggcttcatcaagcagtatggcgattgc
ctgggcgacatcgcagcacgggacctgatctgtgcccagaagttt
aatggcctgaccgtgctgccacccctgctgacagatgagatgatc
gcacagtacacaagcgccctgctggcaggaaccatcacatccgga
tggaccttcggcgcaggagccgccctgcagatcccctttgccatg
cagatggcctataggttcaacggcatcggcgtgacccagaatgtg
ctgtacgagaaccagaagctgatcgccaatcagtttaactccgcc
atcggcaagatccaggacagcctgtcctctacagcctccgccctg
ggcaagctgcaggatgtggtgaatcagaacgcccaggccctgaat
accctggtgaagcagctgagctccaacttcggcgccatctctagc
gtgctgaatgatatcctgagccggctggacaaggtggaggcagag
gtgcagatcgaccggctgatcacaggcagactgcagtctctgcag
acctatgtgacacagcagctgatcagggcagcagagatcagggca
agcgccaatctggcagcaaccaagatgtccgagtgcgtgctgggc
cagtctaagagagtggacttttgtggcaagggctatcacctgatg
tccttccctcagtctgccccacacggcgtggtgtttctgcacgtg
acctacgtgcccgcccaggagaagaacttcaccacagcccctgcc
atctgccacgatggcaaggcccactttccaagggagggcgtgttc
gtgtccaacggcacccactggtttgtgacacagcgcaatttctac
gagccccagatcatcaccacagacaataccttcgtgagcggcaac
tgtgacgtggtcatcggcatcgtgaacaataccgtgtatgatcca
ctgcagcccgagctggacagctttaaggaggagctggataagtac
ttcaagaatcacacctcccctgacgtggatctgggcgacatcagc
ggcatcaatgcctccgtggtgaacatccagaaggagatcgaccgc
ctgaacgaggtggccaagaatctgaacgagagcctgatcgatctg
caggagctgggcaagtatgagcagtacatcaagtggccatggtac
atctggctgggcttcatcgccggcctgatcgccatcgtgatggtg
accatcatgctgtgctgtatgacatcctgctgttcttgcctgaag
ggctgctgtagctgcggctcctgttgtaagtttgatgaagacgat
tccgagcctgtcctgaagggcgtgaagctgcactatacctctaga
taatga,
encoding for the following amino acid sequence:
(SEQ ID NO: 39)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKF
NGLTVLPPLLTDEMIAQYTSALLAGTITSGWTFGAGAALQIPFAM
QMAYRFNGIGVTQNVLYENQKLIANQFNSAIGKIQDSLSSTASAL
GKLQDVVNQNAQALNTLVKQLSSNFGAISSVLNDILSRLDKVEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFDEDD
SEPVLKGVKLHYTSR,

wherein the portion of the sequence in bold indicates the RBD region of the Spike protein.

The DNA was formulated in phosphate-buffered saline (PBS) at a concentration of 1 mg/ml. DNA-EP was performed with a Cliniporator electroporator and flat electrodes (IGEA, Carpi, Italy) under the following electrical conditions in the electro-gene-transfer (EGT) mode: eight 20 msec pulses at 110V, 8 Hz, 120 msec pause between pulses.

The RBD-hFc protein administered to the mice had the following sequence:

(SEQ ID NO: 40)
METDTLLLWVLLLWVPGSTGRVQPTESIVRFPNITNLCPFGEVFN
ATRFASVYAWNRKRISNCVADYSVLYNSASFSTFKCYGVSPTKLN
DLCFTNVYADSFVIRGDEVRQIAPGQTGKIADYNYKLPDDFTGCV
IAWNSNNLDSKVGGNYNYLYRLFRKSNLKPFERDISTEIYQAGST
PCNGVEGFNCYFPLQSYGFQPTNGVGYQPYRVVVLSFELLHAPAT
VCGPKKSTNLVKNKCVNFVDKTHTCPPCPAPELLGGPSVFLFPPK
PKDTLMISRTPEVTCVVVDVSHEDPEVKFNWYVDGVEVHNAKTKP
REEQYNSTYRVVSVLTVLHQDWLNGKEYKCKVSNKALPAPIEKTI
SKAKGQPREPQVYTLPPSREEMTKNQVSLTCLVKGFYPSDIAVEW
ESNGQPENNYKTTPPVLDSDGSFFLYSKLTVDKSRWQQGNVFSCS
VMHEALHNHYTQKSLSLSPGK,
encoded by the following nucleotide sequence
(SEQ ID NO: 41)
atggagacagacacactcctgctatgggtactgctgctctgggtt
ccaggatccacaggaagagtccaaccaacagaatctattgttaga
tttcctaatattacaaacttgtgcccttttggtgaagtttttaac
gccaccagatttgcatctgtttatgcttggaacaggaagagaatc
agcaactgtgttgctgattattctgtcctatataattccgcatca
ttttccacttttaagtgttatggagtgtctcctactaaattaaat
gatctctgctttactaatgtctatgcagattcatttgtaattaga
ggtgatgaagtcagacaaatcgctccagggcaaactggaaagatt
gctgattataattataaattaccagatgattttacaggctgcgtt
atagcttggaattctaacaatcttgattctaaggttggtggtaat
tataattacctgtatagattgtttaggaagtctaatctcaaacct
tttgagagagatatttcaactgaaatctatcaggccggtagcaca
ccttgtaatggtgttgaaggttttaattgttactttcctttacaa
tcatatggtttccaacccactaatggtgttggttaccaaccatac
agagtagtagtactttcttttgaacttctacatgcaccagcaact
gtttgtggacctaaaaagtctactaatttggttaaaaacaaatgt
gtcaatttcgtcgacaaaactcacacatgcccaccgtgcccagca
cctgaactcctggggggaccgtcagtcttcctcttccccccaaaa
cccaaggacaccctcatgatctcccggacccctgaggtcacatgc
gtggtggtggacgtgagccacgaagaccctgaggtcaagttcaac
tggtacgtggacggcgtggaggtgcataatgccaagacaaagccg
cgggaggagcagtacaacagcacgtaccgtgtggtcagcgtcctc
accgtcctgcaccaggactggctgaatggcaaggagtacaagtgc
aaggtctccaacaaagccctcccagcccccatcgagaaaaccatc
tccaaagccaaagggcagccccgagaaccacaggtgtacaccctg
cccccatcccgggaggagatgaccaagaaccaggtcagcctgacc
tgcctggtcaaaggcttctatcccagcgacatcgccgtggagtgg
gagagcaatgggcagccggagaacaactacaagaccacgcctccc
gtgctggactccgacggctccttcttcctctacagcaagctcacc
gtggacaagagcaggtggcagcaggggaacgtcttctcatgctcc
gtgatgcatgaggctctgcacaaccactacacgcagaagagcctc
tccctgtctccgggtaaa.

The portion of the sequence highlighted above in bold, namely RVQPTESIVRFPNITNLCPFGEVFNATRFASVYAWNRKRISNCVADYSVL YNSASFSTFKCYGVSPTKLNDLCFTNVYADSFVIRGDEVRQIAPGQTGKI ADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFE LLHAPATVCGPKKSTNLVKNKCVNF (SEQ ID NO:36) represents RBD, the underlined portion of the sequence, namely METDTLLLWVLLLWVPGSTG (SEQ ID NO:37), represents the signal peptide of IgK chain, whilst the portion of the sequence in italics, namely VDKTHTCPPCPAPELLGGPSVFLFPPKPKDTLMISRTPEVTCVVVDVSHE DPEVKFNWYVDGVEVHNAKTKPREEQYNSTYRVVSVLTVLHQDWLNGK EYKCKVSNKALPAPIEKTISKAKGQPREPQVYTLPPSREEMTKNQVSLTC LVKGFYPSDIAVEWESNGQPENNYKTTPPVLDSDGSFFLYSKLTVDKSR WQQGNVFSCSVMHEALHNHYTQKSLSLSPGK (SEQ ID NO:38), represents the Fc domain.

The RBD-hFc protein was formulated in the PBS/Sigma Adjuvant System (1:1). All the immunised mice (14 animals) responded to the vaccination and their sera showed a significant activity of binding to RBD-6×His when assayed by means of the ELISA technique (FIG. 1).

Two weeks after the fourth immunisation, the mice with the highest antibody titre were sacrificed and spleens and lymph nodes removed. A standard protocol of fusion with mouse myeloma cells, as described in Harlow et al., “Antibodies: a laboratory manual”, was carried out; then 10000 (11819) hybridoma clones were isolated for serial dilution and the supernatants thereof were tested again by means of the ELISA technique for the capacity to bind RBD in vitro.

Among the hybridomas analysed, 1750 hybridomas, listed in Table 1, were selected for their capacity to bind the RBD as determined by ELISA. In particular, Table 1 shows the values related to the ability of the antibodies contained in the supernatant of each hybridoma to bind to the RBD fragment as determined by ELISA (A405 nm) and SPR(K_{D}).

TABLE 1

| Hybridoma | A(405 | | Hybridoma | A(405 | |
|---|---|---|---|---|---|
| Name | nm) | K_{D}(M) | Name | nm) | K_{D}(M) |
| 1-2C12 | 3.416 | 4.78E−10 | 2-2G5 | 1.731 | 1.77E−08 |
| 1-2G7 | 3.366 | 3.25E−10 | 2-2G7 | 0.737 | 5.51E−11 |
| 1-3B8 | 3.478 | 3.32E−10 | 2-2G9 | 2.127 | 6.39E−11 |
| 1-3H10 | 3.431 | 3.58E−10 | 2-2H7 | 2.457 | 1.98E−09 |
| 1-5D10 | 3.445 | 3.67E−10 | 2-3A5 | 1.288 | 3.36E−08 |
| 1-5F12 | 3.057 | 6.11E−09 | 2-3B1 | 1.761 | 2.25E−10 |
| 1-5G6 | 3.358 | 1.57E−09 | 2-3B2 | 0.782 | 7.53E−11 |
| 2-1A1 | 2.147 | 8.12E−11 | 2-3B5 | 2.132 | 3.88E−10 |
| 2-1 A2 | 2.310 | 3.86E−11 | 2-3B6 | 2.148 | 5.04E−08 |
| 2-1A10 | 2.004 | 1.16E−08 | 2-3B9 | 1.846 | 8.77E−09 |
| 2-1D3 | 1.459 | 7.93E−11 | 2-3C11 | 1.148 | 2.71E−09 |
| 2-1D8 | 2.142 | 4.33E−09 | 2-3D4 | 3.568 | 1.57E−10 |
| 2-1E3 | 1.924 | 1.08E−08 | 2-3D6 | 3.563 | 1.45E−08 |
| 2-1E6 | 2.153 | 2.85E−09 | 2-3E10 | 1.445 | 2.92E−09 |
| 2-1E12 | 1.482 | 1.29E−09 | 2-3G2 | 3.395 | 5.57E−11 |
| 2-1F2 | 1.876 | 7.72E−09 | 2-3G5 | 3.583 | 1.43E−09 |
| 2-1F10 | 1.284 | 1.99E−09 | 2-3G10 | 1.106 | 8.93E−11 |
| 2-1F11 | 2.780 | 4.12E−10 | 2-3H6 | 0.813 | n/a |
| 2-1G1 | 1.351 | 1.10E−08 | 2-4A1 | 3.538 | 1.21E−09 |
| 2-1G5 | 1.354 | 2.71E−09 | 2-4A3 | 3.149 | 2.39E−09 |
| 2-1G9 | 2.106 | 1.18E−09 | 2-4A9 | 2.307 | 3.65E−09 |
| 2-1H1 | 1.781 | 5.47E−10 | 2-4A10 | 2.128 | 1.19E−10 |
| 2-1H3 | 1.408 | 1.04E−08 | 2-4B1 | 1.983 | 3.54E−11 |
| 2-1H8 | 1.867 | 8.03E−09 | 2-4B5 | 1.534 | 7.92E−11 |
| 2-2A3 | 1.625 | 1.12E−10 | 2-4C11 | 3.267 | 2.53E−09 |
| 2-2B2 | 1.679 | 2.63E−09 | 2-4D1 | 3.253 | 4.06E−09 |
| 2-2C4 | 1.337 | 5.73E−09 | 2-4D2 | 1.277 | 2.91E−09 |
| 2-2C5 | 1.636 | n/a | 2-4D7 | 2.548 | 6.49E−10 |
| 2-2D4 | 2.782 | n/a | 2-4E4 | 1.493 | 3.90E−10 |
| 2-2D11 | 1.389 | 5.34E−09 | 2-4F9 | 2.639 | 9.33E−10 |
| 2-2E1 | 2.097 | 2.07E−08 | 2-4G1 | 2.075 | 1.44E−08 |
| 2-2E7 | 2.291 | 1.57E−08 | 2-4G4 | 3.404 | 9.20E−09 |
| 2-2E11 | 0.737 | 2.68E−09 | 2-4G10 | 3.522 | n/a |
| 2-2F5 | 2.029 | 6.32E−10 | 2-4G12 | 1.229 | 1.01E−08 |
| 2-2F10 | 2.330 | 1.29E−10 | 2-4H8 | 0.811 | 3.42E−08 |
| 2-5A2 | 0.870 | 5.84E−11 | 2-7D12 | 1.345 | 1.32E−08 |
| 2-5A7 | 3.467 | 2.16E−11 | 2-7E5 | 3.219 | 3.38E−10 |
| 2-5A12 | 1.786 | n/a | 2-7E11 | 3.219 | 6.39E−11 |
| 2-5B4 | 3.457 | 2.94E−10 | 2-7F4 | 3.037 | 2.43E−10 |
| 2-5C3 | 1.333 | 2.21E−09 | 2-7G1 | 3.377 | 8.21E−11 |
| 2-5C4 | 2.261 | 2.60E−11 | 2-7G8 | 3.400 | 3.10E−10 |
| 2-5D9 | 3.492 | 5.44E−10 | 2-7H8 | 2.908 | 2.11E−10 |
| 2-5F8 | 3.478 | 1.50E−09 | 2-8A2 | 1.625 | 8.17E−11 |
| 2-5F10 | 3.349 | 2.78E−11 | 2-8B2 | 3.076 | 4.12E−09 |
| 2-5H1 | 1.054 | 1.60E−08 | 2-8B9 | 1.388 | 3.00E−10 |
| 2-6A11 | 3.129 | 8.70E−09 | 2-8B12 | 3.470 | 7.92E−11 |
| 2-6B2 | 1.691 | 7.61E−09 | 2-8C6 | 1.471 | 2.44E−09 |
| 2-6B9 | 1.442 | 1.00E−08 | 2-8C8 | 1.505 | 1.33E−10 |
| 2-6B12 | 0.890 | n/a | 2-8C12 | 3.045 | 4.86E−09 |
| 2-6C8 | 2.941 | n/a | 2-8D6 | 3.459 | 1.84E−09 |
| 2-6C10 | 1.429 | n/a | 2-8E2 | 3.123 | 4.05E−09 |
| 2-6D3 | 1.410 | 2.57E−08 | 2-8E3 | 3.376 | 1.85E−09 |
| 2-6D5 | 3.221 | 1.87E−08 | 2-8E7 | 3.147 | 1.27E−09 |
| 2-6D9 | 3.315 | 2.24E−11 | 2-8E9 | 1.538 | 2.36E−09 |
| 2-6D11 | 1.561 | 7.11E−01 | 2-8F8 | 1.360 | 3.00E−09 |
| 2-6E12 | 2.136 | 7.87E−09 | 2-8G1 | 0.832 | 1.82E−09 |
| 2-6F6 | 1.547 | 7.72E−09 | 2-8G8 | 3.226 | 9.46E−10 |
| 2-6F7 | 1.041 | 2.13E−09 | 2-8H3 | 1.432 | 3.52E−09 |
| 2-6F9 | 1.120 | 6.31E−11 | 2-9B1 | 2.342 | 3.68E−11 |
| 2-6G4 | 1.976 | 1.69E−09 | 2-9B2 | 1.300 | 1.60E−10 |
| 2-6G5 | 1.150 | 7.90E−10 | 2-9B4 | 1.447 | 1.29E−09 |
| 2-6G9 | 1.308 | 3.21E−09 | 2-9C6 | 2.188 | 7.38E−11 |
| 2-6H2 | 1.952 | 2.09E−10 | 2-9D3 | 1.416 | 1.15E−11 |
| 2-7A4 | 1.409 | n/a | 2-9D7 | 2.131 | 2.78E−10 |
| 2-7A7 | 1.119 | 4.22E−10 | 2-9E1 | 3.381 | n/a |
| 2-7B6 | 1.392 | 5.81E−08 | 2-9E9 | 3.319 | 1.50E−09 |
| 2-7B11 | 0.880 | 1.80E−08 | 2-9E11 | 0.441 | n/a |
| 2-7C4 | 1.164 | 1.11E−09 | 2-9F2 | 3.069 | 6.58E−09 |
| 2-7C12 | 3.068 | 7.62E−09 | 2-9F3 | 1.526 | n/a |
| 2-7D9 | 2.913 | 2.90E−09 | 2-9F7 | 3.156 | 1.66E−09 |
| 2-9F9 | 3.496 | 1.57E−09 | 2-12C8 | 1.775 | 2.07E−10 |
| 2-9G1 | 0.673 | 2.51E−08 | 2-12D4 | 1.019 | 1.77E−08 |
| 2-9G2 | 0.931 | 1.54E−10 | 2-12D6 | 2.908 | 2.79E−09 |
| 2-9G3 | 1.071 | 7.79E−11 | 2-12E1 | 1.646 | 8.65E−09 |
| 2-9G5 | 3.309 | n/a | 2-12E8 | 1.756 | 4.03E−11 |
| 2-9G6 | 2.711 | 8.25E−10 | 2-12F3 | 3.412 | n/a |
| 2-9G8 | 3.161 | 1.34E−09 | 2-12G2 | 3.070 | n/a |
| 2-9H9 | 1.150 | n/a | 2-12G8 | 1.570 | 2.27E−08 |
| 2-10A2 | 1.309 | n/a | 2-12H7 | 1.398 | n/a |
| 2-10A3 | 2.227 | 9.81E−09 | 2-13A11 | 2.106 | 2.92E−08 |
| 2-10B7 | 3.105 | 7.32E−09 | 2-13B10 | 3.154 | 1.02E−08 |
| 2-10B12 | 1.916 | 6.52E−09 | 2-13B7 | 3.106 | 1.71E−09 |
| 2-10C5 | 2.915 | 4.45E−10 | 2-13D8 | 1.995 | n/a |
| 2-10D4 | 3.169 | 4.27E−10 | 2-13D12 | 3.296 | 6.08E−10 |
| 2-10D8 | 2.275 | 2.04E−08 | 2-13F3 | 1.995 | 2.49E−08 |
| 2-10E1 | 2.851 | 1.17E−09 | 2-13G9 | 2.981 | 4.08E−09 |
| 2-10E3 | 1.575 | 4.83E−10 | 2-13H10 | 3.277 | 1.06E−09 |
| 2-10E10 | 3.343 | 4.12E−09 | 2-14B5 | 3.319 | 7.12E−09 |
| 2-10F11 | 2.399 | 1.73E−10 | 2-14B10 | 3.396 | 1.80E−09 |
| 2-10G2 | 2.149 | 3.46E−11 | 2-14B12 | 1.378 | 2-14B12 |
| 2-10G7 | 0.905 | 9.62E−09 | 2-14C8 | 3.387 | 2.46E−09 |
| 2-10H2 | 1.967 | 1.22E−09 | 2-14C10 | 3.401 | 1.47E−09 |
| 2-10H4 | 1.893 | 4.47E−10 | 2-14D5 | 3.397 | 8.04E−11 |
| 2-11A11 | 0.550 | 9.48E−10 | 2-14E7 | 1.283 | 2.20E−08 |
| 2-11B8 | 2.753 | 1.06E−08 | 2-14E9 | 2.309 | 2.75E−10 |
| 2-11C10 | 3.102 | 2.16E−09 | 2-14F3 | 2.398 | 2.12E−09 |
| 2-11D2 | 1.781 | 7.98E−09 | 2-14G2 | 3.347 | 1.94E−09 |
| 2-11D12 | 2.739 | 8.98E−09 | 2-14G4 | 2.858 | 1.91E−08 |
| 2-11E4 | 3.028 | 1.31E−08 | 2-14G5 | 3.219 | 3.14E−10 |
| 2-11E10 | 2.775 | 1.34E−07 | 2-14G6 | 3.362 | n/a |
| 2-11G6 | 3.434 | 8.86E−09 | 2-14G7 | 3.292 | 3.30E−09 |
| 2-11H3 | 3.337 | 6.85E−10 | 2-14G10 | 3.339 | 2.59E−11 |
| 2-12A2 | 2.771 | 2.89E−10 | 2-14H4 | 1.260 | 2.03E−10 |
| 2-12B6 | 1.999 | 2.25E−09 | 2-15A1 | 0.665 | n/a |
| 2-12C7 | 3.247 | 5.77E−10 | 2-15A9 | 2.077 | 1.21E−09 |
| 2-15B2 | 2.349 | 4.41E−10 | 2-18C4 | 0.552 | n/a |
| 2-15B12 | 1.220 | 1.91E−09 | 3-1A7 | 1.189 | 8.99E−11 |
| 2-15C6 | 1.586 | 1.36E−09 | 3-1A12 | 2.030 | 4.55E−09 |
| 2-15D1 | 2.314 | 2.26E−10 | 3-1 E6 | 1.376 | 1.24E−08 |
| 2-15D9 | 2.587 | n/a | 3-1 E8 | 2.019 | 1.81E−10 |
| 2-15D10 | 1.710 | 5.35E−09 | 3-2A8 | 1.909 | 4.10E−11 |
| 2-15F2 | 2.386 | 4.79E−10 | 3-2C5 | 1.163 | 1.54E−08 |
| 2-15F7 | 3.248 | 4.57E−09 | 3-2C9 | 1.220 | 5.63E−09 |
| 2-15F8 | 2.262 | 2.01E−09 | 3-2D7 | 1.874 | 2.80E−08 |
| 2-15F11 | 3.170 | n/a | 3-2E10 | 1.341 | 1.99E−08 |
| 2-15G5 | 2.657 | 5.03E−09 | 3-2E12 | 1.786 | 5.81E−09 |
| 2-15G6 | 1.002 | 5.32E−09 | 3-2G7 | 2.148 | 7.65E−11 |
| 2-15H6 | 3.265 | 6.06E−09 | 3-3A1 | 1.356 | 5.57E−09 |
| 2-16A6 | 2.066 | 1.11E−08 | 3-3A3 | 1.406 | 2.42E−08 |
| 2-16A8 | 3.313 | 1.90E−09 | 3-3B5 | 1.993 | 2.37E−10 |
| 2-16C7 | 3.428 | 1.62E−11 | 3-3B8 | 2.349 | 3.37E−10 |
| 2-16D1 | 3.429 | 1.67E−09 | 3-3C8 | 1.424 | 6.19E−09 |
| 2-16D2 | 2.340 | 1.06E−10 | 3-3D3 | 1.476 | 1.09E−09 |
| 2-16D9 | 3.189 | 5.14E−09 | 3-3D12 | 1.566 | 3.81E−09 |
| 2-16D10 | 3.503 | 3.83E−09 | 3-3E9 | 0.826 | 1.77E−09 |
| 2-16G5 | 3.030 | 3.64E−09 | 3-3E10 | 1.337 | 1.38E−08 |
| 2-16G6 | 3.157 | 1.13E−08 | 3-3F5 | 1.088 | 2.22E−09 |
| 2-17B4 | 3.179 | 1.51E−08 | 3-3G11 | 1.262 | 2.65E−10 |
| 2-17B5 | 3.325 | n/a | 3-4D12 | 1.423 | 5.11E−09 |
| 2-17C10 | 3.368 | 4.46E−11 | 3-4F2 | 1.425 | 8.81E−10 |
| 2-17E1 | 3.399 | 7.38E−09 | 3-4F3 | 1.037 | 3.84E−09 |
| 2-16E11 | 2.439 | 1.61E−10 | 3-4F9 | 1.298 | 5.36E−11 |
| 2-17E12 | 3.293 | 1.69E−10 | 3-5B7 | 1.488 | 9.90E−09 |
| 2-17F4 | 1.887 | 1.78E−08 | 3-5B10 | 0.825 | 1.03E−08 |
| 2-17F9 | 1.964 | 4.79E−09 | 3-5E10 | 0.658 | 1.52E−09 |
| 2-17G9 | 0.973 | n/a | 3-5F2 | 1.077 | 2.68E−09 |
| 2-18A4 | 2.149 | n/a | 3-5F7 | 0.596 | 1.90E−09 |
| 2-18B3 | 2.401 | 1.42E−09 | 3-5F8 | 1.383 | 8.43E−09 |
| 2-18B4 | 1.832 | n/a | 3-5G5 | 0.741 | 4.28E−10 |
| 2-18B10 | 1.518 | 1.74E−09 | 3-5G6 | 1.438 | 1.67E−09 |
| 3-5G9 | 1.411 | 1.58E−09 | 3-11B1 | 1.214 | 5.28E−09 |
| 3-5H10 | 1.904 | 4.58E−09 | 3-11B4 | 1.619 | 2.28E−08 |
| 3-6A2 | 0.584 | 2.64E−09 | 3-11B6 | 0.610 | 4.49E−09 |
| 3-6B11 | 1.739 | 1.26E−09 | 3-11C1 | 0.740 | 8.54E−10 |
| 3-6C3 | 1.819 | 1.85E−08 | 3-11 E10 | 2.665 | 1.80E−09 |
| 3-6D12 | 0.903 | 4.61E−09 | 3-11H2 | 0.670 | 2.62E−09 |
| 3-6F2 | 0.618 | n/a | 3-11H7 | 1.397 | 1.28E−08 |
| 3-7G1 | 0.699 | 2.95E−09 | 3-12B10 | 1.430 | 8.89E−10 |
| 3-6G3 | 1.711 | 1.34E−09 | 3-12B12 | 3.439 | 1.65E−11 |
| 3-6G4 | 1.957 | 3.48E−09 | 3-12D11 | 1.469 | 2.23E−09 |
| 3-6H5 | 2.091 | 4.73E−09 | 3-12E2 | 1.413 | 6.18E−08 |
| 3-7B3 | 2.345 | 9.71E−10 | 3-12E3 | 1.333 | 6.77E−10 |
| 3-7D1 | 0.508 | 1.58E−08 | 3-13E3 | 1.370 | 8.07E−10 |
| 3-7G3 | 0.635 | 6.61E−10 | 3-13H5 | 0.570 | 1.12E−09 |
| 3-8A7 | 2.336 | 3.29E−09 | 3-12H6 | 1.405 | 1.89E−09 |
| 3-8B9 | 0.975 | 5.30E−09 | 3-13H10 | 1.292 | 2.14E−10 |
| 3-8C3 | 0.500 | 5.39E+00 | 3-13D11 | 0.721 | n/a |
| 3-8D1 | 1.086 | 6.72E−09 | 3-13F10 | 0.976 | 3.85E−05 |
| 3-8E2 | 1.193 | 1.20E−09 | 3-14E9 | 3.203 | 4.69E−11 |
| 3-8E12 | 0.804 | 6.11E−10 | 5-1 A4 | 3.307 | 3.76E−09 |
| 3-8F11 | 1.418 | 1.18E−10 | 5-1 A6 | 3.255 | n/a |
| 3-9A2 | 0.707 | 3.41E−10 | 5-1B9 | 3.389 | 1.56E−09 |
| 3-9A10 | 1.198 | 4.07E−09 | 5-1C1 | 3.241 | 4.90E−09 |
| 3-9A12 | 3.128 | 2.53E−08 | 5-1C10 | 3.173 | 1.40E−08 |
| 3-9C4 | 1.987 | 3.49E−09 | 5-1D5 | 3.338 | 3.57E−10 |
| 3-9D7 | 0.533 | 6.00E−09 | 5-1 E3 | 3.272 | 1.60E−09 |
| 3-9D12 | 0.481 | 4.77E−08 | 5-1 E4 | 1.991 | n/a |
| 3-9F5 | 2.187 | 3.95E−09 | 5-1F3 | 0.401 | n/a |
| 3-9F6 | 1.095 | 7.39E−09 | 5-1G1 | 3.132 | n/a |
| 3-9G1 | 1.761 | 1.05E−09 | 5-1G12 | 0.572 | n/a |
| 3-9H1 | 2.060 | 2.92E−10 | 5-1H4 | 3.345 | 3.87E−11 |
| 3-9H4 | 3.316 | 4.22E−11 | 5-1H6 | 2.346 | 3.67E−11 |
| 3-10E1 | 1.304 | 9.62E−09 | 5-2A7 | 1.673 | 5.26E−10 |
| 3-10G11 | 0.997 | 2.34E−10 | 5-2A12 | 1.380 | 2.41E−10 |
| 3-11A10 | 1.330 | 1.73E−09 | 5-2B1 | 1.969 | 9.69E−10 |
| 5-2B5 | 1.813 | 2.65E−08 | 5-5B6 | 3.243 | 1.48E−11 |
| 5-2B9 | 2.155 | 1.66E−08 | 5-5B7 | 3.277 | 3.46E−09 |
| 5-2B12 | 2.970 | 3.38E−10 | 5-5B10 | 3.257 | n/a |
| 5-2C8 | 0.770 | n/a | 5-5D2 | 3.178 | 8.59E−09 |
| 5-2D1 | 3.311 | 1.30E−10 | 5-5D6 | 3.393 | 3.33E−09 |
| 5-2D8 | 3.006 | 4.18E−11 | 5-5D7 | 1.577 | 6.81E−08 |
| 5-2D11 | 3.291 | 3.44E−09 | 5-5D10 | 2.053 | 4.20E−10 |
| 5-2F5 | 0.714 | 4.51E−09 | 5-5E5 | 2.101 | n/a |
| 5-2F8 | 3.273 | 8.03E−09 | 5-5E8 | 2.090 | 5.95E−09 |
| 5-2F12 | 3.490 | 6.43E−11 | 5-5E9 | 2.076 | 4.86E−11 |
| 5-2G1 | 3.436 | n/a | 5-5E10 | 3.253 | 7.24E−11 |
| 5-2G3 | 0.726 | 5.53E−09 | 5-5E12 | 2.958 | 9.40E−09 |
| 5-2G7 | 3.388 | 4.33E−11 | 5-5F1 | 3.237 | 7.39E−11 |
| 5-2H8 | 3.445 | 1.04E−10 | 5-5F8 | 3.331 | 8.91E−10 |
| 5-3A11 | 3.234 | 1.91E−09 | 5-5G9 | 3.282 | n/a |
| 5-3D11 | 3.340 | 1.31E−10 | 5-5H4 | 3.281 | 3.00E−10 |
| 5-3E3 | 3.428 | 8.02E−09 | 5-5H5 | 3.398 | 9.10E−10 |
| 5-3E8 | 2.718 | 4.42E−09 | 5-5H10 | 3.468 | 1.02E−08 |
| 5-3G8 | 2.276 | 3.95E−10 | 5-6A2 | 3.414 | 1.26E−09 |
| 5-3G10 | 0.404 | 1.07E−09 | 5-6A11 | 3.393 | 5.32E−09 |
| 5-3H6 | 2.198 | 5.09E−09 | 5-6B6 | 2.200 | n/a |
| 5-3H8 | 2.455 | 4.16E−10 | 5-6C12 | 3.131 | n/a |
| 5-4A2 | 1.343 | 3.59E−09 | 5-6D1 | 3.387 | 5.83E−11 |
| 5-4A7 | 1.621 | 3.97E−10 | 5-6E10 | 3.274 | 2.78E−09 |
| 5-4B7 | 3.342 | 1.03E−09 | 5-6E11 | 3.138 | 1.27E−09 |
| 5-4B8 | 3.187 | 9.30E−09 | 5-6H2 | 3.479 | 5.24E−10 |
| 5-4D3 | 3.028 | 4.99E−09 | 5-7A4 | 1.285 | 8.03E−11 |
| 5-4D6 | 2.903 | 1.08E−10 | 5-7B3 | 2.266 | 3.36E−11 |
| 5-4E1 | 3.147 | 2.46E−09 | 5-7B6 | 2.223 | 4.07E−09 |
| 5-4F2 | 0.458 | 1.55E−08 | 5-7B11 | 2.220 | 2.27E−09 |
| 5-4F5 | 3.332 | 4.46E−09 | 5-7C2 | 1.289 | 1.93E−09 |
| 5-4F6 | 3.326 | 4.19E−09 | 5-7C12 | 2.158 | 3.82E−09 |
| 5-4G3 | 3.311 | 1.12E−09 | 5-7D5 | 3.419 | 1.50E−09 |
| 5-4H6 | 3.295 | 1.36E−09 | 5-7E2 | 3.296 | 7.74E−09 |
| 5-5A2 | 3.242 | 5.35E−09 | 5-7E9 | 3.237 | 1.27E−08 |
| 5-7E10 | 3.356 | 2.16E−10 | 5-10B7 | 1.304 | 1.66E−09 |
| 5-7E12 | 3.140 | 5.48E−09 | 5-10C4 | 3.338 | 2.78E−09 |
| 5-7F3 | 3.304 | 9.07E−09 | 5-10C10 | 2.945 | 9.67E−09 |
| 5-7F7 | 3.416 | 5.82E−09 | 5-10E5 | 3.460 | 5.92E−11 |
| 5-7F9 | 3.488 | 4.10E−10 | 5-10F3 | 2.571 | 1.48E−10 |
| 5-7G6 | 1.880 | 1.47E−09 | 5-10F8 | 1.900 | n/a |
| 5-7G11 | 2.903 | 2.69E−09 | 5-10G2 | 2.380 | 1.06E−10 |
| 5-7H10 | 3.254 | 1.10E−09 | 5-10G7 | 1.581 | 6.09E−11 |
| 5-8A9 | 2.884 | 4.02E−11 | 5-10H5 | 2.719 | n/a |
| 5-8B1 | 2.998 | 4.20E−10 | 5-11A5 | 3.474 | 1.90E−10 |
| 5-8B8 | 3.334 | 3.71E−10 | 5-11B5 | 3.383 | 3.58E−09 |
| 5-8B10 | 3.348 | 4.90E−09 | 5-11B12 | 2.756 | 1.86E−09 |
| 5-8C5 | 3.411 | 5.83E−09 | 5-11D7 | 3.386 | 1.72E−09 |
| 5-8C8 | 3.386 | 8.25E−09 | 5-11D10 | 3.371 | 4.48E−09 |
| 5-8C11 | 2.217 | 1.37E−09 | 5-11E4 | 3.178 | 1.24E−09 |
| 5-8D7 | 3.400 | 7.66E−09 | 5-11F4 | 1.492 | n/a |
| 5-8F9 | 1.958 | 4.29E−09 | 5-11G10 | 3.322 | 9.05E−11 |
| 5-8F11 | 1.514 | 1.56E−09 | 5-11H5 | 3.442 | 9.53E−11 |
| 5-8G9 | 2.206 | 5.38E−09 | 5-11H9 | 0.574 | n/a |
| 5-8G11 | 1.656 | 3.96E−09 | 5-12A3 | 2.704 | n/a |
| 5-8H5 | 2.219 | 1.84E−09 | 5-12B3 | 3.328 | 1.64E−10 |
| 5-9A11 | 1.480 | 2.01E−08 | 5-12B5 | 3.238 | 5.97E−10 |
| 5-9B9 | 3.421 | 1.94E−09 | 5-12C1 | 3.337 | 1.81E−09 |
| 5-9B11 | 3.330 | 4.44E−09 | 5-12C5 | 3.366 | 4.68E−11 |
| 5-9C9 | 3.073 | 5.63E−09 | 5-12D1 | 2.095 | 3.68E−11 |
| 5-9C10 | 2.723 | 3.14E−11 | 5-12D2 | 1.741 | 3.32E−11 |
| 5-9C12 | 0.778 | 6.37E−11 | 5-12D10 | 2.032 | 1.44E−09 |
| 5-9D5 | 2.070 | 3.85E−09 | 5-12E1 | 2.282 | 1.57E−10 |
| 5-9F7 | 3.515 | 3.43E−11 | 5-12E11 | 2.189 | 5.94E−11 |
| 5-9F10 | 2.122 | 2.66E−09 | 5-12F3 | 2.099 | 1.81E−09 |
| 5-9G1 | 3.240 | 1.04E−08 | 5-12G4 | 1.992 | 2.28E−09 |
| 5-9G5 | 3.424 | 4.13E−09 | 5-12G6 | 3.198 | 1.65E−10 |
| 5-9G10 | 3.503 | 3.51E−09 | 5-12H1 | 2.370 | 1.76E−08 |
| 5-9H1 | 3.438 | 2.41E−09 | 5-12H2 | 3.000 | 7.08E−09 |
| 5-9H7 | 3.341 | 1.24E−09 | 5-13A2 | 1.948 | 1.41E−09 |
| 5-13A3 | 3.218 | n/a | 9-1B9 | 2.150 | 2.18E−08 |
| 5-13A9 | 2.851 | n/a | 9-1C2 | 2.083 | 1.22E−09 |
| 5-13C1 | 0.634 | n/a | 9-1C4 | 2.103 | 9.96E−11 |
| 5-13C4 | 0.502 | 3.63E−09 | 9-1C11 | 2.085 | 1.66E−09 |
| 5-13D3 | 3.405 | 4.72E−10 | 9-1 D6 | 2.115 | 2.78E−09 |
| 5-13D6 | 3.399 | 2.02E−10 | 9-1D11 | 1.581 | 1.29E−08 |
| 5-13E6 | 3.436 | 9.20E−11 | 9-1 E6 | 2.118 | 8.23E−09 |
| 5-13E8 | 3.379 | 1.65E−09 | 9-1 E8 | 1.899 | 1.42E−09 |
| 5-13E12 | 3.422 | 3.76E−09 | 9-1E10 | 0.420 | n/a |
| 5-13F5 | 2.353 | 2.06E−09 | 9-1F2 | 2.650 | 1.83E−11 |
| 5-13F7 | 3.138 | 5.65E−09 | 9-1F7 | 2.512 | 1.18E−10 |
| 5-13G5 | 0.595 | 4.20E−08 | 9-1F8 | 2.490 | 1.13E−09 |
| 5-13G8 | 3.068 | 6.30E−09 | 9-1F10 | 2.201 | 3.67E−09 |
| 5-13H4 | 3.420 | 3.27E−09 | 9-1G1 | 2.531 | 1.61E−09 |
| 5-13H9 | 3.375 | 7.04E−11 | 9-1G3 | 1.594 | 1.94E−08 |
| 5-14A1 | 1.764 | n/a | 9-1G8 | 1.512 | 8.25E−09 |
| 5-14B3 | 2.237 | 3.40E−09 | 9-1H5 | 2.242 | n/a |
| 5-14B9 | 2.237 | 1.53E−09 | 9-1H7 | 2.282 | n/a |
| 5-14C5 | 1.113 | n/a | 9-1H9 | 2.047 | n/a |
| 5-14C8 | 2.448 | 1.71E−10 | 9-2A4 | 1.840 | 1.53E−08 |
| 5-14D5 | 2.581 | 1.81E−09 | 9-2A8 | 1.713 | 7.22E−09 |
| 5-14D9 | 0.626 | 4.18E−10 | 9-2B6 | 2.357 | 1.33E−09 |
| 5-14E2 | 3.240 | 1.07E−08 | 9-2B8 | 1.922 | 1.33E−09 |
| 5-14E10 | 3.260 | 3.63E−09 | 9-2B10 | 2.393 | 4.85E−09 |
| 5-14E12 | 3.321 | 3.86E−09 | 9-2B11 | 2.475 | n/a |
| 5-14G1 | 3.321 | 4.54E−09 | 9-2B12 | 2.515 | 4.55E−09 |
| 5-14G4 | 1.757 | 7.97E−09 | 9-2C4 | 2.423 | 2.22E−10 |
| 5-14G5 | 1.215 | n/a | 9-2C9 | 2.314 | 4.37E−08 |
| 9-1A3 | 1.734 | 2.99E−08 | 9-2C11 | 1.175 | 7.89E−08 |
| 9-1 A9 | 0.653 | n/a | 9-2D9 | 2.130 | 1.73E−09 |
| 9-1A11 | 1.888 | 1.36E−08 | 9-2D10 | 2.353 | 3.65E−09 |
| 9-1B2 | 1.582 | 5.84E−11 | 9-2D12 | 2.509 | 3.77E−09 |
| 9-1B3 | 1.730 | 1.24E−10 | 9-2E3 | 2.429 | 1.16E−08 |
| 9-1B5 | 1.555 | 6.82E−09 | 9-2E6 | 2.505 | n/a |
| 9-1B8 | 0.861 | n/a | 9-2E7 | 2.145 | 1.60E−09 |
| 9-2E10 | 1.656 | 5.73E−10 | 9-4D1 | 0.515 | n/a |
| 9-2F4 | 2.401 | 4.55E−10 | 9-4D6 | 2.403 | n/a |
| 9-2F6 | 1.388 | 5.81E−08 | 9-4D7 | 2.877 | 5.76E−11 |
| 9-2F7 | 2.592 | 4.33E−10 | 9-4D8 | 1.524 | 7.85E−09 |
| 9-2F8 | 0.607 | n/a | 9-4E1 | 2.532 | 1.08E−08 |
| 9-2G4 | 2.502 | 1.55E−08 | 9-4E3 | 1.235 | n/a |
| 9-2G8 | 2.558 | 2.00E−09 | 9-4E5 | 1.342 | n/a |
| 9-2G10 | 1.454 | 2.00E−09 | 9-4E10 | 1.981 | 1.44E−08 |
| 9-2H3 | 2.806 | n/a | 9-4E12 | 2.481 | 6.61E−09 |
| 9-2H6 | 1.401 | 6.43E−10 | 9-4F1 | 1.885 | 2.80E−08 |
| 9-2H7 | 3.201 | 9.77E−12 | 9-4F2 | 2.357 | n/a |
| 9-2H8 | 0.599 | n/a | 9-4F3 | 1.611 | n/a |
| 9-3A7 | 2.605 | 3.13E−09 | 9-4F4 | 3.245 | 1.22E−09 |
| 9-3B3 | 1.825 | 4.12E−10 | 9-4G4 | 2.691 | n/a |
| 9-3B4 | 1.899 | 2.33E−09 | 9-4G5 | 1.732 | 1.06E−08 |
| 9-3C1 | 2.591 | 3.36E−09 | 9-4G9 | 0.809 | n/a |
| 9-3C4 | 2.310 | 4.37E−11 | 9-4H1 | 2.065 | 6.92E−09 |
| 9-3C8 | 1.292 | 6.25E−09 | 9-4H4 | 2.944 | 4.14E−10 |
| 9-3D2 | 1.824 | 7.61E−09 | 9-4H5 | 2.289 | 3.63E−08 |
| 9-3D4 | 2.712 | 3.22E−10 | 9-4H6 | 2.854 | 1.96E−09 |
| 9-3D12 | 1.783 | 2.30E−09 | 9-5A1 | 2.428 | 2.67E−08 |
| 9-3E5 | 2.824 | 2.11E−09 | 9-5A2 | 0.587 | n/a |
| 9-3E9 | 1.830 | 1.13E−08 | 9-5A3 | 3.116 | 3.01E−09 |
| 9-3F2 | 2.534 | 1.42E−08 | 9-5A4 | 2.751 | 3.01E−09 |
| 9-3F7 | 2.667 | 2.89E−11 | 9-5A7 | 0.756 | n/a |
| 9-3F8 | 1.740 | 3.51E−09 | 9-5A9 | 0.525 | n/a |
| 9-3G1 | 2.605 | 5.45E−11 | 9-5A10 | 2.943 | 1.62E−09 |
| 9-3H5 | 2.358 | 4.80E−11 | 9-5A11 | 2.717 | 1.09E−08 |
| 9-3H9 | 2.132 | 4.44E−10 | 9-5A12 | 1.900 | 1.55E−09 |
| 9-4A6 | 2.674 | 1.28E−09 | 9-5B1 | 2.125 | 2.30E−08 |
| 9-4A10 | 1.343 | n/a | 9-5B2 | 1.622 | n/a |
| 9-4B3 | 3.007 | 2.00E−08 | 9-5B4 | 2.995 | 3.82E−09 |
| 9-4B5 | 0.699 | 6.63E−09 | 9-5B5 | 2.621 | 1.33E−08 |
| 9-4B10 | 3.041 | 1.53E−09 | 9-5B10 | 2.836 | 5.48E−11 |
| 9-4C5 | 0.725 | n/a | 9-5C12 | 1.126 | n/a |
| 9-5D2 | 2.959 | 1.80E−09 | 9-6H10 | 1.810 | n/a |
| 9-5D12 | 0.972 | 1.56E−09 | 9-7A4 | 3.124 | 7.35E−10 |
| 9-5E1 | 1.913 | 1.70E−08 | 9-7A11 | 3.131 | 3.77E−09 |
| 9-5E3 | 2.513 | 3.16E−09 | 9-7B4 | 0.969 | n/a |
| 9-5E4 | 2.874 | 6.71E−11 | 9-7B6 | 3.201 | 9.36E−11 |
| 9-5E10 | 1.459 | n/a | 9-7B8 | 0.991 | 3.81E−09 |
| 9-5F4 | 1.867 | n/a | 9-7B9 | 2.427 | 6.56E−09 |
| 9-5F7 | 2.144 | n/a | 9-7B11 | 1.563 | 2.41E−08 |
| 9-5F9 | 2.735 | 1.38E−08 | 9-7B12 | 2.599 | 3.90E−09 |
| 9-5G9 | 0.976 | 5.89E−09 | 9-7C4 | 2.871 | 1.28E−08 |
| 9-5G12 | 3.238 | 3.49E−11 | 9-7C7 | 2.564 | n/a |
| 9-5H2 | 3.176 | n/a | 9-7D3 | 1.538 | 1.24E−09 |
| 9-5H3 | 2.048 | 2.29E−09 | 9-7D4 | 2.418 | 1.87E−08 |
| 9-5H6 | 0.465 | 2.94E−09 | 9-7D12 | 2.103 | 3.50E−09 |
| 9-5H10 | 2.537 | 3.58E−09 | 9-7E1 | 2.634 | n/a |
| 9-6B2 | 2.487 | 1.18E−08 | 9-7E7 | 2.174 | 1.09E−08 |
| 9-6B10 | 2.733 | 9.02E−11 | 9-7E8 | 2.752 | 3.58E−09 |
| 9-6C1 | 3.131 | 8.24E−11 | 9-7E10 | 2.336 | 1.24E−10 |
| 9-6D1 | 1.278 | n/a | 9-7E11 | 2.347 | n/a |
| 9-6D4 | 2.619 | 8.70E−09 | 9-7F1 | 2.465 | 1.95E−09 |
| 9-6D7 | 2.350 | 8.98E−09 | 9-7F5 | 1.629 | n/a |
| 9-6D8 | 1.443 | 1.06E−10 | 9-7F8 | 2.888 | 3.44E−11 |
| 9-6D10 | 2.181 | 3.29E−09 | 9-7F11 | 2.403 | 1.64E−09 |
| 9-6E3 | 3.056 | 5.72E−11 | 9-7F12 | 1.874 | n/a |
| 9-6E7 | 2.663 | 7.55E−09 | 9-7G5 | 2.733 | 1.86E−10 |
| 9-6E9 | 2.773 | 3.02E−09 | 9-7G7 | 0.758 | 2.52E−09 |
| 9-6E10 | 2.337 | 6.31E−08 | 9-7G10 | 1.538 | n/a |
| 9-6E12 | 2.982 | 5.00E−09 | 9-7G11 | 2.191 | 4.65E−11 |
| 9-6F4 | 2.605 | 2.10E−08 | 9-8A2 | 2.909 | n/a |
| 9-6F11 | 3.061 | 1.55E−09 | 9-8A3 | 0.461 | 2.48E−09 |
| 9-6G2 | 3.126 | 2.10E−10 | 9-8A4 | 0.654 | n/a |
| 9-6G7 | 0.632 | n/a | 9-8B1 | 0.414 | 1.23E−08 |
| 9-6G9 | 1.380 | n/a | 9-8B3 | 2.611 | 1.52E−08 |
| 9-6H1 | 2.808 | 5.55E−10 | 9-8C4 | 2.978 | 1.27E−09 |
| 9-6H2 | 3.076 | 1.74E−09 | 9-8C6 | 2.866 | 7.54E−11 |
| 9-8C7 | 3.238 | 1.98E−09 | 9-9E3 | 1.094 | |
| 9-8C11 | 2.010 | 7.20E−09 | 9-9F5 | 0.413 | n/a |
| 9-8C12 | 1.462 | 8.77E−09 | 9-9F8 | 2.093 | n/a |
| 9-8D1 | 2.429 | 3.15E−09 | 9-9F11 | 3.013 | 6.74E−11 |
| 9-8D3 | 1.991 | 7.66E−09 | 9-9G5 | 3.199 | 3.58E−09 |
| 9-8D5 | 0.481 | 6.32E−09 | 9-10A1 | 3.022 | 9.20E−11 |
| 9-8D6 | 2.676 | 9.21E−09 | 9-10B1 | 0.682 | 1.03E−06 |
| 9-8E4 | 2.727 | 1.61E−08 | 9-10C1 | 3.043 | 7.30E−11 |
| 9-8E5 | 2.046 | 2.32E−09 | 9-10C2 | 1.398 | 1.20E−08 |
| 9-8F1 | 2.804 | n/a | 9-10C8 | 2.780 | 1.69E−09 |
| 9-8F2 | 2.777 | 1.09E−09 | 9-10D4 | 2.793 | 3.41E−09 |
| 9-8F3 | 1.656 | n/a | 9-10E2 | 1.797 | 8.28E−09 |
| 9-8F4 | 1.499 | 5.07E−07 | 9-10E3 | 3.046 | 2.10E−09 |
| 9-8F7 | 2.597 | 1.32E−08 | 9-10E6 | 1.204 | n/a |
| 9-8G1 | 3.038 | n/a | 9-10E8 | 2.592 | 3.80E−11 |
| 9-8G2 | 1.265 | n/a | 9-10E10 | 1.943 | 2.06E−08 |
| 9-8G5 | 0.965 | n/a | 9-10F5 | 2.263 | 1.96E−08 |
| 9-8G7 | 2.685 | 6.41E−10 | 9-10F11 | 2.835 | 3.32E−09 |
| 9-8G8 | 2.409 | n/a | 9-10G1 | 2.382 | 1.02E−09 |
| 9-8G9 | 1.747 | 1.65E−08 | 9-10G4 | 2.921 | 1.48E−10 |
| 9-8G10 | 2.132 | 8.41E−10 | 9-10G5 | 3.218 | 2.58E−09 |
| 9-8H4 | 2.015 | 2.04E−10 | 9-10G12 | 1.550 | 7.32E−09 |
| 9-8H7 | 2.581 | 1.07E−09 | 9-10H6 | 1.215 | n/a |
| 9-8H8 | 0.564 | 1.40E−08 | 9-10H9 | 1.962 | 4.67E−11 |
| 9-9A2 | 2.441 | 1.47E−07 | 9-11A4 | 2.167 | 6.83E−11 |
| 9-9A3 | 1.073 | 3.02E−08 | 9-11A5 | 2.891 | 1.40E−09 |
| 9-9B4 | 2.175 | 5.94E−09 | 9-11A6 | 2.425 | n/a |
| 9-9B8 | 2.275 | 1.73E−05 | 9-11A7 | 2.911 | n/a |
| 9-9B9 | 2.443 | 1.70E−08 | 9-11A10 | 3.336 | 4.84E−09 |
| 9-9C2 | 2.816 | 1.27E−09 | 9-11B5 | 1.887 | 1.31E−08 |
| 9-9C4 | 1.990 | 2.17E−09 | 9-11C6 | 2.241 | n/a |
| 9-9C5 | 2.420 | 4.71E−08 | 9-11C9 | 1.498 | 7.65E−08 |
| 9-9D5 | 2.353 | 6.16E−10 | 9-11C10 | 2.850 | 7.47E−10 |
| 9-9D9 | 1.116 | 1.44E−09 | 9-11D9 | 2.371 | 2.90E−09 |
| 9-9E1 | 1.525 | 3.31E−08 | 9-11D10 | 0.974 | n/a |
| 9-11E1 | 1.944 | 3.62E−08 | 9-12G10 | 2.853 | 4.91E−09 |
| 9-11E3 | 1.256 | n/a | 9-12G12 | 2.801 | 2.19E−09 |
| 9-11E6 | 3.191 | 1.44E−09 | 9-12H1 | 2.833 | 1.23E−10 |
| 9-11E7 | 2.049 | 5.46E−08 | 9-12H4 | 3.180 | 3.44E−10 |
| 9-11E12 | 2.874 | n/a | 9-12H6 | 3.120 | 5.25E−10 |
| 9-11G2 | 0.696 | n/a | 9-12H9 | 0.424 | n/a |
| 9-11G3 | 0.610 | 1.98E−08 | 9-12H10 | 0.410 | n/a |
| 9-11G5 | 2.728 | 1.81E−09 | 9-13A5 | 2.660 | 3.01E−09 |
| 9-11G9 | 2.688 | n/a | 9-13A9 | 0.664 | n/a |
| 9-11G12 | 3.000 | 1.68E−10 | 9-13B2 | 2.716 | n/a |
| 9-11H1 | 2.900 | 7.30E−10 | 9-13B6 | 2.707 | n/a |
| 9-11H4 | 0.795 | 9.55E−09 | 9-13C3 | 1.674 | 3.85E−10 |
| 9-12B1 | 3.178 | 7.85E−10 | 9-13C8 | 2.247 | n/a |
| 9-12B3 | 3.088 | 7.85E−10 | 9-13C10 | 1.537 | 1.48E−09 |
| 9-12B8 | 1.381 | n/a | 9-13D3 | 2.393 | n/a |
| 9-12B10 | 2.134 | 1.83E−08 | 9-13D4 | 2.221 | 7.64E−09 |
| 9-12C1 | 3.207 | 6.60E−10 | 9-13D8 | 0.949 | n/a |
| 9-12C3 | 2.341 | 1.35E−09 | 9-13E10 | 1.070 | 1.52E−09 |
| 9-12C7 | 2.904 | 7.86E−09 | 9-13E11 | 2.292 | 4.91E−08 |
| 9-12C12 | 2.616 | 7.54E−11 | 9-13F3 | 0.861 | n/a |
| 9-12D5 | 1.980 | 3.12E−09 | 9-13F5 | 0.991 | 9.08E−09 |
| 9-12D10 | 3.138 | 8.69E−11 | 9-13F11 | 2.716 | 9.08E−09 |
| 9-12D11 | 3.180 | 5.83E−11 | 9-13F12 | 1.874 | 2.30E−08 |
| 9-12D12 | 1.791 | 1.50E−08 | 9-13G3 | 2.651 | 1.43E−09 |
| 9-12E1 | 2.383 | 3.84E−11 | 9-13G7 | 1.796 | 2.00E−09 |
| 9-12E6 | 2.716 | 3.85E−11 | 9-13G10 | 1.954 | 5.58E−09 |
| 9-12E12 | 2.835 | 1.16E−09 | 9-14A2 | 3.187 | 2.55E−10 |
| 9-12F1 | 1.970 | 1.29E−08 | 9-14A5 | 2.848 | 9.94E−10 |
| 9-12F3 | 0.457 | n/a | 9-14A9 | 0.597 | 3.24E−08 |
| 9-12F8 | 1.811 | n/a | 9-14C1 | 2.117 | n/a |
| 9-12F10 | 0.690 | n/a | 9-14C4 | 2.992 | n/a |
| 9-12G4 | 2.779 | 6.33E−09 | 9-14C11 | 2.201 | n/a |
| 9-12G5 | 1.417 | n/a | 9-14D1 | 1.741 | n/a |
| 9-12G6 | 2.615 | 1.39E−09 | 9-14D2 | 2.099 | 1.14E−08 |
| 9-12G7 | 1.922 | 1.63E−08 | 9-14D3 | 1.167 | 4.93E−09 |
| 9-14D4 | 1.590 | 1.2E−08 | 9-15F3 | 2.155 | 1.56E−08 |
| 9-14D10 | 1.262 | n/a | 9-15F7 | 2.715 | 4.93E−11 |
| 9-14D12 | 3.280 | 1.4E−11 | 9-15F10 | 0.875 | n/a |
| 9-14E1 | 3.085 | 6.0E−11 | 9-15F12 | 3.353 | 2.32E−09 |
| 9-14E2 | 2.227 | 1.6E−10 | 9-15G1 | 3.419 | 1.75E−09 |
| 9-14E3 | 2.850 | 2.6E−08 | 9-15G10 | 3.372 | 3.60E−10 |
| 9-14E6 | 0.516 | 4.3E−09 | 9-15H4 | 0.657 | n/a |
| 9-14E7 | 2.727 | 5.4E−11 | 9-15H5 | 3.122 | 1.41E−09 |
| 9-14E9 | 2.899 | 2.1E−10 | 9-15H6 | 3.232 | n/a |
| 9-14F1 | 2.696 | 1.7E−09 | 9-16A8 | 2.840 | 1.64E−08 |
| 9-14F3 | 1.383 | 3.0E−09 | 9-16A9 | 2.996 | 1.70E−09 |
| 9-14F5 | 1.354 | n/a | 9-16A11 | 3.254 | 1.04E−09 |
| 9-14F10 | 2.340 | 9.7E−09 | 9-16A12 | 1.645 | 1.19E−08 |
| 9-14F11 | 1.296 | 2.4E−08 | 9-16B2 | 3.089 | 7.83E−11 |
| 9-14F12 | 0.724 | 8.5E−09 | 9-16B10 | 0.971 | 7.16E−09 |
| 9-14G2 | 2.953 | 1.9E−09 | 9-16C4 | 1.913 | n/a |
| 9-14G5 | 2.566 | 1.9E−08 | 9-16C5 | 0.759 | n/a |
| 9-14H6 | 2.172 | 2.7E−09 | LN 3-A5 | 0.929 | 8.30E−12 |
| 9-15A1 | 3.250 | 1.2E−09 | LN 1-C2 | 0.752 | 1.70E−10 |
| 9-15A2 | 2.986 | 3.1E−09 | LN 3-C4 | 0.855 | 6.40E−12 |
| 9-15A6 | 2.313 | n/a | LN 1-G6 | 0.627 | 2.80E−10 |
| 9-15B6 | 3.088 | 1.7E−09 | LN 2-D2 | 0.758 | 8.30E−12 |
| 9-15B8 | 2.135 | 7.1E−08 | LN 2-D6 | 0.784 | 4.50E−11 |
| 9-15C5 | 2.691 | n/a | LN 5-A5 | 1.024 | 1.20E−10 |
| 9-15C6 | 0.470 | n/a | LN 8-A5 | 1.011 | 8.00E−12 |
| 9-15C7 | 2.922 | 6.8E−10 | LN 5-B7 | 1.172 | 4.70E−11 |
| 9-15D8 | 1.519 | 3.6E−11 | LN 5-F12 | 0.881 | 1.50E−10 |
| 9-15D10 | 3.027 | 3.5E−09 | LN 8-G3 | 0.795 | 6.90E−11 |
| 9-15E4 | 3.118 | 9.6E−10 | LN 5-D2 | 0.679 | 3.00E−11 |
| 9-15E8 | 3.127 | 7.1E−10 | LN 6-D3 | 0.754 | 5.30E−11 |
| 9-15E9 | 0.730 | n/a | LN 5-D8 | 0.698 | 7.80E−11 |
| 9-15E11 | 2.582 | 3.7E−06 | SP-14-1B10 | 1.764 | 7.70E−10 |
| 9-15F1 | 3.213 | 1.1E−09 | SP-14- | 1.819 | 7.77E−11 |
| | | | 1H6 | | |
| 9-15F2 | 2.310 | n/a | SP-14- | 0.997 | n/a |
| | | | 2A5 | | |
| B-EF-7- | 2.326 | n/a | SP-14- | 1.342 | n/a |
| 1 A4 | | | 2C7 | | |
| B-EF-7- | 1.275 | n/a | SP-14- | 1.743 | 1.43E−09 |
| 1 A10 | | | 2H4 | | |
| B-EF-7- | 1.009 | n/a | SP-14- | 0.857 | n/a |
| 1C1 | | | 3B5 | | |
| B-EF-7- | 2.155 | n/a | SP-14- | 1.107 | n/a |
| 1C4 | | | 3D5 | | |
| B-EF-7- | 2.011 | n/a | SP-14- | 1.033 | n/a |
| 1C9 | | | 3D8 | | |
| B-EF-7- | 0.808 | n/a | SP-14- | 1.441 | n/a |
| 1D7 | | | 3D12 | | |
| B-EF-7- | 1.124 | n/a | SP-14- | 1.552 | 1.92E−09 |
| 1E2 | | | 4F7 | | |
| B-EF-7- | 0.611 | n/a | SP-14- | 0.520 | n/a |
| 1E4 | | | 4H10 | | |
| B-EF-7- | 1.978 | n/a | SP-14- | 1.096 | n/a |
| 1F5 | | | 5B10 | | |
| B-EF-7- | 2.225 | n/a | SP-14- | 1.637 | 4.26E−11 |
| 1G5 | | | 5C2 | | |
| B-EF-7- | 2.035 | n/a | SP-14- | 1.732 | 1.26E−11 |
| 1G11 | | | 5F9 | | |
| B-EF-7- | 0.912 | n/a | SP-14- | 0.854 | 8.93E−09 |
| 1G12 | | | 5G8 | | |
| B-EF-7- | 0.501 | n/a | SP-14- | 1.648 | 6.78E−10 |
| 1H4 | | | 5H2 | | |
| B-EF-7- | 1.031 | n/a | SP-14- | 1.445 | n/a |
| 1H6 | | | 6C10 | | |
| B-EF-7- | 2.169 | n/a | SP-14- | 1.829 | n/a |
| 2A6 | | | 7E12 | | |
| B-3- | 0.890 | 2.05E−09 | LN-mix- | 1.662 | n/a |
| 1C12 | | | 1C9 | | |
| B-3-1D7 | 0.812 | 2.85E−08 | LN-mix- | 1.394 | n/a |
| | | | 2A2 | | |
| B-3-1 E4 | 1.228 | 2.06E−09 | LN-mix- | 1.298 | n/a |
| | | | 3A2 | | |
| B-3-2C2 | 1.421 | 3.56E−10 | LN-mix- | 1.208 | n/a |
| | | | 4C6 | | |
| B-3-2C5 | 0.874 | 1.86E−09 | LN-mix- | 1.358 | n/a |
| | | | 5B7 | | |
| B-3-2C8 | 1.032 | 3.24E−08 | LN-mix- | 1.539 | n/a |
| | | | 6B11 | | |
| B-3-2D9 | 1.225 | 1.30E−09 | LN-mix- | 0.846 | n/a |
| | | | 7C5 | | |
| B-3-2E5 | 1.047 | 5.92E−09 | LN-mix- | 0.534 | n/a |
| | | | 8D6 | | |
| B-3-2H4 | 0.812 | 4.98E−08 | B-BM- | 0.826 | n/a |
| | | | MIX-11E1 | | |
| B-3- | 1.372 | 2.06E−09 | B-7-2A7 | 1.328 | n/a |
| 3B11 | | | | | |
| B-3-3D8 | 0.785 | 1.36E−09 | B-7-2A8 | 1.519 | 7.22E−10 |
| B-3-3F9 | 1.090 | 5.73E−10 | B-7-2A10 | 1.709 | n/a |
| B-7-1A1 | 1.576 | n/a | B-7-2B7 | 1.803 | 1.38E−10 |
| B-7-1A5 | 1.683 | n/a | B-7-2B11 | 1.099 | n/a |
| B-7-1A8 | 0.531 | n/a | B-7-2C5 | 1.009 | 1.43E−08 |
| B-7- | 1.988 | n/a | B-7-2C10 | 1.669 | n/a |
| 1A10 | | | | | |
| B-7- | 0.655 | n/a | B-7-2C12 | 1.730 | n/a |
| 1B10 | | | | | |
| B-7-1C2 | 1.017 | 8.69E−09 | B-7-2D3 | 1.566 | n/a |
| B-7-1C4 | 1.045 | 2.86E−09 | B-7-2D7 | 1.491 | n/a |
| B-7-1C6 | 1.657 | 8.71E−10 | B-7-2D11 | 0.916 | n/a |
| B-7- | 0.901 | 2.77E−09 | B-7-2E4 | 1.788 | n/a |
| 1C10 | | | | | |
| B-7-1D2 | 1.165 | 1.64E−09 | B-7-2E7 | 1.639 | 1.16E−11 |
| B-7-1D9 | 1.118 | n/a | B-7-2F2 | 1.620 | 1.06E−09 |
| B-7- | 1.720 | 1.86E−10 | B-7-2F3 | 1.582 | 3.17E−09 |
| 1D11 | | | | | |
| B-7-1 E1 | 0.627 | 2.52E−09 | B-7-2F6 | 1.721 | n/a |
| B-7-1 E3 | 1.115 | n/a | B-7-2F10 | 1.748 | 7.90E−12 |
| B-7-1 E6 | 1.794 | 4.65E−11 | B-7-2F11 | 0.867 | n/a |
| B-7-1F4 | 0.694 | n/a | B-7-2G8 | 1.586 | 1.76E−09 |
| B-7-1F7 | 0.673 | 2.48E−09 | B-7-2G9 | 1.553 | n/a |
| B-7-1G4 | 1.673 | 4.05E−11 | B-7-2H10 | 1.641 | 1.44E−11 |
| B-7-1G5 | 1.709 | 2.70E−11 | B-7-3A12 | 0.521 | n/a |
| B-7-1G804 | 1.579 | 4.02E−10 | B-7-3B2 | 1.768 | n/a |
| B-7-1H5 | 1.867 | n/a | B-7-3B7 | 1.032 | 3.61E−08 |
| B-7-2A5 | 1.309 | 4.62E−09 | B-7-3B11 | 0.692 | n/a |
| B-7-2A6 | 0.764 | n/a | B-7-3C7 | 0.795 | n/a |
| | | | B-7-3C10 | 0.676 | n/a |
| | | | B-7-3C11 | 0.633 | 2.43E−08 |
| | | | B-7-3C12 | 1.686 | n/a |
| | | | B-7-3D2 | 0.738 | n/a |
| | | | B-7-3D3 | 1.008 | n/a |
| | | | B-7-3D11 | 1.664 | 1.09E−10 |
| | | | B-7-3F4 | 1.327 | n/a |
| | | | B-7-3F5 | 1.251 | 2.76E−10 |
| | | | B-7-3F9 | 1.532 | n/a |

The hybridomas were further characterised for their affinity for RBD and capacity to compete with the binding of RBD and ACE2. In particular, several assays were carried out; they are illustrated in the paragraphs below.

### Measurement of Affinity

The affinity (K_{D}) for RBD and the kon/koff values were determined by surface plasmon resonance (SPR) using the Carterra LSA instrument. The antibodies showed a range of affinity from pM to nM and the results have been summarised in Table 1 and FIG. 2. A biosensor chip HC200M (Carterra) was bound covalently with an anti-mouse-Fc (Jackson ImmunoResearch #115-005-071) and then treated with the antibodies at different concentrations. The samples were prepared for capture in 384-well plates by diluting them 5 times in HBSTE buffer (final volume 220 ul). The RBD antigen was provided in a purified recombinant monomeric form and was prepared in an HBSTE+BSA 0.5 mg/ml buffer at a starting concentration of 1 μM. At this point the analytes were dispensed into the flow cell in increasing concentrations, allowing for an association time of 5 minutes and a dissociation time of 15 minutes.

### Evaluation of Competition for ACE2 Receptor by Surface Plasmon Resonance

In order to evaluate the capacity of the clones to block the binding of ACE2 to RBD, 500 nM of ACE2 was injected after the last injection of RBD. The recorded binding signals make it possible to determine whether the clones compete with ACE2 for the same binding region on RBD. No additional signals were observed for the selected clones, thus showing that these antibodies share the same ACE2 binding site and could potentially be used as a virus neutralising agent. Table 2 lists the hybridomas that produce antibodies which are competitive or non-competitive for human ACE2.

TABLE 2

| Hybridoma | Competition with |
|---|---|
| Name | ACE2 |
| 9-8G10 | No |
| 2-5F8 | Yes |
| 5-2D11 | Yes |
| 9-8H7 | No |
| 2-8E3 | Yes |
| 9-10G1 | Yes |
| 9-4F4 | Yes |
| 9-7A4 | Yes |
| 5-13D3 | Yes |
| 2-14F3 | Yes |
| 2-15F7 | Yes |
| 9-8F2 | Yes |
| 2-6G4 | Yes |
| 9-5A10 | Yes |
| 5-4H6 | Yes |
| 1-3B8 | Yes |
| 1-3H10 | Yes |
| 1-2C12 | Yes |
| 9-3H9 | Yes |
| 5-7B11 | Yes |
| 9-12G6 | Yes |
| 2-8D6 | No |
| 5-13E12 | Yes |
| 2-16D1 | Yes |
| 2-12C7 | Yes |
| 9-6F11 | Yes |
| 2-10B12 | Yes |
| 9-5B4 | Yes |
| 9-10E8 | No |
| 9-2B12 | Yes |
| 2-6D5 | Yes |
| 5-5F8 | Yes |
| 5-5D10 | Yes |
| 2-12D6 | Yes |
| 5-1B9 | Yes |
| 5-11H5 | Yes |
| 9-7F8 | Yes |
| 5-12B5 | Yes |
| 5-2B12 | Yes |
| 9-13C10 | Yes |
| 5-10C4 | Yes |
| 9-15B6 | Yes |
| 2-5B4 | Yes |
| 5-1A4 | Yes |
| 2-12E1 | Yes |
| 9-15C7 | No |
| 9-6C1 | Yes |
| 2-15B2 | Yes |
| 2-14G2 | Yes |
| 2-11D2 | Yes |
| 5-2F12 | Yes |
| 5-1D5 | Yes |
| 9-10G4 | Yes |
| 5-10F3 | Yes |
| 5-13F5 | Yes |
| 9-6G2 | Yes |
| B-7-1G8 | Yes |
| B-7-1C6 | Yes |
| 5-9C10 | Yes |

### Pseudovirus Neutralisation Test

Following the measurement of affinity by high-throughput SPR, a selection was made of about 430 antibodies with affinity≤1 nM, which were subjected to a first pseudovirus neutralisation assay using three supernatant concentrations (1:2, 1:20, 1:200); the results, normalised for the concentration of immunoglobulins (FIG. 4), are summarised in FIG. 3. One may observe a dose-dependent response for the majority of the tested antibodies. Among the 430 antibodies tested, 59 were selected; the EC50 was calculated for the latter, again using the pseudovirus neutralisation assay. The results are summarised in Table 3 (EC50 values of supernatants tested by means of the pseudovirus assay). As may be observed from the table, the activity of the antibodies covers an EC50 range that goes from about 260 ng/ml to less than 1 ng/ml.

TABLE 3

| | EC50 |
|---|---|
| Antibody | (μg/ml) |
| 9-8G10 | 0.0006 |
| 2-5F8 | 0.0028 |
| 5-2D11 | 0.0030 |
| 9-8H7 | 0.0033 |
| 2-8E3 | 0.0047 |
| 9-10G1 | 0.0056 |
| 9-4F4 | 0.0072 |
| 9-7A4 | 0.0073 |
| 5-13D3 | 0.0115 |
| 2-14F3 | 0.0122 |
| 2-15F7 | 0.0125 |
| 9-8F2 | 0.0145 |
| 2-6G4 | 0.0149 |
| 9-5A10 | 0.0174 |
| 5-4H6 | 0.0175 |
| 1-3B8 | 0.0181 |
| 1-3H10 | 0.0185 |
| 1-2C12 | 0.0206 |
| 9-3H9 | 0.0222 |
| 5-7B11 | 0.0227 |
| 9-12G6 | 0.0232 |
| 2-8D6 | 0.0247 |
| 5-13E12 | 0.0263 |
| 2-16D1 | 0.0272 |
| 2-12C7 | 0.0285 |
| 9-6F11 | 0.0297 |
| 2-10B12 | 0.0326 |
| 9-5B4 | 0.0340 |
| 9-10E8 | 0.0342 |
| 9-2H7 | 0.293 |
| 9-2B12 | 0.0371 |
| 2-6D5 | 0.0379 |
| 5-5F8 | 0.0416 |
| 5-5D10 | 0.0445 |
| 2-12D6 | 0.0454 |
| 5-1B9 | 0.0521 |
| 5-11H5 | 0.0551 |
| 9-7F8 | 0.0558 |
| 5-12B5 | 0.0568 |
| 5-2B12 | 0.0572 |
| 9-13C10 | 0.0592 |
| 5-10C4 | 0.0648 |
| 9-15B6 | 0.0671 |
| 2-5B4 | 0.0707 |
| 5-1A4 | 0.0721 |
| 2-12E1 | 0.0724 |
| 9-15C7 | 0.0741 |
| 9-6C1 | 0.0747 |
| 2-15B2 | 0.0757 |
| 2-14G2 | 0.0798 |
| 2-11D2 | 0.1040 |
| 5-2F12 | 0.1064 |
| 5-1D5 | 0.1153 |
| 9-10G4 | 0.1434 |
| 5-10F3 | 0.1457 |
| 5-13F5 | 0.1693 |
| 9-6G2 | 0.1727 |
| B-7-1G8 | 0.2316 |
| B-7-1C6 | 0.2545 |
| 5-9C10 | 0.2584 |

### Evaluation of Competition by ELISA and Flow Cytometry

The antibodies (59) for which the EC50 was calculated via the pseudovirus inhibition assay were further characterised for their capacity to compete for binding between ACE2 and RBD by means of the ELISA technique (FIG. 5), using recombinant ACE2 and RBD proteins, and by flow cytometry, using the ACE2 receptor expressed in Vero cells and recombinant RBD protein. As expected, most of the antibodies showed to be competitive for binding between RBD and ACE2.

### Production of Monoclonal Mouse Antibodies

The 59 selected hybridomas were then subcloned and mouse antibodies were produced from the subclones and purified. The mouse antibodies of the most active clones were analysed for their capacity to neutralise the SARS-CoV-2 virus in the variants D614G and N501 Y (Table 4). Table 4 shows the EC50 values for the virus variants D614G and N501 Y.

TABLE 4

| | EC50 virus D614G | EC50 virus N501Y |
|---|---|---|
| Antibody | (ng/ml) | (ng/ml) |
| 2-5F8-D7 | 4.53 | 4.60 |
| 2-8E3-B10 | 3.04 | 4.38 |
| 9-7A4-C2 | 3.21 | 4.06 |
| 5-13D3-A10 | 13.02 | 5.95 |
| 9-8F2-B11 | 5.49 | 0.87 |
| 2-6G4-H5 | 19.18 | 7.05 |
| 1-3B8-C7 | 6.14 | 5.74 |
| 1-3H10-C7 | 27.66 | 9.58 |
| 1-2C12-A10 | 6.41 | 2.31 |
| 2-12C7-B3 | 156.4 | N.A |
| 9-10E8-D7 | >1000 | N.A |
| 5-5F8-D3 | 130.0 | N.A |
| 2-12D6-G6 | 13.96 | 17.17 |
| 5-11H5-E3 | 7.00 | 5.73 |
| 5-12B5-D5 | 6.10 | 10.35 |
| 2-5B4-C9 | 305.0 | N.A |
| 5-2F12-C9 | 11.50 | 4.22 |
| B-7-2F10-D11 | >1000 | N.A |
| 5B7-B3 | >1000 | >1000 |
| 9-2H7-D7 | 10.15 | 15.25 |
| SP14-5F9-C9 | <1000 | N.A |

Neutralisation Assay with SARS-CoV-2 Virus Variants D614G and N501 Y

In order to verify whether the mAbs could neutralise the infectivity of the SARS-CoV-2 virus, a neutralisation test was performed. Vero cells (VERO 0100801008 [Vero 76, clone E6, Vero E6] (ATCC@ CRL-1586™) (10,000 cells/well) were seeded 24 hours before infection in a 96-well plate (Costar). On the day of infection, the cells were washed twice. The monoclonal antibodies were incubated at 56° C. for 30 minutes and then diluted twice in cell culture medium. The antibodies, at the initial concentration of 1000 ng/ml, were added to the cell culture medium containing 100 viral particles of SARS-CoV-2 variant D614G or N501Y (B.1.1.7) on a 96-well plate and incubated at 37° C. for 30 minutes in 5% vol/vol CO₂. The mixture of viral antibodies was then added to the cells in 96-well plates and the plates were incubated at 37° C. with a microscopic examination to evaluate the cytopathic effect after 3 days of incubation. The highest dilution of the antibody that showed an effect of inhibiting SARS-CoV-2 was recorded as the neutralising antibody titre. The tests were performed in duplicate with the variants D614G and N501Y. These tests were performed in a BSL-3 facility. The results obtained provided extremely good EC50 values for a limited set of antibodies, with EC50 values that range from micromolar to nanomolar (Table 4). Based on the results obtained, the most potent antibodies were identified (EC50<50 ng/ml), and they were further characterised by means of:
1) affinity assay by BLI for the spike protein and RBD variants. In view of its potentialities, 3-12B12-F4 was tested on a wider spectrum of variants (Table 5 and Table 6);
2) flow cytometry assay to evaluate the binding of purified antibodies to the original spike protein and with VOC mutations in the RBD region (Table 7 and FIG. 7);
3) ELISA to evaluate the binding to the spike protein (Table 8) and to variants of the RBD (Table 9);
4) epitope binning experiments by BLI to select the antibodies that recognise different portions of the RBD region.

In particular, Tables 5 and 6 show measurements of the affinity of the purified antibodies with respect to the RBD variants, Table 7 shows the percentage of binding of the purified antibodies to the spike protein as determined by FACS, Table 8 shows the IC50 value of the purified antibodies tested against the spike protein and Table 9 shows the IC50 value (μg/mi) for the purified antibodies tested for the RBD variants.

TABLE 5

| | | | | | K_{D} RBD |
|---|---|---|---|---|---|
| | | K_{D} RBD | K_{D} RBD | K_{D} RBD | South |
| | K_{D} | British | Australian | Brazilian | African |
| Purified | RBD | variant | variant | variant | variant |
| antibody | WT (M) | (M) | (M) | (M) | (M) |
| 2-8E3-B10 | 2.46E−09 | 2.33E−09 | NO | 1.14E−09 | 3.01E−09 |
| 9-7A4-C2 | 6.43E−10 | <1.0E−12 | 1.53E−06 | 1.62E−09 | 4.51 E−09 |
| 9-8F2-B11 | 9.13E−10 | 5.00E−10 | 5.88E−10 | 1.31E−09 | 5.52E−09 |
| 1-3B8-C7 | 8.18E−10 | 1.21E−09 | NO | 4.83E−10 | 1.38E−09 |
| 2-5F8-D7 | 2.42E−09 | 2.08E−09 | NO | 1.08E−09 | 3.01E−09 |
| 1-2C12-A10 | 4.11E−10 | <1.0E−12 | >1 mM | 1.16E−10 | 4.35E−10 |
| 5-12B5-D5 | 5.24E−10 | 2.01E−09 | 6.84E−08 | 9.85E−11 | <1.0E−12 |
| 2-12C7-B3 | 5.44E−10 | 5.06E−10 | 1.93E−10 | NO | NO |
| 5-11H5-E3 | 9.65E−10 | 3.87E−10 | NO | 3.37E−10 | 9.60E−10 |
| 5-2F12-C9 | 9.85E−11 | 1.19E−10 | NO | 6.14E−11 | <1.0E−12 |
| 9-2H7-D7 | 5.15E−11 | 4.04E−10 | 3.91E−09 | <1.0E−12 | <1.0E−12 |
| 5-13D3-A10 | 2.99E−10 | 5.21E−10 | NO | 5.68E−10 | 9.00E−10 |
| 2-6G4-H5 | 2.56E−09 | 3.43E−09 | NO | 5.68E−10 | 9.00E−10 |
| 2-12D6-G6 | 3.43E−09 | 7.09E−09 | NO | 5.68E−10 | 9.00E−10 |
| 1-3H10-C7 | 9.52E−10 | 9.99E−10 | NO | 5.68E−10 | 9.00E−10 |
| 9-8H7-B4 | 5.43E−09 | 6.57E−09 | 2.78E−09 | 2.23E−09 | 4.09E−09 |
| 9-3H9-3E9-B12 | 5.42E−08 | 6.54E−08 | 8.81E−09 | 1.13E−09 | 2.20E−09 |
| 5-2B12-3E12 | 9.10E−10 | 1.34E−09 | NO | 4.75E−10 | 7.36E−10 |

TABLE 6

| | 3-12B12-F4 |
|---|---|
| RBD Variant | K_{D} (M) |
| WT | 5.76E−11 |
| Omicron BA.1 | 1.21E−09 |
| British | 1.67E−10 |
| Delta | 2.25E−09 |
| South African | 6.49E−10 |
| Australian | 5.76E−11 |
| Brazilian | 1.67E−10 |
| Californian | 2.25E−09 |
| Indian | 1.21E−09 |
| N436K | 6.49E−10 |

TABLE 7

| Antibody | % binding to spike |
|---|---|
| 2-5F8-D7 | 34.94% |
| 2-8E3-B10 | 39.13% |
| 9-7 A4-C2 | 45.53% |
| 5-13D3-A10 | 45.12% |
| 9-8F2-B11 | 90.90% |
| 2-6G4-H5 | 38.51% |
| 1-3B8-C7 | 46.99% |
| 1-3H10-C7 | 40.97% |
| 1-2C12-A10 | 44.99% |
| 2-12C7-B3 | 28.47% |
| 9-10E8-D7 | 51.00% |
| 2-12D6-G6 | 53.37% |
| 5-11H5-E3 | 51.97% |
| 5-12B5-D5 | 55.90% |
| 5-2F12-C9 | 44.40% |
| B-7-2F10-D11 | 27.33% |
| 5B7-B3 | 20.44% |
| 9-2H7-D7 | 54.36% |

TABLE 8

| | IC50 vs spike |
|---|---|
| Antibody | (μg/ml) |
| 2-8E3-B10 | 0.0029 |
| 9-7A4-C2 | 0.0027 |
| 9-8F2-B11 | 0.0037 |
| 1-3B8-C7 | 0.0026 |
| 2-5F8-D7 | 0.0026 |
| 1-2C12-A10 | 0.0034 |
| 5-12B5-D5 | 0.0035 |
| 5-11H5-E3 | 0.0036 |
| 5-2F12-C9 | 0.0044 |
| 9-2H7-D7 | 0.0046 |
| 5-13D3-A10 | 0.0048 |
| 2-6G4-H5 | 0.0046 |
| 2-12D6-G6 | 0.0001 |
| 5-5F8-D3 | 0.0001 |
| 2-12C7-B3 | 0.0058 |

TABLE 9

| | | IC50 British | IC50 Australian | IC50 Brazilian | IC50 South | IC50 Californian | IC50 variant |
|---|---|---|---|---|---|---|---|
| Antibody | IC50 RBD WT | variant | variant | variant | African variant | variant | N439K |
| 2-8E3- | 0.0105 | 0.0114 | 0.0183 | 0.0489 | 0.0153 | 0.0114 | 0.0077 |
| B10 | | | | | | | |
| 9-7A4- | 0.0133 | 0.0126 | 1.0990 | 0.0560 | 0.0132 | 0.0126 | 0.0120 |
| C2 | | | | | | | |
| 9-8F2- | 0.0137 | 0.0156 | 0.0183 | 0.0144 | 0.0090 | 0.0156 | 0.0163 |
| B11 | | | | | | | |
| 1-3B8- | 0.0227 | 0.0255 | 1.2060 | 0.0552 | 0.0226 | 0.0255 | 0.0107 |
| C7 | | | | | | | |
| 2-5F8- | 0.0124 | 0.0146 | 1.0900 | 0.0665 | 0.0099 | 0.0146 | 0.0221 |
| D7 | | | | | | | |
| 1-2C12- | 0.0212 | 0.0311 | >3 μg/ml | 0.0268 | 0.0214 | 0.0311 | 0.0135 |
| A10 | | | | | | | |
| 5-12B5- | 0.0207 | 0.0372 | 1.0800 | 0.0205 | 0.0205 | 0.0372 | 0.0217 |
| D5 | | | | | | | |
| 5-11H5- | 0.0166 | 0.0257 | 0.7923 | 0.0126 | 0.0126 | 0.0257 | 0.0245 |
| E3 | | | | | | | |
| 5-2F12- | 0.0157 | 0.0353 | 1.0560 | 0.0346 | 0.0346 | 0.0353 | 0.0162 |
| C9 | | | | | | | |
| 9-2H7- | 0.0269 | 0.0555 | 0.7923 | 0.0195 | 0.0195 | 0.0555 | 0.0202 |
| D7 | | | | | | | |
| 5-13D3- | 0.0192 | 0.0301 | 0.0844 | 0.0262 | 0.0262 | 0.0301 | 0.0142 |
| A10 | | | | | | | |
| 2-6G4- | 0.0226 | 0.0249 | 0.1211 | 0.0245 | 0.0245 | 0.0249 | 0.0318 |
| H5 | | | | | | | |
| 2-12D6- | 0.1832 | 0.5187 | 0.0183 | 0.1222 | 0.1222 | 0.5187 | 0.0947 |
| G6 | | | | | | | |
| 9-8H7- | 0.0099 | 0.0081 | 0.0086 | 0.0081 | 0.0045 | 0.006400 | 0.0084 |
| B4 | | | | | | | |
| 9-3H9- | 0.0226 | 0.0133 | 0.0128 | 0.0096 | 0.0327 | 0.0170 | 0.0241 |
| 3E9- | | | | | | | |
| B12 | | | | | | | |
| 5-2B12- | 0.0053 | 0.0083 | 3.243e−011 | 0.0083 | 0.0077 | 0.0077 | 0.0064 |
| 3E12 | | | | | | | |

The antibodies were initially identified to bind the RBD portion of SARS-COV-2, a site previously defined as substantial for neutralisation of the virus. However, during the genetic vaccination, as explained previously, the animals were immunised using the DNA that codes for the entire spike protein in order to be able to obtain antibodies that recognised the RBD in the context of the complete protein. For this reason, the capacity of the selected monoclonal antibodies to bind the spike protein was confirmed by ELISA (Table 8) and flow cytometry (Table 7) assays. Similarly, it was interesting to understand whether the binding to the RBD could be compromised by the mutations occurring in this portion of the protein and, therefore, the binding of the monoclonals to several of the RBD variants currently present was evaluated by means of ELISA (Table 9) and flow cytometry (FIG. 7) assays. The results of the ELISA test made it possible to obtain an IC50 value for every antibody. The results of greatest interest, which enabled the binding of the antibodies to their target to be characterised in a more thorough manner, came from the ELISA tests for the RBD variants. By means of this test, it was possible to show that that the antibodies possess a good capacity to bind all the RBD variants with the exception of the Australian variant, which was weakly recognised by most of the antibodies analysed except for four of them:
1) 3-12B12-F4, which recognises with comparable affinity all the RBD variants tested in the assay, including the omicron and Australian variants.
2) 9-8F2-B11, which recognises with comparable affinity all the RBD variants tested in the assay, including the Australian one. 3) 9-2H7-D7, which recognises all the variants well and the Australian variant to a medium degree.
4) 9-74-C2 recognises all the variants well, and similarly the Australian variant to a medium degree.

The analysis was then repeated with affinity assays by BLI (Table 5 and Table 6); the activity of 9-8F2-B11 was further confirmed through a study on a pseudovirus model in vivo (FIG. 16). Furthermore, the antibodies 9-2H7-D7 and 9-74-C2 were identified through in vitro pseudovirus assays as possible candidates to be used as alternatives to 9-8F2-B11 or in combination therewith to increase its effectiveness and obtain better prevention against the outbreak of possible resistant mutants.

### In Vitro Pseudovirus Assay for 3-12B12-F4, 9-8F2-B11, 9-2H7-D7 and 9-7A4-C2.

In addition to the results previously described, a second pseudovirus test was performed which was focused on the three selected purified antibodies 9-8F2-B11, 9-2H7-D7 and 9-7A4-C2; on this occasion, an evaluation was made of their neutralising properties not only vis-à-vis the WT pseudovirus, but also against some of the main variants currently present, namely D614G, B1.1.7 (British), P.1 (Brazilian), B.1.351 (African) and delta, in both their mouse and humanised forms. In this context it was possible to determine that the IC50 values of the tested antibodies differ based on the type of variant tested, showing in particular that 9-8F2-B11 possesses a less effective neutralising power against the African variant and a slightly reduced one versus the Brazilian variant, a lacking that is however absolutely made up for by the other two selected antibodies 9-2H7-D7 and 9-7A4-C2, as may be seen from the values shown in the graph (FIG. 17, FIG. 18 and Table 10). In view of these results, the possibility of using such antibodies in combination as a cocktail formulation appears to be an extremely useful approach and may lead to the total neutralisation of a large set of variants. Despite the excellent results obtained in the assay, the three selected antibodies, in both their mouse and humanised forms, proved ineffective in neutralising the Omicron variant or BA.1. In this regard, it was verified that the antibody 3-12B112-F4 is capable of actively neutralising the Omicron BA.1 variant, with neutralisation values that are extremely high also compared to the commercial antibody Sotrovimab (Table 11). The antibody was also subcloned to obtain the antibody 3-12B12-F4, which confirms the values obtained in the assay by the parent clone. Given the interest towards the potentialities of this antibody, it was further characterised to evaluate its affinity towards the RBD variants and showed an excellent affinity for each of them (Table 6).

TABLE 10

| | WT | British | Brazilian | African | Delta |
|---|---|---|---|---|---|
| | D614G | B1.1.7 | P.1 | B1.1.351 | B1.617.2 |
| | IC50 (ng/ml) | | | | |
| 9-8F2-B11 | 11 | 7.7 | 57 | 175 | 127 |
| mouse | | | | | |
| 9-8F2-B11 | 16 | 13 | 131 | 111 | 50 |
| chimera | | | | | |
| 9-8F2-B11 | 16 | 11 | 3700 | 12000 | 113 |
| humanised | | | | | |
| 9-2H7-D7 | 35 | 40 | 63 | 46 | 107 |
| mouse | | | | | |
| 9-2H7-D7 | 19 | 11 | 11 | 21 | 22 |
| chimera | | | | | |
| 9-2H7-D7 | 29 | 16 | 45 | 65 | 36 |
| humanised | | | | | |
| 9-7A4-C2 | 16 | 18 | 9 | 20 | 52 |
| mouse | | | | | |
| 9-7 A4-C2 | 19 | 18 | 6 | 30 | 63 |
| chimera | | | | | |
| 9-7 A4-C2 | 27 | 15 | 10 | 42 | 73 |
| humanised | | | | | |

TABLE 11
% inhibition of Omicron BA.1

| | 10 ug/ml | 5 ug/ml | 2.5 ug/ml | 1 ug/ml |
|---|---|---|---|---|
| 3-12B12 | 99.4% | 99.2% | 98.0% | 97.2% |
| 3-12B12-F4 | 99.0% | | | |
| Sotrovimab | | | | 61.0% |

### In Vivo Pseudovirus Assay for 9-8F2-B11

The analysis of effectiveness was conducted in K18-hACE2 mice (B6.Cg-Tg(K18-ACE2)2Primn/J catalogue 034860), immunocompetent transgenic mice that express the human form of the ACE2 receptor under the control of the human promoter of keratin 18. On day 0 the mice were anaesthetised by inhalation of isofluorane and infected intranasally with 10 ml/nostril of SARS-CoV-2-Spike (D614G)-Luc pseudovirus or a Lentivirus-Luc used as a negative control of the infection. 2 hours before and 4 hours after the infection, the animals were treated with 10 ml/nostril of anti-SARS-CoV-2 monoclonal antibody 9-8F2-B11 at a concentration of 1.78 mg/ml or with PBS. The clinical signs of the animals were evaluated over the course of the study, including body weight, which was recorded daily. Since the pseudovirus carries the gene for luciferase, it was possible to monitor the infection by acquiring the bioluminescence signal produced.

96 hours after infection, all the animals were analysed alive (FIG. 16) by means of the Imaging IVIS 200 system in order to quantitatively measure the bioluminescence associated with the presence of the pseudovirus throughout the animal's body. After the in vivo analysis, the animals were sacrificed by asphyxia and the nasal turbinates were removed from them and analysed; the largest presence of a signal was observed there in the absence of treatment, whereas in the mice treated with the antibody 9-8F2-B11 the signal was completely inhibited (FIG. 16).

### Bio-Layer Interferometry Measurement of Antibody Affinity for RBD Variants.

Binding studies were conducted using the Octet Red system (ForteBio). All steps were carried out at 25° C. with shaking at 600 rpm in a 96-well plate (96-well microplate, black, 655209, Greiner bio-one) containing 200 μl of solution in each well. The 1× kinetic buffer (cat. N. 18-1105, Forte Bio) was used in this study for the dilution of antibodies and analytes and for washing the sensors. The kinetic assays were performed by first capturing the mAbs using Octet anti-mouse Fc biosensors (anti-mouse IgG Fc Capture Biosensors, cat. No. 18-5088, FORTEBIO). The biosensors were immersed for 10 minutes in the 1× buffer; this was followed by a 60-second measurement of the base signal. At this point the mouse monoclonal antibodies (10 μg mL) were loaded for 300 seconds (until the biosensor was completely saturated). After a step of washing in 1× kinetic buffer for 120 s, the biosensor tips conjugated with the mAbs were immersed for 300 seconds in wells containing different antigen concentrations (RBD 6×HIS) to evaluate the association curve; then followed 900 seconds of dissociation in the kinetic buffer. The data of the binding curve were collected and then analysed using the data analysis software v11.1 (FORTEBIO). The binding sensorgrams were aligned at the start of the antigen binding cycle and after the single reference subtraction. The Kd values were calculated using a Langmuir global binding model 1:1. The biosensor tips with the mAbs were also immersed in wells containing kinetic buffer to allow the single reference subtractions to compensate for the natural dissociations of the captured mAbs. The biosensor tips were used without regeneration (Table 5).

Flow Cytometry Assay to Evaluate the Binding of the Purified Antibodies to the Original Spike Protein and with Mutations of the VOCs in the RBD Region

Briefly, HEK-293 cells (catalogue no. R70007) were transfected with an empty vector (EV) as a negative control or pNebAd6 SARS-CoV-2 spike-FL or variants. 200,000 cells/well (96-well plate) were treated with purified antibodies at a final concentration of 1 μg/ml for 20′ at 4° C. The cells were then washed in FACS buffer and stained with anti-mouse IgG Alexa Fluor 488-conjugated secondary antibodies or anti-human IgG Alexa Fluor 488-conjugated secondary antibodies for 20′ at 4° C. Finally, the cells were washed in FACS buffer and resuspended in 1% formaldehyde-PBS before being run on a CytoFlex flow cytometer (Beckman Coulter). The analysis was performed using CytExpert software (Beckman Coulter) (FIG. 7, Table 7).

### ELISA Assay to Evaluate Binding to the Spike Protein and to Variants of the RBD Domain

The plates were coated with 50 μl per well of the full-length spike protein or RBD-6His WT (D614), N501Y (British variant), N439K, S477N (Australian variant), South African variant, Californian variant and Brazilian variant diluted in PBS to a final concentration of 1 μg/ml. The coating step was carried out overnight at a temperature of 4 degrees. The plates were subsequently washed three times with PBS/T and then blocked with 100 ul of blocking solution (3% BSA) for a period of one hour at room temperature. Then followed a step of incubation with the antigen, in which the purified antibodies at the starting concentration of 3 μg/ml in 1% BSA with 1:3 serial dilutions were incubated overnight at 4 degrees. The plates were washed three times with PBS/T and were subsequently incubated with anti-mouse IgG Human AP secondary antibody (Southern Biotech) at the final dilution of 1:4000. The plates were subsequently developed with a development reagent at room temperature for 1 h; then followed a reading at a wavelength of 405 nm and a calculation of the IC50 (Tables 7 and 8).

The hybridomas were subjected to sequencing of the variable regions of the light and heavy chains.

The sequences of the VH and VL regions of the mouse antibodies 3-12B12-F4, 9-8F2-B11, 9-2H7-D7, and 97A4-C2 are the following:
3-12B12-F4—VH: SEQ ID NO:51;
3-12B12-F4—VH: SEQ ID NO:52;
9-8F2-B11—VH1: SEQ ID NO:1;
9-8F2-B11—VL1: SEQ ID NO:2;
9-7A4-C2—VH: SEQ ID NO:3;
9-7A4-C2—VL: SEQ ID NO:4;
9-2H7-D7—VH1: SEQ ID NO:5;
9-2H7-D7—VL1: SEQ ID NO:6;

The information obtained from sequencing was used to generate chimeric antibodies (FIGS. 8, 9, 12 and 14) and the sequences of the antibodies 9-8F2-B11, 9-7A4-C2 and 9-2H7-D7 were humanised in order to obtain a completely human antibody (FIGS. 10, 11, 13 and 15).

The VH and VL sequences of the humanised antibodies 9-8F2-B11, 9-7A4-C2 and 9-2H7-D7 are shown below:
9-8F2-B11—humVH3: SEQ ID NO:12;
9-8F2-B11—humVL3: SEQ ID NO:13.
9-7A4-C2—VH: SEQ ID NO:14;
9-7A4-C2—VL: SEQ ID NO:15;
9-2H7-D7—VH1: SEQ ID NO:16;
9-2H7-D7—VL1: SEQ ID NO:17

The humanised antibody 9-8F2-B11 obtained maintains the same characteristics of affinity for the RBD and the VOCs (Table 12) as the mouse antibody and can thus be effectively used in the treatment and prevention of COVID-19.

Table 12 shows the measurement of affinity of purified humanised 9-8F2-B11 VH3VL3 for RBD variants.

TABLE 12

| | | | | | KD RBD |
|---|---|---|---|---|---|
| | | KD RBD | KD RBD | KD RBD | South |
| | | British | Australian | Brazilian | African |
| | KD RBD | variant | variant | variant | variant |
| Antibody | WT (M) | (M) | (M) | (M) | (M) |
| 9-8F2-B11 | 2.26E−09 | 1.40E−09 | 2.76E−09 | 6.07E−09 | 1.82E−08 |
| VH3VL3 | | | | | |

By western blot analysis it was possible to confirm that some VH and VL combinations, both in the case of the chimeric antibody and in the case of the humanised antibody, were more productive and more greatly expressed than others, an essential requirement for their production on a large scale and subsequent clinical use. These results show what VH and VL combinations are the most productive, as they maintain the ability to recognise the RBD according to the ELISA tests performed downstream of purification to determine the affinity for the RBD as the main target.

### Preparation of Samples for Sequencing

For the preparation of samples for the purpose of the subsequent sequencing, messenger RNA was initially extracted (Qiagen) and cDNA was synthesised (Superscript 3 Thermo). With the aim of rendering possible the amplification of the variable regions of the heavy and light chains (VH and VL), specific primers for mouse IgG were used. Subsequently, the sequences of the fragments obtained of the expected height were sequenced by NGS with the Illumina platform. As regards the antibody 9-8F2-B11, three equivalent sequences were obtained for the variable region of the heavy chain and two equivalent sequences for the variable region of the light chain, whose combinations, produced as a chimeric antibody—i.e. made up of the variable regions of the mouse heavy and light chains and the constant regions of the human heavy chain of the IgG1 subclass and the constant region of the human light chain of the kappa subclass—maintain the capacity to bind the RBD (FIG. 9).

### Production and Purification of Chimeric and Humanised Antibodies

Chimeric and humanised antibodies were produced by transient transfection of ExpiCHO high density cells (ExpiCHO™ Expression System Kit, A29133) with ExpiFectamine 293 cationic lipid transfection reagent (Thermo Fisher) according to the manufacturer's instructions. The supernatant containing the proteins was collected after a week and subjected to clarification by centrifugation and filtration for the subsequent purification steps with protein A as explained in the previous paragraph. The purity of the antibodies was evaluated by SDS-PAGE and western blot analysis, conducted under both reduced and non-reduced conditions and with standard methods (FIGS. 8, 10, 12, 13, 14 and 15) and their capacity to bind the recombinant RBD was evaluated by means of the ELISA technique (FIGS. 9, 11, 12, 13, 14 and 15).

The humanised sequences of the variable regions of the heavy chain and light chains of the antibodies 9-8F2-B11, 9-2H7-D7 and 9-7A4-C2 capable of binding the RBD are respectively indicated as the nucleotide sequences SEQ ID NO:29 and 30, SEQ ID NO:33 and 34, SEQ ID NO:31 and 32 (whilst the corresponding amino acid sequences are indicated as SEQ ID NO:12 and 13, SEQ ID NO:16 and 17, SEQ ID NO:14 and 15), which were cloned in an expression vector containing the constant region of the heavy chain of subclass IgG1 and the constant region of the light chain of subclass kappa, but they could also be expressed as IgG2, IgG3 or IgG4 for the heavy chain and as lambda for the light chain.

In particular, the humanised antibody containing the variable regions of the heavy chain with SEQ ID:29 and of the light chain with SEQ ID:30 maintains the same characteristics of affinity for the RBD and VOCs (Table 12) as the mouse antibody and can thus be effectively used in the treatment and prevention of COVID-19 through administration by the intravenous, subcutaneous, intramuscular, or intranasal route by nebulisation.

### REFERENCES

(1) Mahase, E. Covid-19: WHO declares pandemic because of “alarming levels” of spread, severity, and inaction. BMJ 368, m1036 (2020).
(2) Corti, D., Misasi, J., Mulangu, S., Stanley, D. A., Kanekiyo, M., Wollen, S., Ploquin, A., Doria-Rose, N. A., Staupe, R. P., Bailey, M., et al. (2016). Protective monotherapy against lethal Ebola virus infection by a potently neutralizing antibody. Science 351, 1339-1342.
(3) Wang, L., Shi, W., Chappell, J. D., Joyce, M. G., Zhang, Y., Kanekiyo, M., Becker, M. M., van Doremalen, N., Fischer, R., Wang, N., et al. (2018). Importance of Neutralizing Monoclonal Antibodies Targeting Multiple Antigenic Sites on the Middle East Respiratory Syndrome Coronavirus Spike Glycoprotein To Avoid Neutralization Escape. J. Virol. 92, 1-21. Wang, C., Li, W., Drabek, D., Okba, N. M. A., Haperen, R. van, Osterhaus, A. D. M. E., Kuppeveld, F. J. M. van, Haagmans, B. L., Grosveld, F., and Bosch, B.-J. (2020). A human monoclonal antibody blocking SARS-CoV-2 infection. Nat. Commun. Published online May 4, 2020. https://doi.org/10.1038/s41467-020-16256-y.
(4) Scheid, J. F., Mouquet, H., Feldhahn, N., Seaman, M. S., Velinzon, K., Pietzsch, J., Ott, R. G., Anthony, R. M., Zebroski, H., Hurley, A., et al. (2009). Broad diversity of neutralizing antibodies isolated from memory B cells in HIV-infected individuals. Nature 458, 636-640. Seifert, M., and Ku″ ppers, R. (2016). Human memory B cells. Leukemia 30, 2283-2292.
(5) Ou, X. et al. Characterization of spike glycoprotein of SARS-CoV-2 on virus entry and its immune cross-reactivity with SARS-CoV. Nat. Commun. 11,1620 (2020).
(6) Song W, Gui M, Wang X, Xiang Y. Cryo-EM structure of the SARS coronavirus spike glycoprotein in complex with its host cell receptor ACE2. PLoS Pathog. 2018 Aug. 13; 14(8): e1007236. doi: 10.1371/journal.ppat.1007236. eCollection 2018 Aug.
(7) J. Mair-Jenkins et al., J. Infect. Dis. 211, 80-90 (2015).
(8) J. H. Ko et al., Antivir. Ther. 23, 617-622 (2018).
(9) C. Shen et al., JAMA 323, 1582 (2020).
(10) S. Mulangu et al., N. Engl. J. Med. 381, 2293-2303 (2019).
(11) J. van Griensven et al., N. Engl. J. Med. 374, 33-42 (2016).
(12) Baum A, Ajithdoss D, Copin R, et al. REGN-COV2 antibodies prevent and treat SARS-CoV-2 infection in rhesus macaques and hamsters. Science 2020 Oct. 9.
(13) Shuo D, Yunlong C, Qinyu Z et al., Structurally resolved SARS-CoV-2 antibody shows high efficacy in severely infected hamsters and provides a potent cocktail pairing strategy. Cell 2020
(14) Wang S., Yu P., Rongjuab W., Shasha J., Min W., Chao S. et al., (2020) Characterization of neutralizing antibody with prophylactic and therapeutic efficacy against SARS-CoV-2 in rhesus monkeys. Nat. commun.11,5752
(15) Zost S., Gilchuk P., Case J., Binshtein E. et al., Potently neutralizing and protective human antibodies against SARS-Cov-2. https://doi.org/10.1038/s41586-020-2548-6.
(16) Du S., Cao Y., Zhu Q. et al., Structurally resolved SARS-CoV-2 antibody shows high efficacy in several infected hamsters and provides a potent cocktail pairing strategy. Cell 183,1-11. https://doi.org/10.1016/j.cell.2020.09.035.
(17) Kreye J., Reincke S M., Kornau H C. et al., A therapeutic non-self-reactive SARS-Cov-2 antibody protects from lung pathology in a COVID-19 hamster model. Cell 183, 1-12, November 2020. https://doi.org/10.1016/j.cell.2020.09.049.
(18) Li W., Chen C., Drelich A., Martinez D R. et al., Rapid identification of a human antibody with high prophylactic and therapeutic efficacy in three animal models of SARS-CoV-2 infection. https://doi.org/10.1073/pnas.2010197117.
(19) Lifei Yang, Weihan Liu, Xin Yu, Meng Wu, Janice M Reichert, Mitchell Ho. COVID-19 Antibody Therapeutics Tracker: A Global Online Database of Antibody Therapeutics for the Prevention and Treatment of COVID-19. Antibody Therapeutics, tbaa020, https://doi.org/10.1093/abt/tbaa020.
(20) Jones B E, Brown-Augsburger P L, Corbett K S, Westendorf K, Davies J, Cujec T P, Wiethoff C M, Blackbourne J L, Heinz B A, Foster D, Higgs R E, Balasubramaniam D, Wang L, Bidshahri R, Kraft L, Hwang Y, Zentelis S, Jepson K R, Goya R, Smith M A, Collins D W, Hinshaw S J, Tycho S A, Pellacani D, Xiang P, Muthuraman K, Sobhanifar S, Piper M H, Triana F J, Hendle J, Pustilnik A, Adams A C, Berens S J, Baric R S, Martinez D R, Cross R W, Geisbert T W, Borisevich V, Abiona O, Belli H M, de Vries M, Mohamed A, Dittmann M, Samanovic M, Mulligan M J, Goldsmith J A, Hsieh C L, Johnson N V, Wrapp D, McLellan J S, Barnhart B C, Graham B S, Mascola J R, Hansen C L, Falconer E: LY-CoV555, a rapidly isolated potent neutralizing antibody, provides protection in a non-human primate model of SARS-CoV-2 infection. bioRxiv. 2020 Oct. 1. doi: 10.1101/2020.09.30.318972.
(21) FDA Emergency Use Authorization Fact Sheet: Bamlanivimab [https://www.fda.gov/media/143603/download].
(22) Hansen, J., Baum, A., Pascal, K. E., Russo, V., Giordano, S., Wloga, E., Fulton, B. O., Yan, Y., Koon, K., Patel, K. et al. (2020) Studies in humanized mice and convalescent humans yield a SARS-CoV-2 antibody cocktail. eabd0827.
(23) Baum, A., Fulton, B. O., Wloga, E., Copin, R., Pascal, K. E., Russo, V., Giordano, S., Lanza, K., Negron, N., Ni, M. et al. (2020) Antibody cocktail to SARS-CoV-2 spike protein prevents rapid mutational escape seen with individual antibodies. eabd0831.
(24) Pinto D, Park Y J, Beltramello M et al., Cross-neutralization of SARS-CoV-2 by a human monoclonal SARS-CoV antibody. Nature volume 583, pages290-295(2020)
(25) Zost S J et al. Potently neutralizing human antibodies that block SARS-CoV-2 receptor binding and protect animals. Nature. 2020. DOI: 10.1038/s41586-020-2548-6
(26) Atal, S. and Fatima, Z. (2020) IL-6 Inhibitors in the Treatment of Serious COVID-19: A Promising Therapy? Pharmaceut Med, 1-9.
(27) Luo, P., Liu, Y., Qiu, L., Liu, X., Liu, D. and Li, J. (2020) Tocilizumab treatment in COVID-19: A single center experience. J Med Virol, 92, 814-818.
(28) Xu D, Alegre M L, Varga S S, Rothermel A L, Collins A M, Pulito V L, Hanna L S, Dolan K P, Parren P W, Bluestone J A, Jolliffe L K, Zivin R A. In vitro characterization of five humanized OKT3 effector function variant antibodies. Cell Immunol. (2000) 200:16-26
(29) Shields R L, Namenuk A K, Hong K, Meng Y G, Rae J, Briggs J, Xie D, Lai J, Stadlen A, Li B, Fox J A, Presta L G. High resolution mapping of the binding site on human IgG1 for Fc gamma RI, Fc gamma RII, Fc gamma RIII, and FcRn and design of IgG1 variants with improved binding to the Fc gamma R. J. Biol. Chem. 2001, 276:6591-604
(30) Moore G L, Chen H, Karki S, Lazar G A. Engineered Fc variant antibodies with enhanced ability to recruit complement and mediate effector functions. MAbs. 2010 Mar-Apr;2(2):181-9
(31) Petkova S B, Akilesh S, Sproule T J, Christianson G J, Al Khabbaz H, Brown A C, Presta L G, Meng Y G, Roopenian D C. Enhanced half-life of genetically engineered human IgG1 antibodies in a humanized FcRn mouse model: potential application in humorally mediated autoimmune disease. Int Immunol. 2006 December; 18(12):1759-69.
(32) Fattori E, the Monica N, Ciliberto G, Toniatti C. Electro-gene-transfer: A new approach for muscle gene delivery. Somat Cell Mol Genet. 2002;27(1-6):75-83.

1. A humanized antibody obtained from a mouse antibody, or a mouse antibody, said mouse antibody being selected from the group consisting of:
an antibody (3-12B12-F4) comprising
the variable region of the heavy chain (VH) EVQLQQSGAELVRPGALVKL SCKASGFNIKDYYMHWVKQRPEQGLEWIGWIDPENGN SIYDPKFQGKASITADTSFNTVNLHL SSLTSEDTAVYYCAPYYYDSTYVGTMDYWGQG TSVTVSS (SEQ ID NO:51), and
the variable region of the light chain (VL) QIVLTQSPAIMSASPGEKVTMTCSASSSVGYMYWYQQKPGSSPRLLIYDTSNLASGVPV RFGGSGSGTSYSLTISRMEAEDAATYYCQQWSSYPPRTFGGGTKLEIK (SEQ ID NO:52);
an antibody (9-8F2-B11) comprising
the variable region of the heavy chain (VH) EVQLQQSGAELVKPGASVKLSCTASGFNIKETYVHWVKQRPEQGLEWIGRIDPAIGDS EYDPKFQGKATVTADTSSNTAYLQLSRLTSEDTAVYYCARTWGPFFDFWGQGTTLTVS S (SEQ ID NO:1), and
the variable region of the light chain (VL) DVVMTQTPLSLPVSLGDQASISCRSSQSLVHSHGNTFLHWYLQKPGQSPKVLIYKVSSR FSGVPDRFSGSGAGTDFTLKISRVEAEDLGVYFCSQSTHVPYTFGGGTKLEIK (SEQ ID NO:2);
an antibody (9-7A4-C2) comprising
the variable region of the heavy chain (VH)
EVQLQQSGPELVKPGASVKISCKTSGYTFTEYTMYWVKQSHGKSLEWIGGINPN NGDSSYIQKFKGKATLTVDESSSTAYMELRSLTSEDSAVYYCARDGYPNSYAMDYWG QGTSVTVSS (SEQ ID NO:3) and
the variable region of the light chain (VL)
DIVMSQSPSSLAVSVGDKVTMSCKSSQSLLYSTNQKNYLAWYQQKPGQSPKLLI YWASTRESGVPDRFTGSGSGTDFTLTIGSVRPEDLAVYYCQQYYSYPWTFGGGTKLEIK (SEQ ID NO:4); and
an antibody (9-2H7-D7) comprising
the variable region of the heavy chain (VH)
EVQLQQSGPELVKPGASVTISCKTSGYTFTEYTMYWVKQSHGKSLEWIGGINPY IGDTSYNQNFKGKATLTVDKSSSTAYMELRSLTSEDSAVYYCARDGYPDYSAMDFWG QGTSVTVSS (SEQ ID NO:5) and
the variable region of the light chain (VL) DIVMSQSPSSLAVSVGEKVTMNCKSSQNLLYSSNQKNYLAWYQQKPGQSPKLLIYWAS TRESGVPDRFTGSGSGTDFTLTISSVKAEDLAVYSCQQYYTYPWTFGGGTKLEIK (SEQ ID NO:6).

2. The humanized antibody according to claim 1, wherein said humanized antibody comprises
a VH region comprising a first CDR region GFNIKETY (SEQ ID NO:7), a second CDR region IDPAIGDS (SEQ ID NO:8), and a third CDR region of sequence ARTWGPFFDF (SEQ ID NO:9), and
a VL region comprising a first CDR region QSLVHSHGNTF (SEQ ID NO:10), a second CDR region of sequence KVS, and a third CDR region of sequence SQSTHVPYT (SEQ ID NO:11);
a VH region comprising a first CDR region of sequence GYTFTEYT (SEQ ID NO:42), a second CDR region of sequence INPNNGDS (SEQ ID NO:43), and a third CDR region of sequence ARDGYPNSYAMDY (SEQ ID NO:44), and
a VL region comprising a first CDR region of sequence QSLLYSTNQKNY (SEQ ID NO:45), a second CDR region of sequence WAS, and a third CDR region of sequence QQYYSYPWT (SEQ ID NO:46); or
a VH region comprising a first CDR region of sequence GYTFTEYT (SEQ ID NO:42), a second CDR region of sequence INPYIGDT (SEQ ID NO:47), and a third CDR region of sequence ARDGYPDYSAMDF (SEQ ID NO:48), and
a VL region comprising a first CDR region of sequence QNLLYSSNNKNY (SEQ ID NO:49), a second CDR region of sequence WAS, and a third CDR region of sequence QQYYTYPWT (SEQ ID NO:50).

3. The humanized antibody according to claim 1, wherein said humanized antibody is selected from the group consisting of:
an antibody (humanised 9-8F2-B11) comprising
the VH region
QVQLVQSGAEVKKPGASVKVSCKASGFNIKETYVHWVRQAPGQGLEWMGRID PAIGDSEYAQKFQGRVTMTRDTSISTAYMELSRLRSDDTAVYYCARTWGPFFDFWGQ GTLVTVSS (SEQ ID NO:12); and
the VL region
DIVMTQTPLSLSVTPGQPASISCKSSQSLLHSHGNTFLHWYLQKPGQSPQVLIYK VSSRFSGVPDRFSGSGSGTDFTLKISRVEAEDVGVYYCSQSTHVPYTFGQGTKLEIK (SEQ ID NO:13);
an antibody (humanised 9-7A4-C2) comprising
the VH region
EVQLQQSGAEVKKPGASVKVSCKTSGYTFTEYTMYWVRQAPGQRLEWMGGIN PNNGDSSYIQKFQGRVTITRDTSASTAYMELSSLRSEDTAVYYCARDGYPNSYAMDYW GQGTLVTVSS (SEQ ID NO:14) and
the VL region
DIVMTQSPDSLAVSLGDRATINCKSSQSLLYSTNQKNYLAWYQQKPGQPPKLLI YWASTRESGVPDRFSGSGSGTDFTLTISSLQAEDVAVYYCQQYYSYPWTFGQGTKLEIK (SEQ ID NO:15); or
an antibody (humanised 9-2H7-D7) comprising
the VH region
QVQLQQSGAEVKKPGASVKISCKTSGYTFTEYTMYWVKQSHGKSLEWMGGIN PYIGDTSYNQNFKGRVTMTRDKSTSTAYMELSSLTSEDSAVYYCARDGYPDYSAMDF WGQGTSVTVSS (SEQ ID NO:16) and
the VL region
DIVMTQSPSSLAVSVGERVTINCKSSQNLLYSSNNKNYLAWYQQKPGQSPKLLI YWASTRESGVPDRFSGSGSGTDFTLTISSVQAEDVAVYSCQQYYTYPWTFGGGTKLEI K (SEQ ID NO:17).

4. A polynucleotide having a nucleotide sequence encoding an antibody as defined in claim 1.

5. The polynucleotide according to claim 4, wherein
the nucleotide sequence encoding SEQ ID NO:51 is GAGGTGCAGCTGCAGCAGAGCGGCGCCGAGCTGGTGAGGCCCGGCGCCCTGGTGAA GCTGAGCTGCAAGGCCAGCGGCTTCAACATCAAGGACTACTACATGCACTGGGTG AAGCAGAGGCCCGAGCAGGGCCTGGAGTGGATCGGCTGGATCGACCCCGAGAACG GCAACAGCATCTACGACCCCAAGTTCCAGGGCAAGGCCAGCATCACCGCCGACACC AGCTTCAACACCGTGAACCTGCACCTGAGCAGCCTGACCAGCGAGGACACCGCCGT GTACTACTGCGCCCCCTACTACTACGACAGCACCTACGTGGGCACCATGGACTA CTGGGGCCAGGGCACCAGCGTGACCGTGAGCAGC (SEQ ID NO:53);
the nucleotide sequence encoding SEQ ID NO:52 is CAGATCGTGCTGACCCAGAGCCCCGCCATCATGAGCGCCAGCCCCGGCGAGAAGGT GACCATGACCTGCAGCGCCAGCAGCAGCGTGGGCTACATGTACTGGTACCAGCAG AAGCCCGGCAGCAGCCCCAGGCTGCTGATCTACGACACCAGCAACCTGGCCAGCG GCGTGCCCGTGAGGTTCGGCGGCAGCGGCAGCGGCACCAGCTACAGCCTGACCATC AGCAGGATGGAGGCCGAGGACGCCGCCACCTACTACTGCCAGCAGTGGAGCAGCT ACCCCCCCAGGACCTTCGGCGGCGGCACCAAGCTGGAGATCAAG (SEQ ID NO:54);
the nucleotide sequence encoding SEQ ID NO:1 is GAGGTGCAGCTGCAGCAGAGCGGCGCCGAGCTGGTGAAGCCCGGCGCCAGCGTGA AGCTGAGCTGCACCGCCAGCGGCTTCAACATCAAGGAGACCTACGTGCACTGGGT GAAGCAGAGGCCCGAGCAGGGCCTGGAGTGGATCGGCAGGATCGACCCCGCCATC GGCGACAGCGAGTACGACCCCAAGTTCCAGGGCAAGGCCACCGTGACCGCCGACA CCAGCAGCAACACCGCCTACCTGCAGCTGAGCAGGCTGACCAGCGAGGACACCGCC GTGTACTACTGCGCCAGGACCTGGGGCCCCTTCTTCGACTTCTGGGGCCAGGGCA CCACCCTGACCGTGAGCAGC (SEQ ID NO:18);
the nucleotide sequence encoding SEQ ID NO:2 is GACGTGGTGATGACCCAGACCCCCCTGAGCCTGCCCGTGAGCCTGGGCGACCAGGC CAGCATCAGCTGCAGGAGCAGCCAGAGCCTGGTGCACAGCCACGGCAACACCTT CCTGCACTGGTACCTGCAGAAGCCCGGCCAGAGCCCCAAGGTGCTGATCTACAAGG TGAGCAGCAGGTTCAGCGGCGTGCCCGACAGGTTCAGCGGCAGCGGCGCCGGCACC GACTTCACCCTGAAGATCAGCAGGGTGGAGGCCGAGGACCTGGGCGTGTACTTCTG CAGCCAGAGCACCCACGTGCCCTACACCTTCGGCGGCGGCACCAAGCTGGAGATC AAG (SEQ ID NO:19);
the nucleotide sequence encoding SEQ ID NO:3 is
GAGGTGCAGCTGCAGCAGTCTGGACCAGAGCTGGTGAAGCCTGGAGCATCCG TGAAGATCTCTTGCAAGACCAGCGGCTATACCTTCACAGAGTACACAATGTATTG GGTGAAGCAGTCCCACGGCAAGTCTCTGGAGTGGATCGGCGGCATCAACCCAAACA ATGGCGATAGCTCCTATATCCAGAAGTTTAAGGGCAAGGCCACCCTGACAGTGGAC GAGTCTAGCTCCACCGCCTACATGGAGCTGCGGTCCCTGACATCTGAGGACAGCGCC GTGTACTATTGTGCCAGAGATGGCTACCCCAATAGCTATGCCATGGACTACTGGG GCCAGGGCACCTCCGTGACAGTGTCTAGC (SEQ ID NO:20) and
the nucleotide sequence encoding SEQ ID NO:4 is
GACATCGTGATGTCCCAGTCTCCTAGCTCCCTGGCCGTGAGCGTGGGCGATA AGGTGACCATGTCCTGCAAGTCTAGCCAGTCTCTGCTGTACAGCACAAACCAGAA GAATTACCTGGCCTGGTATCAGCAGAAGCCCGGCCAGAGCCCTAAGCTGCTGATCT ATTGGGCATCCACCAGGGAGTCTGGAGTGCCAGACAGATTCACCGGCAGCGGCTCC GGAACAGACTTCACCCTGACAATCGGCTCTGTGCGGCCAGAGGACCTGGCCGTGTA CTATTGTCAGCAGTACTATTCCTACCCCTGGACCTTCGGCGGCGGCACAAAGCTG GAGATCAAG (SEQ ID NO:21);
the nucleotide sequence encoding SEQ ID NO:5 is
GAAGTGCAACTACAACAAAGTGGTCCAGAACTGGTGAAGCCTGGCGCTTCTG TGACCATCTCCTGCAAGACCTCTGGCTACACCTTTACCGAGTACACCATGTACTGG GTCAAGCAGTCTCACGGAAAATCTCTGGAGTGGATCGGCGGCATCAACCCCTACAT CGGCGACACCTCCTACAACCAGAACTTCAAGGGCAAGGCCACCCTGACCGTGGAC AAGTCCTCCTCTACCGCCTACATGGAACTGCGGAGCCTGACATCCGAGGATTCTGCT GTGTACTACTGTGCCAGAGATGGATATCCTGACTACTCCGCCATGGACTTCTGGG GCCAGGGCACCTCCGTGACAGTGTCCAGC (SEQ ID NO:22) and
the nucleotide sequence encoding SEQ ID NO:6 is
GATATCGTGATGAGTCAAAGTCCAAGTAGTCTCGCCGTGTCCGTGGGAGAGA AGGTGACCATGAACTGCAAGTCCTCTCAGAATCTGCTGTACTCCTCCAACCAGAA GAACTACCTGGCCTGGTACCAGCAAAAACCTGGCCAGTCTCCTAAGCTGCTGATCT ACTGGGCTTCTACCAGAGAGTCTGGCGTGCCCGATCGGTTTACCGGCTCCGGCTCT GGCACCGACTTCACACTGACCATCTCCAGCGTCAAGGCCGAGGACCTGGCTGTGTAC AGCTGTCAGCAGTACTACACCTATCCTTGGACCTTCGGCGGCGGAACAAAGCTGG AAATCAAG (SEQ ID NO:23).

6-7. (canceled)

8. An expression vector comprising a nucleotide sequence as defined in claim 4.

9. The vector according to claim 8, wherein said vector is selected from the group consisting of a plasmid, for example bacterial plasmids, an RNA, an RNA that replicates, amplicons obtained by PCR, a viral vector.

10. A cell comprising an expression vector as defined in claim 8.

11. A pharmaceutical composition comprising one or more humanised antibodies as defined in claim 1.

12. The pharmaceutical composition according to claim 11, wherein said one or more antibodies consist of the following alternative combinations:
a combination of four antibodies, wherein a first humanised antibody 3-12B12-F4 is obtained from the mouse antibody having the VH sequence SEQ ID NO:51 and the VL sequence SEQ ID NO:52, the second humanised antibody 9-8F2-B11 comprises the VH sequence SEQ ID NO:12 and the VL sequence SEQ ID NO:13, the third humanised antibody 9-7A4-C2 comprises the VH sequence SEQ ID NO:14 and the VL sequence SEQ ID NO:15, and the fourth humanised antibody 9-2H7-D7 comprises the VH sequence SEQ ID NO:16 and the VL sequence SEQ ID NO:17;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-8F2-B11 comprises the VH sequence SEQ ID NO:12 and the VL sequence SEQ ID NO:13, and a third humanised antibody 9-7A4-C2 comprises the VH sequence SEQ ID NO:14 and the VL sequence SEQ ID NO:15;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-8F2-B11 comprises the VH sequence SEQ ID NO:12 and the VL sequence SEQ ID NO:13, and a third humanised antibody 9-2H7-D7 comprises the VH sequence SEQ ID NO:16 and the VL sequence SEQ ID NO:17;
a combination of three antibodies, wherein a first humanised antibody 3-12B12-F4 is obtained from the mouse antibody having the VH sequence SEQ ID NO:51 and the VL sequence SEQ ID NO:52, a second humanised antibody 9-7A4-C2 comprises the VH sequence SEQ ID NO:14 and the VL sequence SEQ ID NO:15, and a third humanised antibody 9-2H7-D7 comprises the VH sequence SEQ ID NO:16 and the VL sequence SEQ ID NO:17;
a combination of three antibodies, wherein a first humanised antibody 9-8F2-B11 comprises the VH sequence SEQ ID NO:12 and the VL sequence SEQ ID NO:13, a second humanised antibody 9-7A4-C2 comprises the VH sequence SEQ ID NO:14 and the VL sequence SEQ ID NO:15, and a third humanised antibody 9-2H7-D7 comprises the VH sequence SEQ ID NO:16 and the VL sequence SEQ ID NO:17;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-8F2-B11 comprises the VH sequence SEQ ID NO:12 and the VL sequence SEQ ID NO:13;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-7A4-C2 comprises the VH sequence SEQ ID NO:14 and the VL sequence SEQ ID NO:15;
a combination of two antibodies, wherein a first humanised antibody 3-12B12-F4 is obtained from the mouse antibody having the VH sequence SEQ ID NO:51 and the VL sequence SEQ ID NO:52, and a second humanised antibody 9-2H7-D7 comprises the VH sequence SEQ ID NO:16 and the VL sequence SEQ ID NO:17;
a combination of two antibodies, wherein a first humanised antibody 9-8F2-B11 comprises the VH sequence SEQ ID NO:12 and the VL sequence SEQ ID NO:13, and a second humanised antibody 9-7A4-C2 comprises the VH sequence SEQ ID NO:14 and the VL sequence SEQ ID NO:15;
a combination of two antibodies, wherein a first humanised antibody 9-8F2-B11 comprises the VH sequence SEQ ID NO:12 and the VL sequence SEQ ID NO:13, and a second humanised antibody 9-2H7-D7 comprises the VH sequence SEQ ID NO:16 and the VL sequence SEQ ID NO:17;
a combination of two antibodies, wherein a first humanised antibody 9-7A4-C2 comprises the VH sequence SEQ ID NO:14 and the VL sequence SEQ ID NO:15, and a second humanised antibody 9-2H7-D7 comprises the VH sequence SEQ ID NO:16 and the VL sequence SEQ ID NO:17.

13-26. (canceled)

27. A polynucleotide having a nucleotide sequence encoding an antibody as defined in claim 2.

28. The polynucleotide sequence according to claim 27, wherein the nucleotide sequence encoding SEQ ID NO:7 is GGCTTCAACATCAAGGAGACCTAC (SEQ ID NO:24), the nucleotide sequence encoding SEQ ID NO:8 is ATCGACCCCGCCATCGGCGACAGC (SEQ ID NO:25), the nucleotide sequence encoding SEQ ID NO:9 is GCCAGGACCTGGGGCCCCTTCTTCGACTTC (SEQ ID NO:26), the nucleotide sequence encoding SEQ ID NO:10 is CAGAGCCTGGTGCACAGCCACGGCAACACCTTC (SEQ ID NO:27), the nucleotide sequence encoding KVS is AAGGTGAGC and the nucleotide sequence encoding SEQ ID NO:11 is AGCCAGAGCACCCACGTGCCCTACACC (SEQ ID NO:28).

29. A polynucleotide having a nucleotide sequence encoding an antibody as defined in claim 3.

30. The polynucleotide sequence according to claim 29, wherein
the nucleotide sequence encoding SEQ ID NO:12 is CAGGTGCAGCTGGTGCAGAGCGGCGCCGAGGTGAAGAAGCCCGGCGCCAGCGTGA AGGTGAGCTGCAAGGCCAGCGGCTTCAACATCAAGGAGACCTACGTGCACTGGGTG AGGCAGGCCCCCGGCCAGGGCCTGGAGTGGATGGGCAGGATCGACCCCGCCATCGG CGACAGCGAGTACGCCCAGAAGTTCCAGGGCAGGGTGACCATGACCAGGGACACCA GCATCAGCACCGCCTACATGGAGCTGAGCAGGCTGAGGAGCGACGACACCGCCGTG TACTACTGCGCCAGGACCTGGGGCCCCTTCTTCGACTTCTGGGGCCAGGGCACCCTG GTGACCGTGAGCAGC (SEQ ID NO:29);
the nucleotide sequence encoding SEQ ID NO:13 is GACATCGTGATGACCCAGACCCCCCTGAGCCTGAGCGTGACCCCCGGCCAGCCCGC CAGCATCAGCTGCAAGAGCAGCCAGAGCCTGCTGCACAGCCACGGCAACACCTTCC TGCACTGGTACCTGCAGAAGCCCGGCCAGAGCCCCCAGGTGCTGATCTACAAGGTG AGCAGCAGGTTCAGCGGCGTGCCCGACAGGTTCAGCGGCAGCGGCAGCGGCACCGA CTTCACCCTGAAGATCAGCAGGGTGGAGGCCGAGGACGTGGGCGTGTACTACTGCA GCCAGAGCACCCACGTGCCCTACACCTTCGGCCAGGGCACCAAGCTGGAGATCAAG (SEQ ID NO:30);
the nucleotide sequence encoding SEQ ID NO:14 is GAGGTGCAGCTGCAGCAGAGCGGCGCCGAGGTGAAGAAGCCCGGCGCCAGCGTGA AGGTGAGCTGCAAGACCAGCGGCTACACCTTCACCGAGTACACCATGTACTGGGTG AGGCAGGCCCCCGGCCAGAGGCTGGAGTGGATGGGCGGCATCAACCCCAACAACG GCGACAGCAGCTACATCCAGAAGTTCCAGGGCAGGGTGACCATCACCAGGGACACC AGCGCCAGCACCGCCTACATGGAGCTGAGCAGCCTGAGGAGCGAGGACACCGCCGT GTACTACTGCGCCAGGGACGGCTACCCCAACAGCTACGCCATGGACTACTGGGGCC AGGGCACCCTGGTGACCGTGAGCAGC (SEQ ID NO:31);
the nucleotide sequence encoding SEQ ID NO:15 is GACATCGTGATGACCCAGAGCCCCGACAGCCTGGCCGTGAGCCTGGGCGACA GGGCCACCATCAACTGCAAGAGCAGCCAGAGCCTGCTGTACAGCACCAACCAGAAG AACTACCTGGCCTGGTACCAGCAGAAGCCCGGCCAGCCCCCCAAGCTGCTGATCTA CTGGGCCAGCACCAGGGAGAGCGGCGTGCCCGACAGGTTCAGCGGCAGCGGCAGC GGCACCGACTTCACCCTGACCATCAGCAGCCTGCAGGCCGAGGACGTGGCCGTGTA CTACTGCCAGCAGTACTACAGCTACCCCTGGACCTTCGGCCAGGGCACCAAGCTGG AGATCAAG (SEQ ID NO:32);
the nucleotide sequence encoding SEQ ID NO:16 is CAGGTGCAGCTGCAGCAGAGCGGCGCCGAGGTGAAGAAGCCCGGCGCCAGCGTGA AGATCAGCTGCAAGACCAGCGGCTACACCTTCACCGAGTACACCATGTACTGGGTG AAGCAGAGCCACGGCAAGAGCCTGGAGTGGATGGGCGGCATCAACCCCTACATCGG CGACACCAGCTACAACCAGAACTTCAAGGGCAGGGTGACCATGACCAGGGACAAG AGCACCAGCACCGCCTACATGGAGCTGAGCAGCCTGACCAGCGAGGACAGCGCCGT GTACTACTGCGCCAGGGACGGCTACCCCGACTACAGCGCCATGGACTTCTGGGGCC AGGGCACCAGCGTGACCGTGAGCAGC (SEQ ID NO:33) and
the nucleotide sequence encoding SEQ ID NO:17 is GACATCGTGATGACCCAGAGCCCCAGCAGCCTGGCCGTGAGCGTGGGCGAGAGGGT GACCATCAACTGCAAGAGCAGCCAGAACCTGCTGTACAGCAGCAACAACAAGAACT ACCTGGCCTGGTACCAGCAGAAGCCCGGCCAGAGCCCCAAGCTGCTGATCTACTGG GCCAGCACCAGGGAGAGCGGCGTGCCCGACAGGTTCAGCGGCAGCGGCAGCGGCA CCGACTTCACCCTGACCATCAGCAGCGTGCAGGCCGAGGACGTGGCCGTGTACAGC TGCCAGCAGTACTACACCTACCCCTGGACCTTCGGCGGCGGCACCAAGCTGGAGAT CAAG (SEQ ID NO:34).

31. A pharmaceutical composition comprising one or more humanised antibodies as defined in claim 2 together with one or more excipients and/or adjuvants.

32. A pharmaceutical composition comprising one or more humanised antibodies as defined in claim 3 together with one or more excipients and/or adjuvants.

33. A pharmaceutical composition comprising one or more nucleotide sequences as defined in claim 4, together with one or more excipients and/or adjuvants.

34. A pharmaceutical composition comprising one or more nucleotide sequences as defined in claim 27, together with one or more excipients and/or adjuvants.

35. A pharmaceutical composition comprising one or more together with one or more or one or more nucleotide sequences as defined in claim 29, together with one or more excipients and/or adjuvants.

36. A pharmaceutical composition comprising one or more vectors as defined in claim 8.

37. A method for reducing likelihood of or treatment of SARS-CoV-2 infection and COVID-19 disease in a subject in need thereof, the method comprising administering one or more humanized antibodies of claim 1 to the subject.

38. A method for reducing likelihood of or treatment of SARS-CoV-2 infection and COVID-19 disease in a subject in need thereof, the method comprising administering one or more humanized antibodies of claim 2 to the subject.

39. A method for reducing likelihood of or treatment of SARS-CoV-2 infection and COVID-19 disease in a subject in need thereof, the method comprising administering one or more humanized antibodies of claim 3 to the subject.

40. A method for reducing likelihood of or treatment of SARS-CoV-2 infection and COVID-19 disease in a subject in need thereof, the method comprising administering the polynucleotide of claim 4 to the subject.

41. A method for reducing likelihood of or treatment of SARS-CoV-2 infection and COVID-19 disease in a subject in need thereof, the method comprising administering the polynucleotide of claim 27 to the subject.

42. A method for reducing likelihood of or treatment of SARS-CoV-2 infection and COVID-19 disease in a subject in need thereof, the method comprising administering the polynucleotide of claim 29 to the subject.

43. A method for reducing likelihood of or treatment of SARS-CoV-2 infection and COVID-19 disease in a subject in need thereof, the method comprising administering the pharmaceutical composition of claim 11 to the subject.

44. A method for reducing likelihood of or treatment of SARS-CoV-2 infection and COVID-19 disease in a subject in need thereof, the method comprising administering the pharmaceutical composition of claim 12 to the subject.

45. A method for reducing likelihood of or treatment of SARS-CoV-2 infection and COVID-19 disease in a subject in need thereof, the method comprising administering the pharmaceutical composition of claim 33 to the subject.

46. A method for reducing likelihood of or treatment of SARS-CoV-2 infection and COVID-19 disease in a subject in need thereof, the method comprising administering the pharmaceutical composition of claim 34 to the subject.

47. A method for reducing likelihood of or treatment of SARS-CoV-2 infection and COVID-19 disease in a subject in need thereof, the method comprising administering the pharmaceutical composition of claim 35 to the subject.

48. An in vitro diagnostic method for the detection of SARS-CoV-2, the method comprising:
obtaining a biological sample from a subject;
exposing said sample to an antibody according to claim 1; and
detecting binding of the antibody to SARS-CoV-2 present in the sample.


SEQUENCE LISTING (54 sequences)

SEQ ID NO: 1 - protein, 117 aa, synthetic construct
  REGION 1..117: note = Heavy chain variable region (VH) of antibody 9-8F2-B11
EVQLQQSGAELVKPGASVKLSCTASGFNIKETYVHWVKQRPEQGLEWIGRIDPAIGDSEYDPKFQGKATVTADTSSNTAYLQLSRLTSEDTAVYYCARTWGPFFDFWGQGTTLTVSS

SEQ ID NO: 2 - protein, 112 aa, synthetic construct
  REGION 1..112: note = Light chain variable region (VL) of antibody 9-8F2-B11
DVVMTQTPLSLPVSLGDQASISCRSSQSLVHSHGNTFLHWYLQKPGQSPKVLIYKVSSRFSGVPDRFSGSGAGTDFTLKISRVEAEDLGVYFCSQSTHVPYTFGGGTKLEIK

SEQ ID NO: 3 - protein, 120 aa, synthetic construct
  REGION 1..120: note = Heavy chain variable region (VH) of antibody 9-7A4-C2
EVQLQQSGPELVKPGASVKISCKTSGYTFTEYTMYWVKQSHGKSLEWIGGINPNNGDSSYIQKFKGKATLTVDESSSTAYMELRSLTSEDSAVYYCARDGYPNSYAMDYWGQGTSVTVSS

SEQ ID NO: 4 - protein, 113 aa, synthetic construct
  REGION 1..113: note = Light chain variable region (VL) of antibody 9-7A4-C2
DIVMSQSPSSLAVSVGDKVTMSCKSSQSLLYSTNQKNYLAWYQQKPGQSPKLLIYWASTRESGVPDRFTGSGSGTDFTLTIGSVRPEDLAVYYCQQYYSYPWTFGGGTKLEIK

SEQ ID NO: 5 - protein, 120 aa, synthetic construct
  REGION 1..120: note = Heavy chain variable region (VH) of antibody 9-2H7-D7
EVQLQQSGPELVKPGASVTISCKTSGYTFTEYTMYWVKQSHGKSLEWIGGINPYIGDTSYNQNFKGKATLTVDKSSSTAYMELRSLTSEDSAVYYCARDGYPDYSAMDFWGQGTSVTVSS

SEQ ID NO: 6 - protein, 113 aa, synthetic construct
  REGION 1..113: note = Light chain variable region (VL) of antibody 9-2H7-D7
DIVMSQSPSSLAVSVGEKVTMNCKSSQNLLYSSNQKNYLAWYQQKPGQSPKLLIYWASTRESGVPDRFTGSGSGTDFTLTISSVKAEDLAVYSCQQYYTYPWTFGGGTKLEIK

SEQ ID NO: 7 - protein, 8 aa, synthetic construct
  REGION 1..8: note = CDR region
GFNIKETY

SEQ ID NO: 8 - protein, 8 aa, synthetic construct
  REGION 1..8: note = CDR region
IDPAIGDS

SEQ ID NO: 9 - protein, 10 aa, synthetic construct
  REGION 1..10: note = CDR region
ARTWGPFFDF

SEQ ID NO: 10 - protein, 11 aa, synthetic construct
  REGION 1..11: note = CDR region
QSLVHSHGNTF

SEQ ID NO: 11 - protein, 9 aa, synthetic construct
  REGION 1..9: note = CDR region
SQSTHVPYT

SEQ ID NO: 12 - protein, 117 aa, synthetic construct
  REGION 1..117: note = Heavy chain variable region (VH) of the humanized antibody9-8F2-B11
QVQLVQSGAEVKKPGASVKVSCKASGFNIKETYVHWVRQAPGQGLEWMGRIDPAIGDSEYAQKFQGRVTMTRDTSISTAYMELSRLRSDDTAVYYCARTWGPFFDFWGQGTLVTVSS

SEQ ID NO: 13 - protein, 112 aa, synthetic construct
  REGION 1..112: note = Light chain variable region (VL) of the humanized antibody9-8F2-B11
DIVMTQTPLSLSVTPGQPASISCKSSQSLLHSHGNTFLHWYLQKPGQSPQVLIYKVSSRFSGVPDRFSGSGSGTDFTLKISRVEAEDVGVYYCSQSTHVPYTFGQGTKLEIK

SEQ ID NO: 14 - protein, 120 aa, synthetic construct
  REGION 1..120: note = Heavy chain variable region (VH) of humanized antibody 9-7A4-C2
EVQLQQSGAEVKKPGASVKVSCKTSGYTFTEYTMYWVRQAPGQRLEWMGGINPNNGDSSYIQKFQGRVTITRDTSASTAYMELSSLRSEDTAVYYCARDGYPNSYAMDYWGQGTLVTVSS

SEQ ID NO: 15 - protein, 113 aa, synthetic construct
  REGION 1..113: note = Light chain variable region (VL) of humanized antibody 9-7A4-C2
DIVMTQSPDSLAVSLGDRATINCKSSQSLLYSTNQKNYLAWYQQKPGQPPKLLIYWASTRESGVPDRFSGSGSGTDFTLTISSLQAEDVAVYYCQQYYSYPWTFGQGTKLEIK

SEQ ID NO: 16 - protein, 120 aa, synthetic construct
  REGION 1..120: note = Heavy chain variable region (VH) of humanized antibody 9-2H7-D7
QVQLQQSGAEVKKPGASVKISCKTSGYTFTEYTMYWVKQSHGKSLEWMGGINPYIGDTSYNQNFKGRVTMTRDKSTSTAYMELSSLTSEDSAVYYCARDGYPDYSAMDFWGQGTSVTVSS

SEQ ID NO: 17 - protein, 113 aa, synthetic construct
  REGION 1..113: note = Light chain variable region (VL) of humanized antibody 9-2H7-D7
DIVMTQSPSSLAVSVGERVTINCKSSQNLLYSSNNKNYLAWYQQKPGQSPKLLIYWASTRESGVPDRFSGSGSGTDFTLTISSVQAEDVAVYSCQQYYTYPWTFGGGTKLEIK

SEQ ID NO: 18 - DNA, 351 bp, synthetic construct
  misc_feature 1..351: note = Nucleotide sequence encoding Heavy chain variable region (VH) of antibody 9-8F2-B11
  source 1..351: mol_type = other DNA
gaggtgcagctgcagcagagcggcgccgagctggtgaagcccggcgccagcgtgaagctgagctgcaccgccagcggcttcaacatcaaggagacctacgtgcactgggtgaagcagaggcccgagcagggcctggagtggatcggcaggatcgaccccgccatcggcgacagcgagtacgaccccaagttccagggcaaggccaccgtgaccgccgacaccagcagcaacaccgcctacctgcagctgagcaggctgaccagcgaggacaccgccgtgtactactgcgccaggacctggggccccttcttcgacttctggggccagggcaccaccctgaccgtgagcagc

SEQ ID NO: 19 - DNA, 336 bp, synthetic construct
  misc_feature 1..336: note = Nucleotide sequence encoding Light chain variable region (VL) of antibody 9-8F2-B11
  source 1..336: mol_type = other DNA
gacgtggtgatgacccagacccccctgagcctgcccgtgagcctgggcgaccaggccagcatcagctgcaggagcagccagagcctggtgcacagccacggcaacaccttcctgcactggtacctgcagaagcccggccagagccccaaggtgctgatctacaaggtgagcagcaggttcagcggcgtgcccgacaggttcagcggcagcggcgccggcaccgacttcaccctgaagatcagcagggtggaggccgaggacctgggcgtgtacttctgcagccagagcacccacgtgccctacaccttcggcggcggcaccaagctggagatcaag

SEQ ID NO: 20 - DNA, 360 bp, synthetic construct
  misc_feature 1..360: note = Nucleotide sequence encoding heavy chain variable region (VH) of antibody 9-7A4-C2
  source 1..360: mol_type = other DNA
gaggtgcagctgcagcagtctggaccagagctggtgaagcctggagcatccgtgaagatctcttgcaagaccagcggctataccttcacagagtacacaatgtattgggtgaagcagtcccacggcaagtctctggagtggatcggcggcatcaacccaaacaatggcgatagctcctatatccagaagtttaagggcaaggccaccctgacagtggacgagtctagctccaccgcctacatggagctgcggtccctgacatctgaggacagcgccgtgtactattgtgccagagatggctaccccaatagctatgccatggactactggggccagggcacctccgtgacagtgtctagc

SEQ ID NO: 21 - DNA, 339 bp, synthetic construct
  misc_feature 1..339: note = Nucleotide sequence encoding light chain variable region (VL) of antibody 9-7A4-C2
  source 1..339: mol_type = other DNA
gacatcgtgatgtcccagtctcctagctccctggccgtgagcgtgggcgataaggtgaccatgtcctgcaagtctagccagtctctgctgtacagcacaaaccagaagaattacctggcctggtatcagcagaagcccggccagagccctaagctgctgatctattgggcatccaccagggagtctggagtgccagacagattcaccggcagcggctccggaacagacttcaccctgacaatcggctctgtgcggccagaggacctggccgtgtactattgtcagcagtactattcctacccctggaccttcggcggcggcacaaagctggagatcaag

SEQ ID NO: 22 - DNA, 360 bp, synthetic construct
  misc_feature 1..360: note = Nucleotide sequence encoding Heavy chain variable region (VH) of antibody 9-2H7-D7
  source 1..360: mol_type = other DNA
gaagtgcaactacaacaaagtggtccagaactggtgaagcctggcgcttctgtgaccatctcctgcaagacctctggctacacctttaccgagtacaccatgtactgggtcaagcagtctcacggaaaatctctggagtggatcggcggcatcaacccctacatcggcgacacctcctacaaccagaacttcaagggcaaggccaccctgaccgtggacaagtcctcctctaccgcctacatggaactgcggagcctgacatccgaggattctgctgtgtactactgtgccagagatggatatcctgactactccgccatggacttctggggccagggcacctccgtgacagtgtccagc

SEQ ID NO: 23 - DNA, 339 bp, synthetic construct
  misc_feature 1..339: note = Nucleotide sequence encoding Light chain variable region (VL) of antibody 9-2H7-D7
  source 1..339: mol_type = other DNA
gatatcgtgatgagtcaaagtccaagtagtctcgccgtgtccgtgggagagaaggtgaccatgaactgcaagtcctctcagaatctgctgtactcctccaaccagaagaactacctggcctggtaccagcaaaaacctggccagtctcctaagctgctgatctactgggcttctaccagagagtctggcgtgcccgatcggtttaccggctccggctctggcaccgacttcacactgaccatctccagcgtcaaggccgaggacctggctgtgtacagctgtcagcagtactacacctatccttggaccttcggcggcggaacaaagctggaaatcaag

SEQ ID NO: 24 - DNA, 24 bp, synthetic construct
  misc_feature 1..24: note = Nucleotide sequence encoding the CDR region SEQ ID NO:7
  source 1..24: mol_type = other DNA
ggcttcaacatcaaggagacctac

SEQ ID NO: 25 - DNA, 24 bp, synthetic construct
  misc_feature 1..24: note = Nucleotide sequence encoding the CDR region SEQ ID NO:8
  source 1..24: mol_type = other DNA
atcgaccccgccatcggcgacagc

SEQ ID NO: 26 - DNA, 30 bp, synthetic construct
  misc_feature 1..30: note = Nucleotide sequence encoding the CDR region SEQ ID NO:9
  source 1..30: mol_type = other DNA
gccaggacctggggccccttcttcgacttc

SEQ ID NO: 27 - DNA, 33 bp, synthetic construct
  misc_feature 1..33: note = Nucleotide sequence encoding the CDR region SEQ ID NO:10
  source 1..33: mol_type = other DNA
cagagcctggtgcacagccacggcaacaccttc

SEQ ID NO: 28 - DNA, 27 bp, synthetic construct
  misc_feature 1..27: note = Nucleotide sequence encoding the CDR region SEQ ID NO:11
  source 1..27: mol_type = other DNA
agccagagcacccacgtgccctacacc

SEQ ID NO: 29 - DNA, 351 bp, synthetic construct
  misc_feature 1..351: note = Nucleotide sequence encoding Heavy chain variable region (VH) ofthe humanized antibody 9-8F2-B11
  source 1..351: mol_type = other DNA
caggtgcagctggtgcagagcggcgccgaggtgaagaagcccggcgccagcgtgaaggtgagctgcaaggccagcggcttcaacatcaaggagacctacgtgcactgggtgaggcaggcccccggccagggcctggagtggatgggcaggatcgaccccgccatcggcgacagcgagtacgcccagaagttccagggcagggtgaccatgaccagggacaccagcatcagcaccgcctacatggagctgagcaggctgaggagcgacgacaccgccgtgtactactgcgccaggacctggggccccttcttcgacttctggggccagggcaccctggtgaccgtgagcagc

SEQ ID NO: 30 - DNA, 336 bp, synthetic construct
  misc_feature 1..336: note = Nucleotide sequence encoding Light chain variable region (VL) of humanized antibody 9-8F2-B11
  source 1..336: mol_type = other DNA
gacatcgtgatgacccagacccccctgagcctgagcgtgacccccggccagcccgccagcatcagctgcaagagcagccagagcctgctgcacagccacggcaacaccttcctgcactggtacctgcagaagcccggccagagcccccaggtgctgatctacaaggtgagcagcaggttcagcggcgtgcccgacaggttcagcggcagcggcagcggcaccgacttcaccctgaagatcagcagggtggaggccgaggacgtgggcgtgtactactgcagccagagcacccacgtgccctacaccttcggccagggcaccaagctggagatcaag

SEQ ID NO: 31 - DNA, 360 bp, synthetic construct
  misc_feature 1..360: note = Nucleotide sequence encoding heavy chain variable region (VH) of humanized antibody 9-7A4-C2
  source 1..360: mol_type = other DNA
gaggtgcagctgcagcagagcggcgccgaggtgaagaagcccggcgccagcgtgaaggtgagctgcaagaccagcggctacaccttcaccgagtacaccatgtactgggtgaggcaggcccccggccagaggctggagtggatgggcggcatcaaccccaacaacggcgacagcagctacatccagaagttccagggcagggtgaccatcaccagggacaccagcgccagcaccgcctacatggagctgagcagcctgaggagcgaggacaccgccgtgtactactgcgccagggacggctaccccaacagctacgccatggactactggggccagggcaccctggtgaccgtgagcagc

SEQ ID NO: 32 - DNA, 339 bp, synthetic construct
  misc_feature 1..339: note = Nucleotide sequence encoding light chain variable region (VL) of humanized antibody 9-7A4-C2
  source 1..339: mol_type = other DNA
gacatcgtgatgacccagagccccgacagcctggccgtgagcctgggcgacagggccaccatcaactgcaagagcagccagagcctgctgtacagcaccaaccagaagaactacctggcctggtaccagcagaagcccggccagccccccaagctgctgatctactgggccagcaccagggagagcggcgtgcccgacaggttcagcggcagcggcagcggcaccgacttcaccctgaccatcagcagcctgcaggccgaggacgtggccgtgtactactgccagcagtactacagctacccctggaccttcggccagggcaccaagctggagatcaag

SEQ ID NO: 33 - DNA, 360 bp, synthetic construct
  misc_feature 1..360: note = Nucleotide sequence encoding heavy chain variable region (VH) of humanized antibody 9-2H7-D7
  source 1..360: mol_type = other DNA
caggtgcagctgcagcagagcggcgccgaggtgaagaagcccggcgccagcgtgaagatcagctgcaagaccagcggctacaccttcaccgagtacaccatgtactgggtgaagcagagccacggcaagagcctggagtggatgggcggcatcaacccctacatcggcgacaccagctacaaccagaacttcaagggcagggtgaccatgaccagggacaagagcaccagcaccgcctacatggagctgagcagcctgaccagcgaggacagcgccgtgtactactgcgccagggacggctaccccgactacagcgccatggacttctggggccagggcaccagcgtgaccgtgagcagc

SEQ ID NO: 34 - DNA, 339 bp, synthetic construct
  misc_feature 1..339: note = Nucleotide sequence encoding light chain variable region (VL) of humanized antibody 9-2H7-D7
  source 1..339: mol_type = other DNA
gacatcgtgatgacccagagccccagcagcctggccgtgagcgtgggcgagagggtgaccatcaactgcaagagcagccagaacctgctgtacagcagcaacaacaagaactacctggcctggtaccagcagaagcccggccagagccccaagctgctgatctactgggccagcaccagggagagcggcgtgcccgacaggttcagcggcagcggcagcggcaccgacttcaccctgaccatcagcagcgtgcaggccgaggacgtggccgtgtacagctgccagcagtactacacctacccctggaccttcggcggcggcaccaagctggagatcaag

SEQ ID NO: 35 - DNA, 3831 bp, synthetic construct
  misc_feature 1..3831: note = Optimized nucleotide sequence encoding Spike full-lenght
  source 1..3831: mol_type = other DNA
atgtttgtcttcctggtcctgctgcccctggtctcctctcagtgcgtgaacctgactactagaactcagctgcctcccgcttacaccaattccttcacccggggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaattgcacatttgagtacgtgtcccagcctttcctgatggacctggagggcaagcagggcaatttcaagaacctgagggagttcgtgtttaagaatatcgatggctacttcaaaatctactctaagcacacccccatcaacctggtgcgcgacctgcctcagggcttcagcgccctggagccactggtggatctgcctatcggcatcaacatcacccggtttcagacactgctggccctgcacagaagctacctgacacccggcgactcctctagcggatggaccgcaggagcagcagcctactatgtgggctatctgcagcctaggaccttcctgctgaagtacaacgagaatggcaccatcacagacgccgtggattgcgccctggatcctctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaaccaggttcgcaagcgtgtacgcatggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcacctggacagacaggcaagatcgccgactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactgcagtcctacggcttccagcccacaaacggcgtgggctatcagccttaccgcgtggtggtgctgagctttgagctgctgcacgcaccagcaacagtgtgcggacccaagaagtccaccaatctggtgaagaacaagtgcgtgaacttcaacttcaacggcctgaccggaacaggcgtgctgaccgagtccaacaagaagttcctgccatttcagcagttcggcagggacatcgcagataccacagacgccgtgcgcgacccacagaccctggagatcctggatatcacaccctgctctttcggcggcgtgagcgtgatcacaccaggaaccaatacaagcaaccaggtggccgtgctgtatcaggacgtgaattgtaccgaggtgcctgtggccatccacgccgatcagctgaccccaacatggcgggtgtacagcaccggctccaacgtgttccagacaagagcaggatgtctgatcggagcagagcacgtgaacaattcctatgagtgcgacatcccaatcggcgccggcatctgtgcctcttaccagacccagacaaactctccaaggagagcacggagcgtggcatcccagtctatcatcgcctataccatgtccctgggcgccgagaattctgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctgcagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagcacgggacctgatctgtgcccagaagtttaatggcctgaccgtgctgccacccctgctgacagatgagatgatcgcacagtacacaagcgccctgctggcaggaaccatcacatccggatggaccttcggcgcaggagccgccctgcagatcccctttgccatgcagatggcctataggttcaacggcatcggcgtgacccagaatgtgctgtacgagaaccagaagctgatcgccaatcagtttaactccgccatcggcaagatccaggacagcctgtcctctacagcctccgccctgggcaagctgcaggatgtggtgaatcagaacgcccaggccctgaataccctggtgaagcagctgagctccaacttcggcgccatctctagcgtgctgaatgatatcctgagccggctggacaaggtggaggcagaggtgcagatcgaccggctgatcacaggcagactgcagtctctgcagacctatgtgacacagcagctgatcagggcagcagagatcagggcaagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaataccttcgtgagcggcaactgtgacgtggtcatcggcatcgtgaacaataccgtgtatgatccactgcagcccgagctggacagctttaaggaggagctggataagtacttcaagaatcacacctcccctgacgtggatctgggcgacatcagcggcatcaatgcctccgtggtgaacatccagaaggagatcgaccgcctgaacgaggtggccaagaatctgaacgagagcctgatcgatctgcaggagctgggcaagtatgagcagtacatcaagtggccatggtacatctggctgggcttcatcgccggcctgatcgccatcgtgatggtgaccatcatgctgtgctgtatgacatcctgctgttcttgcctgaagggctgctgtagctgcggctcctgttgtaagtttgatgaagacgattccgagcctgtcctgaagggcgtgaagctgcactatacctctagataatga

SEQ ID NO: 36 - protein, 223 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 37 - protein, 20 aa, synthetic construct
  REGION 1..20: note = IgK signal peptide
METDTLLLWVLLLWVPGSTG

SEQ ID NO: 38 - protein, 228 aa, synthetic construct
  REGION 1..228: note = Fc domain
VDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 1275 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTSR

SEQ ID NO: 40 - protein, 471 aa, synthetic construct
  REGION 1..471: note = SARS-CoV-2 RBD fused to IgK signal peptide and to hFc
METDTLLLWVLLLWVPGSTGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 1413 bp, synthetic construct
  misc_feature 1..1413: note = Nucleotide sequence encoding SARS-CoV-2 RBD fused to IgK signal peptide and to hFc
  source 1..1413: mol_type = other DNA
atggagacagacacactcctgctatgggtactgctgctctgggttccaggatccacaggaagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcgtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 42 - protein, 8 aa, synthetic construct
  REGION 1..8: note = CDR region
GYTFTEYT

SEQ ID NO: 43 - protein, 8 aa, synthetic construct
  REGION 1..8: note = CDR region
INPNNGDS

SEQ ID NO: 44 - protein, 13 aa, synthetic construct
  REGION 1..13: note = CDR region
ARDGYPNSYAMDY

SEQ ID NO: 45 - protein, 12 aa, synthetic construct
  REGION 1..12: note = CDR region
QSLLYSTNQKNY

SEQ ID NO: 46 - protein, 9 aa, synthetic construct
  REGION 1..9: note = CDR region
QQYYSYPWT

SEQ ID NO: 47 - protein, 8 aa, synthetic construct
  REGION 1..8: note = CDR region
INPYIGDT

SEQ ID NO: 48 - protein, 13 aa, synthetic construct
  REGION 1..13: note = CDR region
ARDGYPDYSAMDF

SEQ ID NO: 49 - protein, 12 aa, synthetic construct
  REGION 1..12: note = CDR region
QNLLYSSNNKNY

SEQ ID NO: 50 - protein, 9 aa, synthetic construct
  REGION 1..9: note = CDR region
QQYYTYPWT

SEQ ID NO: 51 - protein, 122 aa, synthetic construct
  REGION 1..122: note = Heavy chain variable region (VH) of antibody 3-12B12-F4
EVQLQQSGAELVRPGALVKLSCKASGFNIKDYYMHWVKQRPEQGLEWIGWIDPENGNSIYDPKFQGKASITADTSFNTVNLHLSSLTSEDTAVYYCAPYYYDSTYVGTMDYWGQGTSVTVSS

SEQ ID NO: 52 - protein, 107 aa, synthetic construct
  REGION 1..107: note = Light chain variable region (VL) of antibody 3-12B12-F4
QIVLTQSPAIMSASPGEKVTMTCSASSSVGYMYWYQQKPGSSPRLLIYDTSNLASGVPVRFGGSGSGTSYSLTISRMEAEDAATYYCQQWSSYPPRTFGGGTKLEIK

SEQ ID NO: 53 - DNA, 366 bp, synthetic construct
  misc_feature 1..366: note = Nucleotide sequence encoding heavy chain variable region (VH) of antibody 3-12B12-F4
  source 1..366: mol_type = other DNA
gaggtgcagctgcagcagagcggcgccgagctggtgaggcccggcgccctggtgaagctgagctgcaaggccagcggcttcaacatcaaggactactacatgcactgggtgaagcagaggcccgagcagggcctggagtggatcggctggatcgaccccgagaacggcaacagcatctacgaccccaagttccagggcaaggccagcatcaccgccgacaccagcttcaacaccgtgaacctgcacctgagcagcctgaccagcgaggacaccgccgtgtactactgcgccccctactactacgacagcacctacgtgggcaccatggactactggggccagggcaccagcgtgaccgtgagcagc

SEQ ID NO: 54 - DNA, 321 bp, synthetic construct
  misc_feature 1..321: note = Nucleotide sequence encoding light chain variable region (VL) of antibody 3-12B12-F4
  source 1..321: mol_type = other DNA
cagatcgtgctgacccagagccccgccatcatgagcgccagccccggcgagaaggtgaccatgacctgcagcgccagcagcagcgtgggctacatgtactggtaccagcagaagcccggcagcagccccaggctgctgatctacgacaccagcaacctggccagcggcgtgcccgtgaggttcggcggcagcggcagcggcaccagctacagcctgaccatcagcaggatggaggccgaggacgccgccacctactactgccagcagtggagcagctacccccccaggaccttcggcggcggcaccaagctggagatcaag